(19) United States
(12) Patent Application Publication
(10) Pub. No.: US 2024/0132548 A1
(43) Pub. Date: Apr. 25, 2024

(54) STABILIZED PRE-FUSION RSV FB ANTIGENS

(71) Applicant: Janssen Vaccines & Prevention B.V., Leiden (NL)

(72) Inventors: Johannes Petrus Maria LANGEDIJK, Amsterdam (NL); Tina RITSCHEL, Oestgeest (NL); Mark Johannes Gerardus BAKKERS, Haarsteeg (NL)

(21) Appl. No.: 18/546,798

(22) PCT Filed: Feb. 18, 2022
(86) PCT No.: PCT/EP2022/054128

§ 371 Date: Aug. 16, 2023

Related U.S. Application Data
(60) Provisional application No. 63/151,262, filed on Feb. 19, 2021.

(30) Foreign Application Priority Data
Mar. 28, 2021 (EP) 21165577.4

(51) Int. Cl. C07K 14/005; A61P 37/04; C12N 15/86

(52) U.S. Cl. CPC C07K 14/005; A61P 37/04; C12N 15/86; C12N 2710/10043

(57) ABSTRACT

The present invention provides stable pre-fusion respiratory syncytial virus (RSV) FB proteins, nucleic acid molecules and vectors encoding such proteins, and compositions comprising said proteins, nucleic acid molecules and/or vectors, and uses thereof for the prevention and/or treatment of RSV infection.

The present invention relates to the field of medicine. The invention in particular relates to recombinant pre-fusion RSV F_{B} proteins and fragments thereof and to nucleic acid molecules encoding the RSV FB proteins and fragments thereof, and to uses thereof, e.g. in vaccines.

### BACKGROUND OF THE INVENTION

After discovery of the respiratory syncytial virus (RSV) in the 1950s, the virus soon became a recognized pathogen associated with lower and upper respiratory tract infections in humans. Worldwide, it is estimated that 64 million RSV infections occur each year resulting in 160.000 deaths (WHO Acute Respiratory Infections Update September 2009). The most severe disease occurs particularly in premature infants, the elderly and immunocompromised individuals. In children younger than 2 years, RSV is the most common respiratory tract pathogen, accounting for approximately 50% of the hospitalizations due to respiratory infections, with the peak of hospitalization occurring at 2-4 months of age. It has been reported that almost all children have been infected by RSV by the age of two. Repeated infection during lifetime is attributed to ineffective natural immunity. In the elderly, the RSV disease burden is similar to that caused by non-pandemic influenza A infections.

RSV is a paramyxovirus, belonging to the subfamily of Pneumoviridae. Its genome encodes for various proteins, including the membrane proteins known as RSV Glycoprotein (G) and RSV fusion (F) protein which are the major antigenic targets for neutralizing antibodies. Antibodies against the F protein can prevent virus entry into the cell and thus have a neutralizing effect.

RSV F fuses the viral and host-cell membranes by irreversible protein refolding from the labile pre-fusion conformation to the stable post-fusion conformation. Structures of both conformations have been determined for RSV F (McLellan J S, et al. (2010, 2013, 2013); Swanson K A, et al. (2011)), as well as for the fusion proteins from related paramyxoviruses, providing insight into the complex mechanism this fusion protein undergoes. Like other class I fusion proteins, the inactive precursor, RSV F₀, requires cleavage during intracellular maturation by a furin-like protease. RSV F contains two furin cleavage sites, which leads to three proteins: F2, p27 and F1. The p27 fragment is not part of the mature F protein and F2 and F1 are associated by two disulfide bridges, with the latter containing a hydrophobic fusion peptide (FP) at its N-terminus. In order to refold from the pre-fusion to the post-fusion conformation, the refolding region 1 (RR1) between residue 137 and 216, that includes the FP and heptad repeat A (HRA) has to transform from an assembly of helices, loops and strands to a long continuous helix. The FP, located at the N-terminal segment of RR1, is then able to extend away from the viral membrane and to insert into the proximal membrane of the target cell. Next, the refolding region 2 (RR2), which forms the C-terminal stem in the pre-fusion F spike and includes the heptad repeat B (HRB), relocates to the other side of the RSV F head and binds the HRA coiled-coil trimer with the HRB domain to form the six-helix bundle. The formation of the RR1 coiled-coil and relocation of RR2 to complete the six-helix bundle are the most dramatic structural changes that occur during the refolding process.

A vaccine preventing against RSV infection is currently not yet available, but it is highly desired due to the high disease burden. The RSV fusion glycoprotein (RSV F) is an attractive vaccine antigen as it is the principal target of neutralizing antibodies in human sera.

Most neutralizing antibodies in human sera are directed against the pre-fusion conformation, but due to its instability the pre-fusion conformation has a propensity to prematurely refold into the post-fusion conformation, both in solution and on the surface of the virions. As indicated above, crystal structures have revealed a large conformational change between the pre-fusion and post-fusion states. The magnitude of the rearrangement suggested that only a portion of antibodies directed to the post-fusion conformation of RSV-F will be able to cross react with the native conformation of the pre-fusion spike on the surface of the virus. Accordingly, efforts to produce a vaccine against RSV have focused on developing vaccines that contain pre-fusion forms of RSV F protein (see, e.g., WO020101149745, WO2010/1149743, WO2009/1079796, WO2012/158613). These efforts so far have been focused on RSV F proteins derived from RSV A strains, and until this date, still no vaccine is available.

Human RSV (HRSV) is divided into two main subtypes; HRSV A and HRSV B, that are generally distinguished based on sequence differences in the G protein. Although the F proteins of A (F_{A}) and B (F_{B}) strains show a high degree of sequence identity (˜95% in the mature ectodomain), it is not known if the cross reactivity of anti-F antibodies is broad enough.

A need remains for efficacious vaccines against RSV. The present invention aims at providing means for obtaining stabilized pre-fusion RSV F_{B} proteins for use in vaccines against RSV.

### SUMMARY OF THE INVENTION

The present invention provides recombinant stabilized pre-fusion RSV fusion (F) proteins, comprising an F1 and an F2 domain comprising an amino acid sequence of the F1 and F2 domain of an F protein of an RSV B strain (RSV FB proteins), and fragments thereof.

The invention also provides nucleic acid molecules encoding the pre-fusion RSV F_{B} proteins, or fragments thereof, as well as vectors, e.g. adenovectors, comprising such nucleic acid molecules.

The invention further provides compositions, preferably immunogenic compositions or vaccines, comprising an RSV FB protein, a nucleic acid molecule and/or a vector, as described herein, and the use thereof in inducing an immune response against RSV F protein, in particular the use thereof as a vaccine against RSV.

The invention also provides methods for inducing an anti-respiratory syncytial virus (RSV) immune response in a subject, comprising administering to the subject an effective amount of a pre-fusion RSV FB protein, a nucleic acid molecule encoding said RSV F_{B} protein, and/or a vector comprising said nucleic acid molecule, as described herein. Preferably, the induced immune response is characterized by the induction of neutralizing antibodies and/or a cellular response against RSV and/or protective immunity against RSV infection.

The invention in particular provides methods for vaccinating a subject against RSV, the methods comprising administering to the subject a composition or vaccine as described herein.

The invention furthermore provides methods for preventing infection and/or replication of RSV in a subject, the methods comprising administering to the subject a composition or vaccine as described herein.

### BRIEF DESCRIPTION OF THE FIGS.

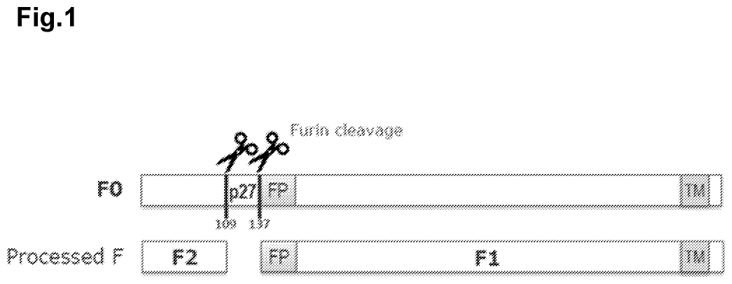

FIG. 1: Schematical representation of RSV F protein. F0 is enzymatically processed into F1 and F2 subunits by a furin-like protease at two positions which results in release of the p27 peptide in the mature processed protein. F1 and F2 are joined together by disulfide bonds (not shown).

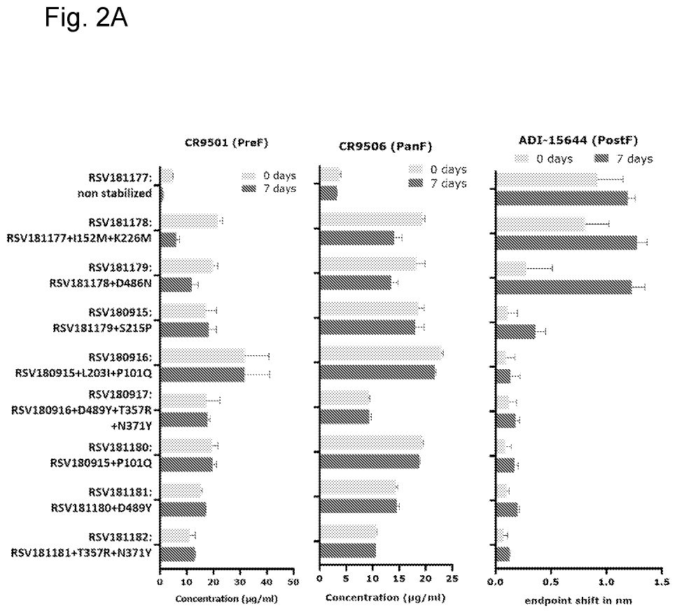

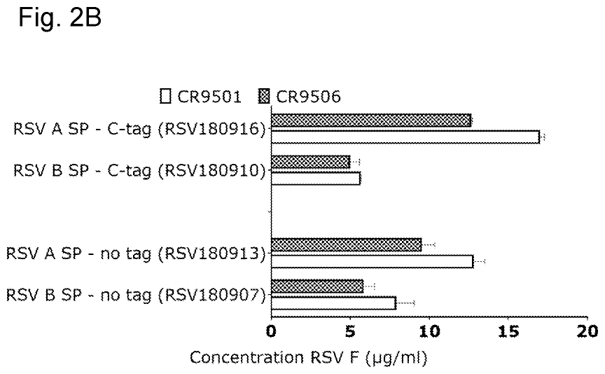

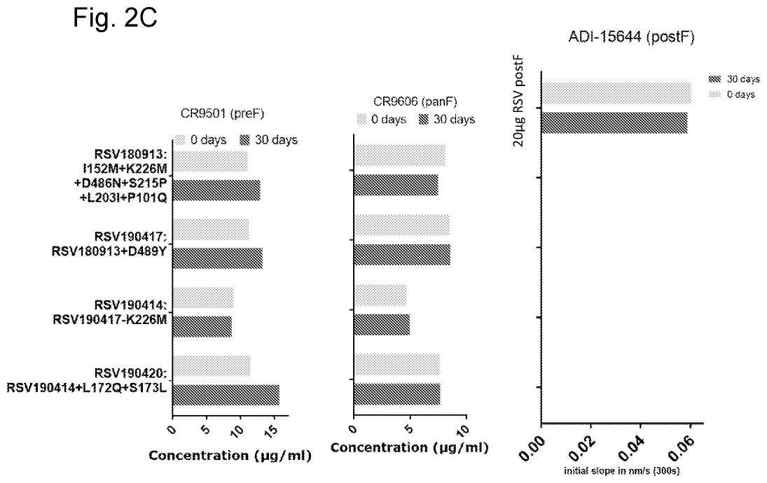

FIG. 2: Analysis of cell culture supernatant after transfection measured with BioLayer Interferometry. RSV F concentrations and stability of non-stabilized and stabilized F variants as described in Example 2 in supernatant are shown. The total polypeptide content and the pre-fusion content were measured by CR9506 and CR9501 binding, respectively. The post-fusion content of polypeptide was measured by ADI-15644 (Gilman et al., 2016) binding. The non-stabilized F protein RSV181177 (SEQ ID NO: 3) was compared to stabilized variants at the day of harvest and 7 days later (A). RSV F expression levels of RSV F_{B} variants with either an RSV type A (RSV180816 and RSV180913) or RSV type B (RSV180910 and RSV180907) signal peptide, with C-tag or without tag on the day of harvest (B). Pre-fusion F expression levels of tag-free RSV F_{B} variants with several stabilizing amino acid substitutions: RSV180913 (I152M+K226M+D486N+S215P+L203I+P101Q, SEQ ID NO: 14) and additional stabilizing mutation D489Y (RSV190417; SEQ ID NO: 15), a wild type amino acid residue at position 226 (K226) (RSV190414, SEQ ID NO: 16) and drift mutations L172Q+S173L (RSV190420; SEQ ID NO: 17) at day 0 and day 30 after harvest (for the post F evaluation, a positive control of 20 μg RSV post F protein spiked into supernatant of mock-transfected cells was taken along) (C). FIG. 2A and B show the average and error bars of two independent transfections. Data in FIG. 2C is based on one transfection.

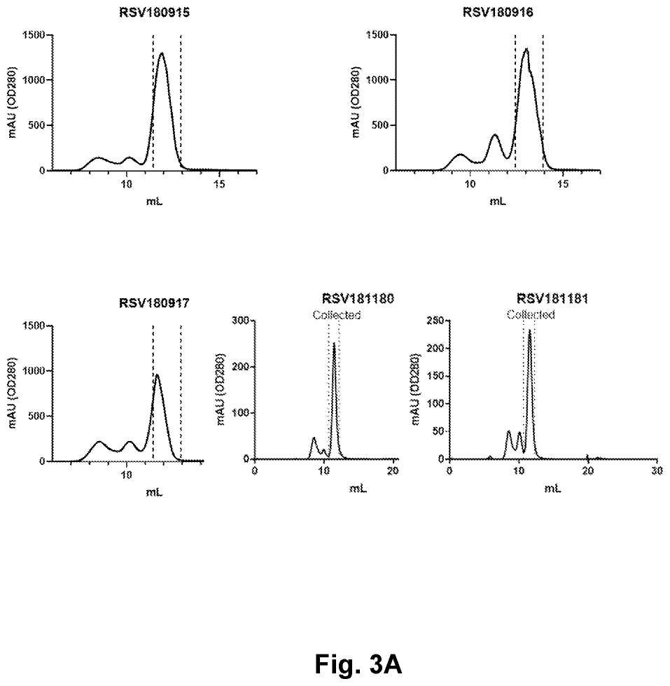

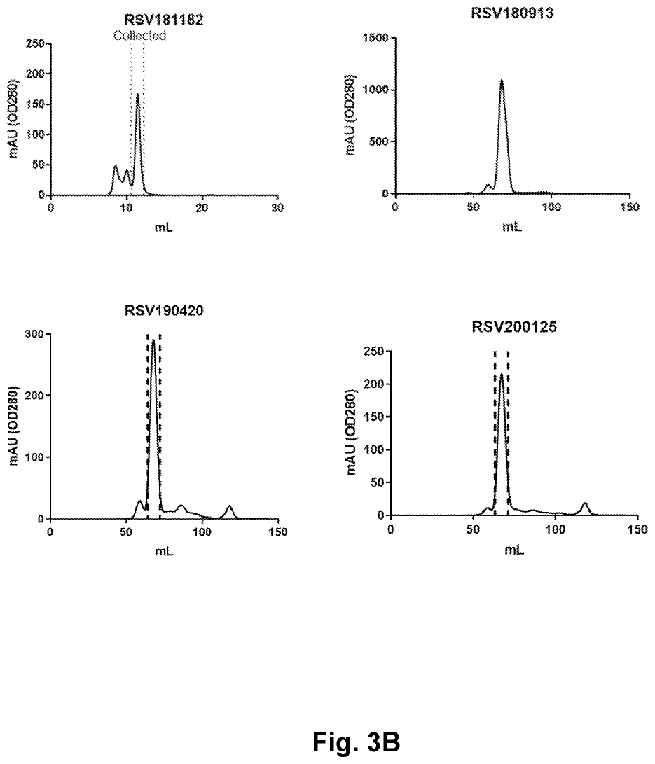

FIG. 3: SEC profiles of the last purification step of selected protein variants as described in Example 3. Protein fraction was collected between the 2 vertical dashed lines.

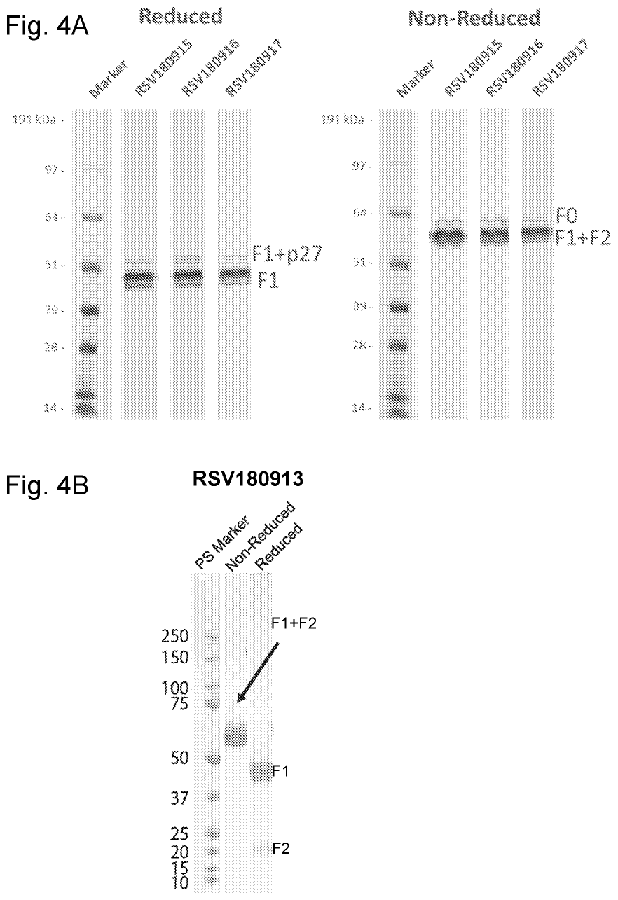

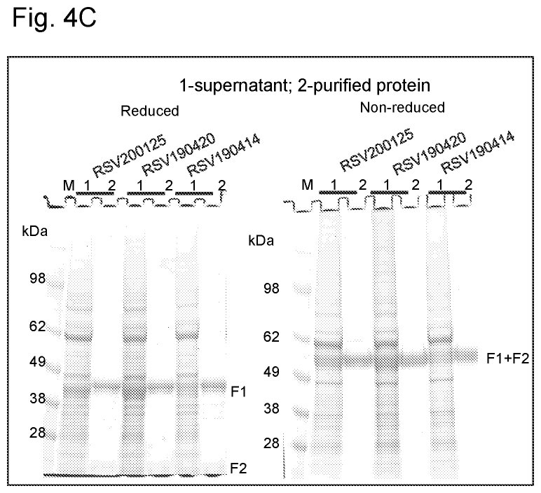

FIG. 4: SDS-PAGE analysis. Western blot of pooled fractions of RSV180915 (SEQ ID NO: 6), RSV180916 (SEQ ID NO: 8) and RSV180917 (SEQ ID NO: 9) under non-reducing and reducing conditions (A). In B and C, the gels are Coomassie stained. RSV190913 (SEQ ID NO: 14) protein sample containing pooled peak from the SEC chromatogram under non-reducing and reducing conditions (B). RSV190414 (SEQ ID NO: 16), RSV190420 (SEQ ID NO: 17) and RSV200125 (SEQ ID NO: 18) SDS-PAGE of crude harvest (1) and purified F protein (2) under non-reducing (right panel) and reducing (left panel) conditions (C).

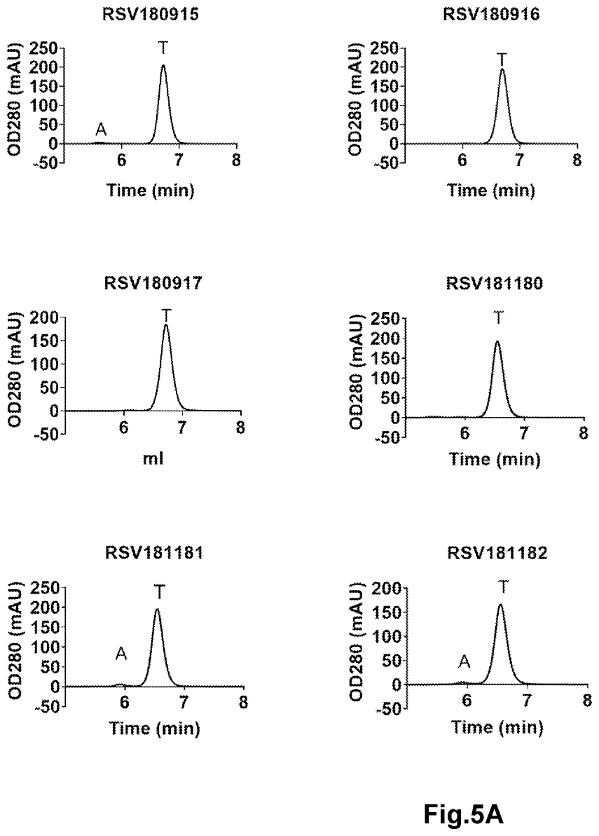

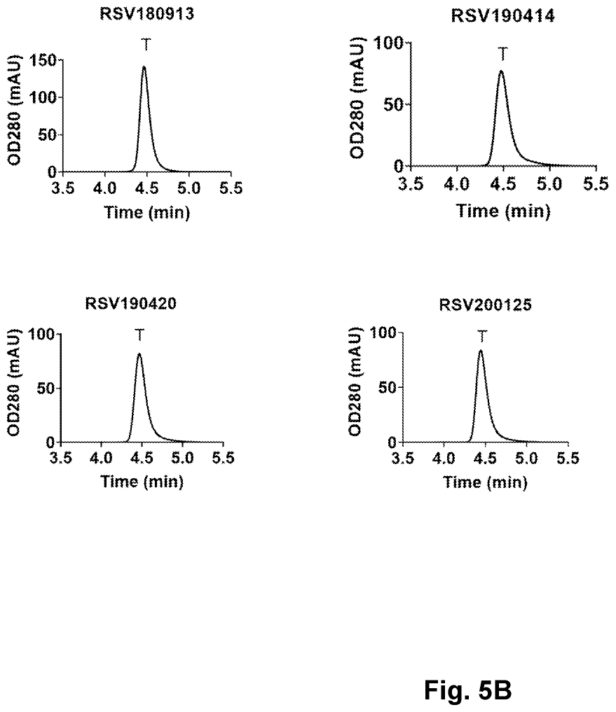

FIG. 5: Analytical SEC analysis of the purified F proteins. Aggregates and trimers are indicated with A and T, respectively. The proteins have been evaluated with HPLC or UPLC with a trimer retention time of about 6.5 minutes or 4.5 minutes, respectively.

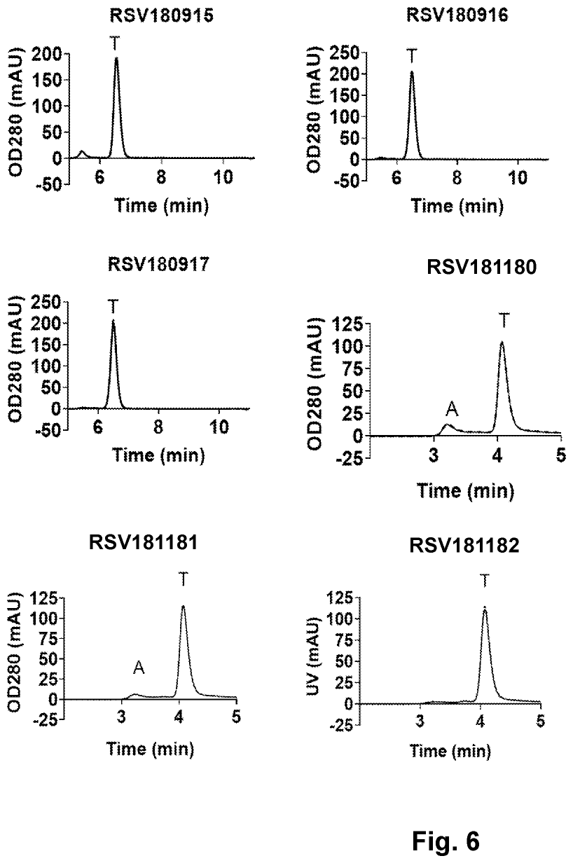

FIG. 6: Analytical SEC analysis of the purified RSV preF type B proteins (n=2) after storage for 37° C. for 35 days. Aggregates and trimers are indicated with A and T, respectively. The proteins have been evaluated on with HPLC or UPLC with a trimer retention time of about 6.5 minutes or 4.5 minutes, respectively.

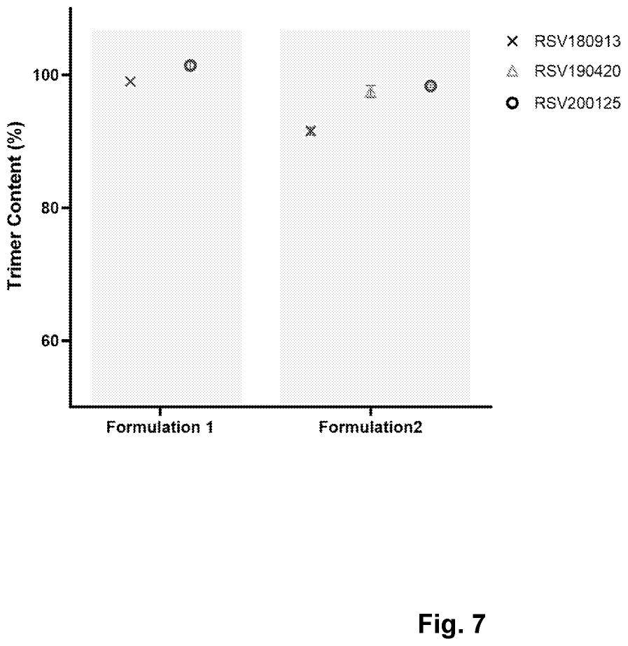

FIG. 7: Cryo stability of purified RSV preF type B polypeptides of RSV180913 (SEQ ID NO: 14), RSV19420 (SEQ ID NO: 17) and RSV200125 (SEQ ID NO: 18). Residual pre-fusion trimer percentage as measured by analytical SEC after a slow freeze process in different formulation buffers. Trimer content for control sample kept at 4° C. was set at 100%. Averaged data±SD.

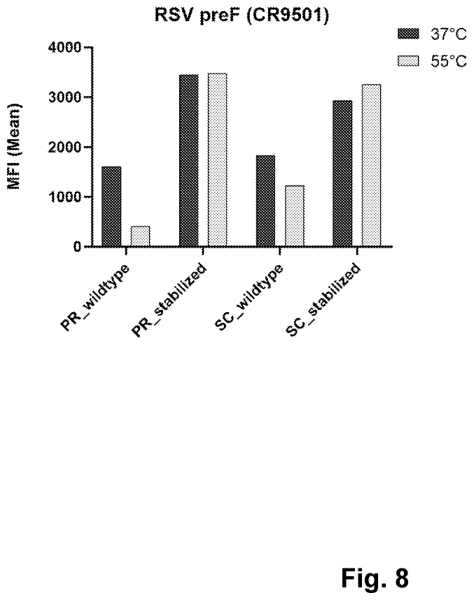

FIG. 8: Full-length RSV-B F proteins in FACS. Transient expression of polypeptides in expiHEK293F cells for 2 days followed by 10 min heat stress at 37° C. or 55° C. Surface expression of PreF protein was measured with monoclonal antibody CR9501 which is specific for the pre-fusion conformation of RSV F.

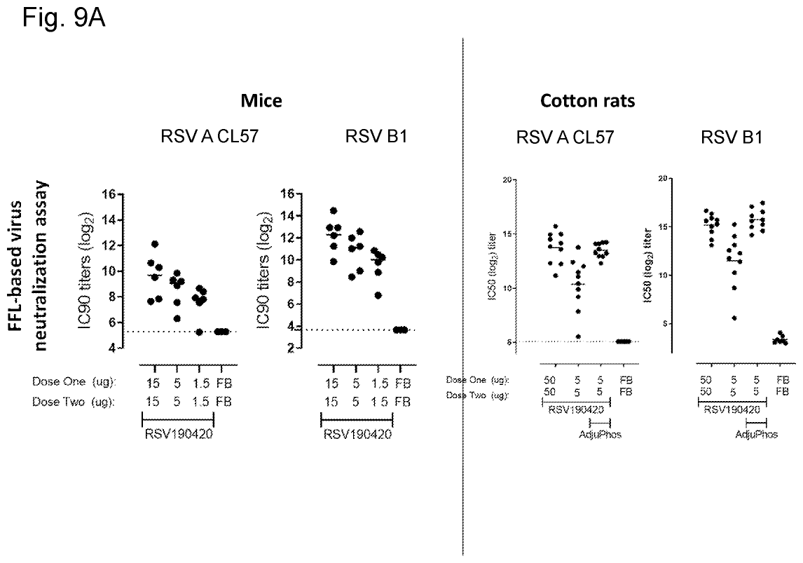

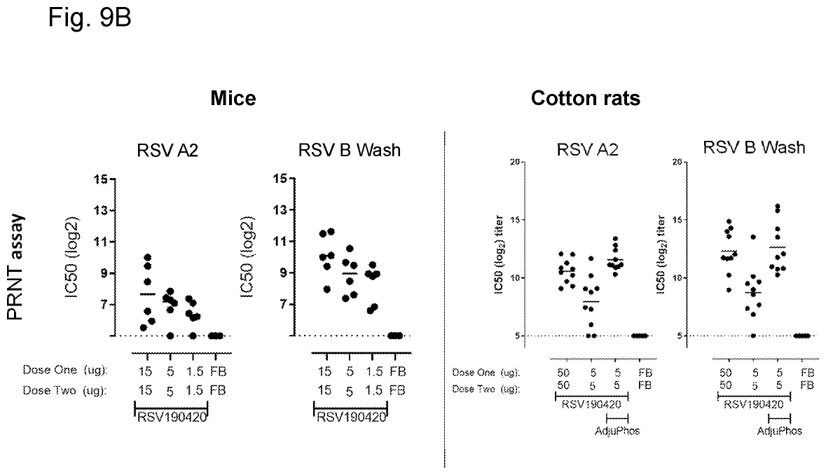

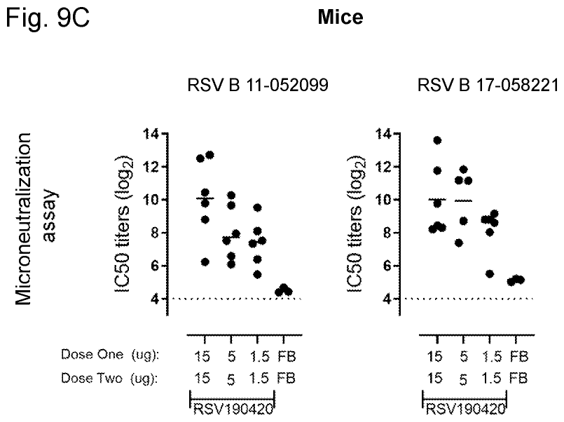

FIG. 9: Immunogenicity of preFB RSV190420 (SEQ ID NO: 17) in mice and cotton rats. RSV preF_{B} protein was administrated as intramuscular immunization in mice and cotton rats at day 0 and day 28. Virus neutralizing antibody titers against the RSV strains indicated were determined by firefly luciferase-based assay (A), Plaque Reduction Neutralization Test (B), or microneutralization assay (C) 2 weeks (mice) or 3 weeks (cotton rats) after the final immunization. Symbols represent neutralizing titers of individual animals, whereas mean titers are indicated with horizontal lines. Lower limit of detection or qualification is indicated with a dotted line. FB: formulation buffer.

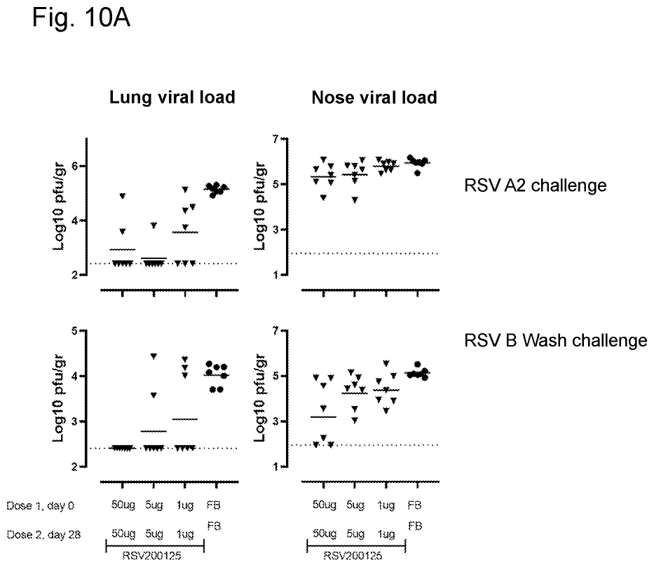

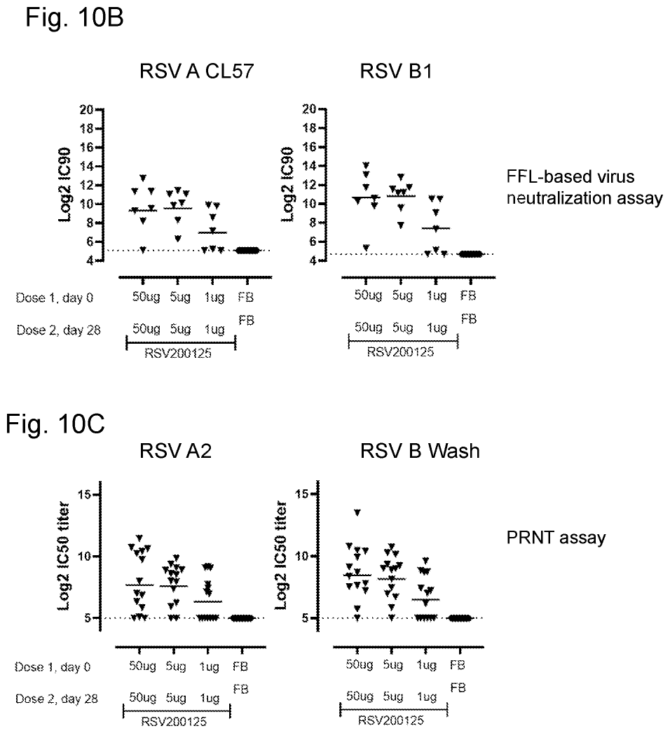

FIG. 10: Immunogenicity and protective efficacy of preF-B RSV200125 (SEQ ID NO: 18) in cotton rats. RSV preF-B protein was administrated as intramuscular immunization in cotton rats at day 0 and day 28, and animals were intranasally challenged at day 49 with RSV A2, or at day 50 with RSV B Wash. Lung and nose viral load was determined by plaque assay in tissue homogenates isolated 5 days post challenge (A). Pre-challenge serum samples were analyzed for neutralizing antibodies against the RSV strains indicated by a firefly luciferase-based assay (B), or Plaque Reduction Neutralization Test (C). Symbols represent viral load or neutralizing titers of individual animals, whereas mean titers are indicated with horizontal lines. Lower limit of detection or qualification is indicated with a dotted line. FB: formulation buffer.

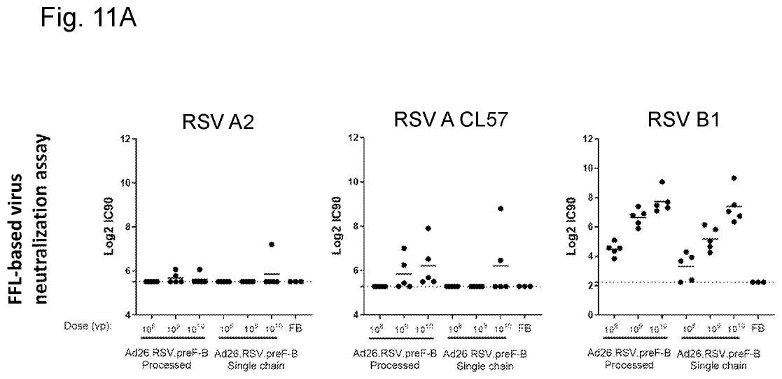

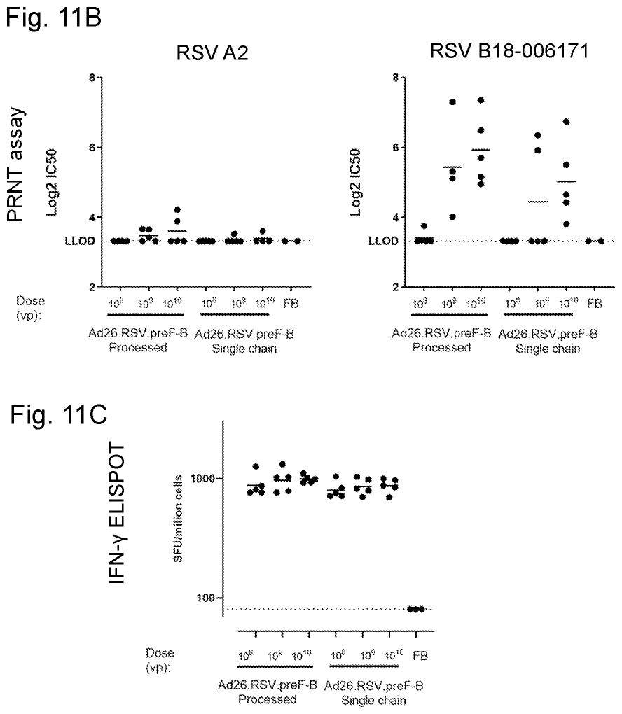

FIG. 11: Immunogenicity of Ad26 encoding processed or single chain variants of preF-B (SEQ ID NO: 32 and 34) in mice. Mice were immunized with different dose levels of Ad26.RSV.preF-B processed or single chain. At 6 weeks post immunization, virus neutralizing antibody titers against the RSV strains indicated were determined by firefly luciferase-based assay (A), or Plaque Reduction Neutralization Test (B). RSV F directed cellular immune responses were determined in splenocytes isolated at 6 weeks post immunization by IFN-γ ELISPOT assay. Symbols represent responses of individual animals, whereas mean responses are indicated with horizontal lines. Lower limit of detection or qualification is indicated with a dotted line. FB: formulation buffer.

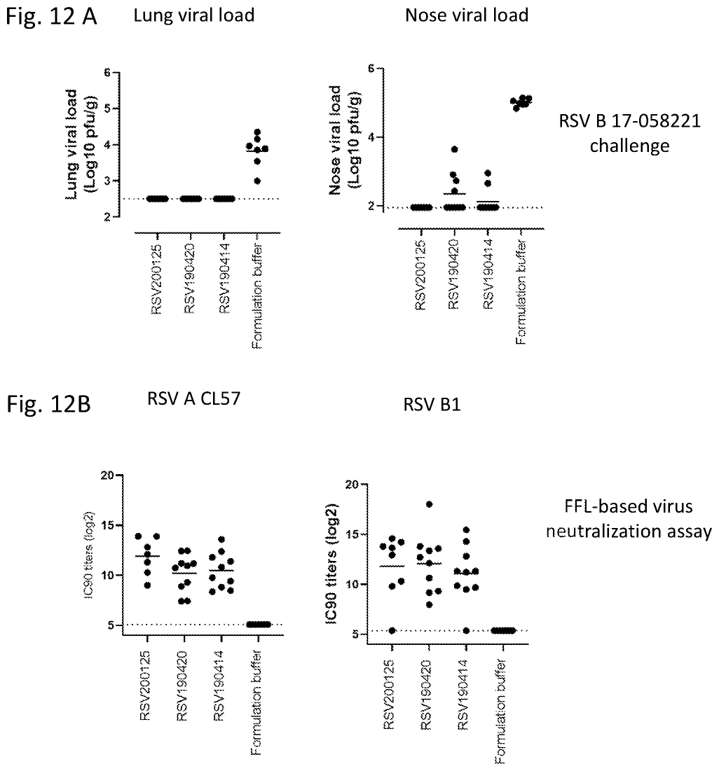

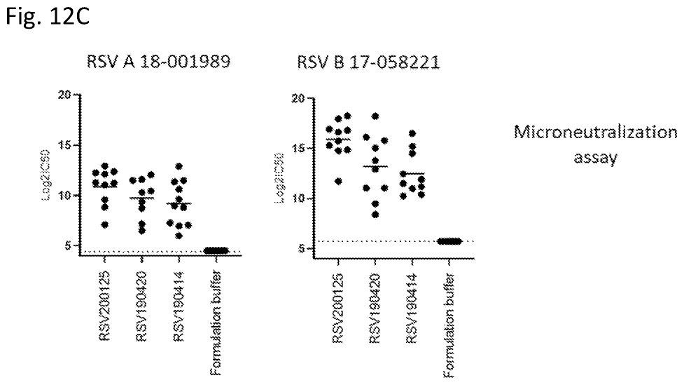

FIG. 12: Immunogenicity and protective efficacy of preF-B proteins RSV190414 (SEQ ID NO: 16), RSV190420 (SEQ ID NO: 17) and RSV200125 (SEQ ID NO: 18) in cotton rats. RSV preF-B proteins (50 μg) were administrated as intramuscular immunization in cotton rats at day 0 and day 28, and animals were intranasally challenged at day 49 with RSV B17-058221. Lung and nose viral load was determined by plaque assay in tissue homogenates isolated 5 days post challenge (A). Pre-challenge serum samples were analyzed for neutralizing antibodies against the RSV strains indicated by a firefly luciferase-based assay (B), or by microneutralization assay (C). Symbols represent viral load or neutralizing titers of individual animals, whereas mean titers are indicated with horizontal lines. Lower limit of detection or qualification is indicated with a dotted line.

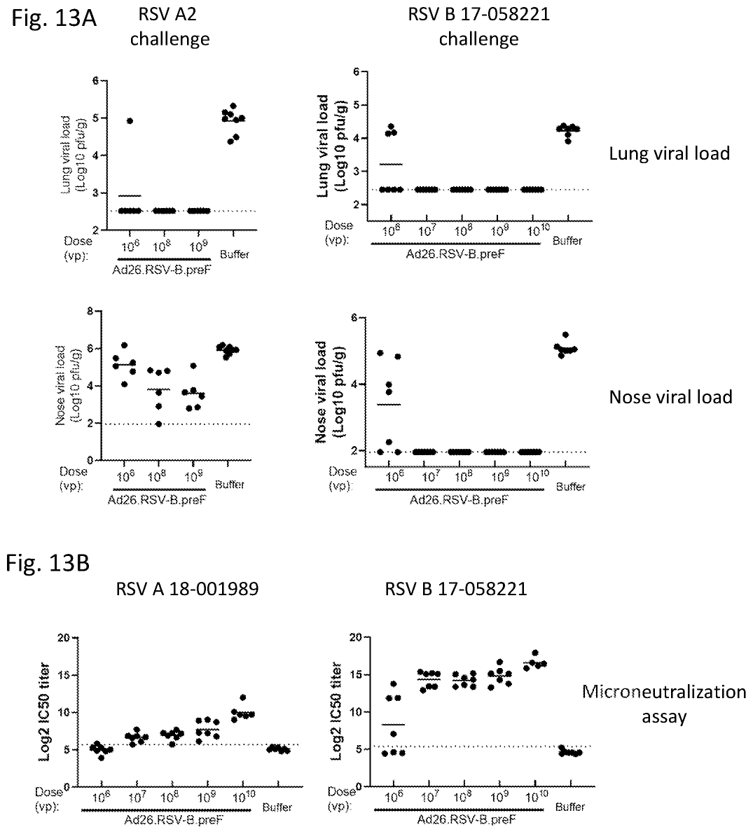

FIG. 13: Immunogenicity and protective efficacy of Ad26 encoding processed preF-B (SEQ ID NO: 32 in cotton rats. Ad26.RSV-B.preF was administrated as intramuscular immunization in cotton rats at day 0, and animals were intranasally challenged at day 49 with RSV A2 or RSV B 17-058221. Lung and nose viral load was determined by plaque assay in tissue homogenates isolated 5 days post challenge (A). Pre-challenge serum samples were analyzed for neutralizing antibodies against the RSV strains indicated by microneutralization assay (B). Symbols represent viral load or neutralizing titers of individual animals, whereas mean titers are indicated with horizontal lines. Lower limit of detection or qualification is indicated with a dotted line.

### DETAILED DESCRIPTION OF THE INVENTION

Human RSV (HRSV) is a common contributor of respiratory infections causing bronchitis, pneumonia, and chronic obstructive pulmonary infections in people of all ages. The fusion protein (F protein) of the respiratory syncytial virus (RSV) is involved in fusion of the viral membrane with a host cell membrane, which is required for infection. RSV F mRNA is translated into a 574 amino acid precursor protein designated F0, which contains a signal peptide sequence at the N-terminus (e.g. amino acid residues 1-25 of SEQ ID NO: 1) which is removed by a signal peptidase in the endoplasmic reticulum. F0 is cleaved at two furin cleavage sites (between amino acid residues 109/110 and 136/137) by cellular proteases (in particular furin, or furin-like proteases) removing a short glycosylated intervening sequence (also referred to a p27 region, comprising the amino acid residues 110 to 136, and generating two domains (or subunits) designated F1 and F2 (FIG. 1).

The F1 domain (amino acid residues 137-574) contains a hydrophobic fusion peptide at its N-terminus and the C-terminus contains the transmembrane (TM) (amino acid residues 530-550) and cytoplasmic region (amino acid residues 551-574). The F2 domain (amino acid residues 26-109) is covalently linked to F1 by two disulfide bridges. The F1-F2 heterodimers are assembled as homotrimers in the virion. According to the present invention a “processed RSV F protein” refers to the RSV F protein after cleavage at the furin cleavage sites, i.e. without signal peptide and the p27 region.

As described above, a vaccine against RSV infection is currently not yet available. One potential approach to producing a vaccine is providing a subunit vaccine based on purified RSV F protein. However, for this approach it is desirable that the purified RSV F protein is in a conformation which resembles the conformation of the pre-fusion state of RSV F protein and is stable over time. Efforts thus have been focused on RSV F proteins that have been stabilized in the pre-fusion conformation.

Human RSV is divided into two major antigenic groups of strains, subtypes A and B, that are largely defined by genetic variation in the G glycoprotein. These subtypes show an irregular, alternating prevalence pattern, with subtype A having a higher cumulative prevalence than subtype B. The F protein is highly conserved between RSV A and B and induces neutralizing antibodies across the two groups. However, although the F proteins of A and B strains show a high degree of sequence identity (˜95% in the mature ectodomain), it is not known if the cross reactivity of anti-F antibodies is broad enough and if a vaccine based on RSV FA protein could protect against infection by RSV B strains.

The present invention provides novel stabilized recombinant pre-fusion RSV fusion (F) proteins, comprising an F1 and an F2 domain comprising an amino acid sequence of the F1 and F2 domain of an F protein of an RSV B strain, wherein the amino acid residue at position 101 is Q, the amino acid residue at position 152 is M, the amino acid residue at position 215 is P, and the amino acid residue at position 486 is N. The invention thus provides stabilized recombinant pre-fusion F proteins of RSV subgroup B (RSV F_{B}) proteins, or fragments thereof. The numbering of the numbering of the positions of the amino acid residues is according to the numbering of the amino acid residues in SEQ ID NO: 1. According to the invention it has been demonstrated that the presence of the specific amino acids at the indicated positions increases the stability of the proteins in the pre-fusion conformation. According to the invention, the specific amino acids may be already present in the amino acid sequence of the RSV FB protein, or may be introduced by substitution (mutation) of a naturally occurring amino acid residue at that position into the specific amino acid residue according to the invention. According to the invention, the proteins thus may comprise one or more mutations in their amino acid sequence as compared to the amino acid sequence of a wild type RSV F_{B} protein.

According to the invention the term “stabilized pre-fusion protein” refers to a protein which is stabilized in the pre-fusion conformation, i.e. that comprises at least one epitope that is specific to the pre-fusion conformation of the RSV F protein, e.g. as determined by specific binding of an antibody that is specific for the pre-fusion conformation to the proteins, and can be produced (expressed) in sufficient quantities.

In certain embodiments, the amino acid residue at position 203 is I. According to the invention it was shown that the presence of I at position 203 further improves stability of the RSV F_{B} protein, in particular in soluble RSV F_{B} proteins.

Alternatively, or in addition, the amino acid residue at position 489 is Y. According to the invention it was shown that the polypeptide stability is improved by the presence of this amino acid residue at the indicated position.

In certain embodiments, the amino acid residue at position 226 is M. The amino acid M at position 226 increases the stability and expression of the protein.

In a preferred embodiment, the amino acid residue at position 101 is Q, the amino acid residue at position 152 is M, the amino acid residue at position 203 is I, the amino acid residue at position 215 is P, and the amino acid residue at position 486 is N, and the amino acid at position 357 is not R, and/or the amino acid residue at position 371 is not Y.

In a preferred embodiment, the amino acid residue at position 101 is Q, the amino acid residue at position 152 is M, the amino acid residue at position 203 is I, the amino acid residue at position 215 is P, the amino acid residue at position 486 is N, and the amino acid residue at position 489 is Y.

In a preferred embodiment, the amino acid residue at position 101 is Q, the amino acid residue at position 152 is M, the amino acid residue at position 203 is I, the amino acid residue at position 215 is P, the amino acid residue at position 486 is N, and the amino acid residue at position 489 is Y, and the amino acid at position 357 is not R, and/or the amino acid residue at position 371 is not Y.

Chen et al. (Sci Rep. 8(1): 4491, 2018) have reported emerging drift mutations L172Q and S173L in 2015-2016 circulating virus populations. In addition, Lu et al. (Sci Rep. 9(1): 3898, 2019) have described that L172Q & S173L are fixed and that K191R, 1206M & Q209R have arisen for strains of 2015-2018. Based on sequences of recently circulating strains (2018-2019) deposited in ViPR and GISAID it appears that all five positions are fixed now. Thus, in certain embodiments, the amino acid residue at position 172 is Q and the amino acid residue at position 173 is L. Alternatively, or in addition, the amino acid residue at position 191 is R, the amino acid residue at position 206 is M and the amino acid residue at position 209 is R. Thus, the RSV FB proteins more closely resemble the RSV F protein of circulating RSV B strains.

In a preferred embodiment, the amino acid residue at position 101 is Q, the amino acid residue at position 152 is M, the amino acid residue at position 172 is Q and the amino acid residue at position 173 is L, the amino acid residue at position 215 is P, the amino acid residue at position 486 is N, and the amino acid residue at position 489 is Y, and optionally the amino acid residue at position 203 is I.

In another preferred embodiment, the amino acid residue at position 101 is Q, the amino acid residue at position 152 is M, the amino acid residue at position 172 is Q and the amino acid residue at position 173 is L, the amino acid residue at position 191 is R, the amino acid residue at position 206 is M and the amino acid residue at position 209 is R, the amino acid residue at position 215 is P, the amino acid residue at position 486 is N, and optionally the amino acid residue at position 203 is I and/or the amino acid residue at position 489 is Y.

In certain embodiments, the present invention provides recombinant pre-fusion F proteins as described herein wherein the amino acid residue at position 357 is not R and the amino acid residue at position 372 is not Y.

The RSV F_{B} proteins according to the invention may comprise the naturally occurring furin cleavage sites. In certain embodiments, the furin cleavage sites may have been deleted.

Deletion of the furin cleavage site may comprise deletion of the p27 peptide. In these embodiments, the F protein will remain a “single chain” protein, i.e. will not be processed by furin into F1 and F2. In certain embodiments, the furin cleavage site has been deleted by deletion of the p27 peptide, comprising deletion of the amino acids 109-135, and replacement of the deleted p27 peptide by a linker (or linking sequence, e.g. GSGSG) linking the F1 and F2 domains, optionally in combination with a mutation of the amino acid R at position 106 into Q (R106Q) and the amino acid F at position 137 into S (F137S).

In certain embodiments, the proteins comprise a truncated F1 domain. Thus, in order to obtain a soluble RSV F_{B} protein, the transmembrane (TM) and the cytoplasmic region may be deleted to create a soluble secreted F protein (sF protein). As used herein a “truncated” F1 domain refers to a F1 domain that is not a full length F1 domain, i.e. wherein either N-terminally or C-terminally one or more amino acid residues have been deleted. According to the invention, at least the transmembrane domain and cytoplasmic tail have been deleted to permit expression as a soluble ectodomain. In certain embodiments the F1 domain has been truncated after the amino acid at position 513 i.e. the amino acids from 514 to 574 have been deleted.

In certain embodiments, a heterologous trimerization domain has been linked to the C-terminus of the truncated F1 domain, either directly or by using a linker (e.g. a linking sequence SAIG). Because the TM region is responsible for membrane anchoring and increases stability, the anchorless soluble F protein is considerably more labile than the full-length protein and will even more readily refold into the post-fusion end-state. Thus, in order to obtain stabilized soluble FB proteins in the pre-fusion conformation that show high expression levels a heterologous trimerization domain may be fused to the C-terminal end of the truncated F1 domain of the RSV F_{B} protein either directly or by using a linker (e.g. a linking sequence SAIG). For example, for the trimerization of a soluble RSV F protein, a fibritin—based trimerization domain may be fused to the C-terminus of the ectodomain (McLellan et al., (2010, 2013)). This fibritin domain or ‘Foldon’ is derived from T4 fibritin and was described earlier as a heterologous trimerization domain (Letarov et al., (1993); S-Guthe et al., (2004)).

In preferred embodiments, the heterologous trimerization domain is a foldon domain comprising the amino acid sequence GYIPEAPRDGQAYVRKDGEWVLLSTFL (SEQ ID NO: 2).

In certain embodiments, the proteins comprise a signal peptide of an RSV F_{A} protein to improve expression of soluble protein. It will be understood by the skilled person that the processed RSV F_{B} proteins do not comprise a signal peptide.

Again, it is to be understood that according to the present invention the numbering of the positions of the amino acid residues is according to the numbering of the amino acids in SEQ ID NO: 1.

According to the invention it has been demonstrated that the presence of the specific stabilizing amino acids at the indicated positions increases the stability of the proteins in the pre-fusion conformation. According to the invention, the specific amino acids can be either already present in the amino acid sequence or can be introduced by substitution (mutation) of the amino acid on that position into the specific amino acid according to the invention.

The present invention thus provides new recombinant stabilized pre-fusion RSV F_{B} proteins, i.e. RSV F_{B} proteins that are stabilized in the pre-fusion conformation, and/or fragments thereof. The stable pre-fusion RSV F proteins of the invention, or fragments thereof, are in the pre-fusion conformation, i.e. they comprise (display) at least one epitope that is specific to the pre-fusion conformation F protein. An epitope that is specific to the pre-fusion conformation F protein is an epitope that is not present in the post-fusion conformation. Without wishing to be bound by any particular theory, it is believed that the pre-fusion conformation of RSV F_{B} protein contains epitopes that are the same as those on the RSV F_{B} protein expressed on natural RSV virions, and therefore may provide advantages for eliciting protective neutralizing antibodies.

In certain embodiments, the pre-fusion RSV F_{B} proteins of the invention, or fragments thereof, comprise at least one epitope that is recognized by a pre-fusion specific monoclonal antibody, e.g. CR9501. CR9501 comprises the heavy and light chain variable regions, and thus the binding specificities, of the antibody 58C5, which has previously been shown to be a pre-fusion specific monoclonal antibody, i.e. an antibody that binds to RSV F protein in its pre-fusion conformation and not to the post-fusion conformation (see WO2012/006596).

As indicated above, fragments of the pre-fusion RSV F protein are also encompassed by the present invention. The fragment may result from either or both of amino-terminal (e.g. by cleaving off the signal sequence) and carboxy-terminal deletions (e.g. by deleting the transmembrane region and/or cytoplasmic tail). The fragment may be chosen to comprise an immunologically active fragment of the F protein, i.e. a part that will give rise to an immune response in a subject. This can be easily determined using in silico, in vitro and/or in vivo methods, all routine to the skilled person. The term “fragment” as used herein thus refers to a protein that has an amino-terminal and/or carboxy-terminal and/or internal deletion, but where the remaining amino acid sequence is identical to the corresponding positions in the sequence of an RSV F_{B} protein, for example, the full-length sequence of a RSV F_{B} protein. It will be appreciated that for inducing an immune response and in general for vaccination purposes, a protein does not need to be full length nor have all its wild type functions, and fragments of the protein (i.e. without signal peptide) are equally useful.

In certain embodiments, the encoded proteins or fragments thereof according to the invention comprise a signal sequence, also referred to as leader sequence or signal peptide, corresponding to amino acids 1-25 of SEQ ID NO: 1. Signal sequences typically are short (e.g. 5-30 amino acids long) amino acid sequences present at the N-terminus of the majority of newly synthesized proteins that are destined towards the secretory pathway, and are typically cleaved by signal peptidase to generate a free signal peptide and a mature protein.

The signal sequence may be a signal sequence of an RSV F_{A} or an RSV F_{B} protein. In certain embodiments, the proteins or fragments thereof according to the invention do not comprise a signal sequence.

In certain embodiments, the level of expression of the pre-fusion RSV F_{B} proteins of the invention is increased, as compared to a non-stabilized wild-type RSV F_{B} protein (i.e. without the stabilizing amino acids).

In certain embodiments the pre-fusion content (defined as fraction of F_{B} protein that binds to the prefusion—specific CR9501 antibody) is significantly higher 7 days after harvest of the proteins after storage at 4° C., as compared to the F_{B} protein without said stabilizing substitutions. In certain embodiments the pre-fusion content was significantly higher 30 days after harvest of the proteins after storage at 4° C., as compared to the F_{B} protein without said stabilizing substitutions. Thus, in certain embodiments, the purified pre-fusion RSV F_{B} proteins according to the invention have an increased stability upon storage a 4° C. as compared to RSV F proteins without the stabilizing amino acid residues at the defined positions. With “stability upon storage”, it is meant that the proteins still display the at least one epitope specific for a pre-fusion specific antibody (e.g. CR9501) upon storage of the protein in solution (e.g. culture medium) at 4° C. after a certain time period. In certain embodiments, the proteins display the at least one pre-fusion specific epitope for at least 1, 2, 3, 4, 5 or 6 months, preferably for at least 1 year upon storage of the pre-fusion RSV F proteins at 4° C.

The pre-fusion RSV F_{B} proteins according to the invention are stabilized in the pre-fusion conformation by the presence of one or more of the stabilizing amino acids (either already present or introduced by mutations), i.e. do not readily change into the post-fusion conformation upon processing of the proteins, such as e.g. purification, freeze-thaw cycles, and/or storage etc.

In certain embodiments, the purified pre-fusion RSV F proteins according to the invention have an increased stability upon storage a 37° C. as compared to RSV F proteins without the stabilizing amino acid residues at the defined positions.

In certain embodiments, the pre-fusion RSV F_{B} proteins according to the invention have an increased thermostability as determined measuring the melting temperature, as described in Example 4 as compared to RSV F proteins with different (e.g. wild type) amino acid residues at the defined positions.

In certain embodiments, the proteins display a higher trimer content after being subjected to freeze-thaw conditions in appropriate formulation buffers, as compared to RSV F proteins with different (e.g. wild type) amino acid residues at the defined positions.

In certain preferred embodiments the RSV FB proteins comprise an amino acid sequence selected from the group consisting of SEQ ID NO: 14, 16, 17, 18, 29, 30, 32 and 34. It is to be understood that after expression and processing the proteins will not contain the signal peptide and p27 peptide anymore. Thus, in certain preferred embodiments, the RSV F_{B} proteins comprise an amino acid sequence comprising an F2 domain comprising the amino acids 26-109 of SEQ ID NO: 14 and an F1 domain comprising the amino acids 137-513 of SEQ ID NO: 14; an F2 domain comprising the amino acids 26-109 of SEQ ID NO: 16 and an F1 domain comprising the amino acids 137-513 of SEQ ID NO: 16; an F2 domain comprising the amino acids 26-109 of SEQ ID NO: 17 and an F1 domain comprising the amino acids 137-513 of SEQ ID NO: 17; an F2 domain comprising the amino acids 26-109 of SEQ ID NO: 18 and an F1 domain comprising the amino acids 137-513 of SEQ ID NO: 18; or an F2 domain comprising the amino acids 26-109 of SEQ ID NO: 29 and an F1 domain comprising the amino acids 137-574 of SEQ ID NO: 29, or an F2 domain comprising the amino acids 26-109 of SEQ ID NO: 32 and an F1 domain comprising the amino acids 137-574 of SEQ ID NO: 32. It is noted that the protein of SEQ ID NO: 30 and 34 will not be processed and will remain a single chain protein comprising the amino acids 26-574 of SEQ ID NO: 30 or 34.

In certain embodiments, the proteins comprise a HIS-Tag, strep-tag or c-tag. A His-Tag or polyhistidine-tag is an amino acid motif in proteins that consists of at least five histidine (H) residues; a strep-tag is an amino acid sequence that consist of 8 residues (WSHPQFEK (SEQ ID NO: 27); a c-tag is an amino acid motif that consists of 4 residues (EPEA; SEQ ID NO: 28). The tags are often at the N- or C-terminus of the protein and are generally used for purification purposes.

As described above, it is known that RSV exists as a single serotype having two antigenic subgroups: A and B. The amino acid sequences of the mature processed F protein ectodomains of the two groups are about 95% identical. As used throughout the present application, the amino acid positions are given in reference to a consensus sequence of the F protein of clinical isolates of subgroup B (SEQ ID NO: 1). As used in the present invention, the wording “the amino acid residue at position “x” of the RSV F protein thus means the amino acid corresponding to the amino acid at position “x” in the RSV F protein of SEQ ID NO: 1. Note that, in the numbering system used throughout this application 1 refers to the N-terminal amino acid of the consensus sequence of an immature F0 protein (SEQ ID NO: 1). When an F protein of another RSV B strain is used, the amino acid positions of the F protein are to be numbered with reference to the numbering of the F protein of SEQ ID NO: 1 by aligning the sequences of the other RSV B strain with the F protein consensus of SEQ ID NO: 1 with the insertion of gaps as needed. Sequence alignments can be done using methods well known in the art, e.g. by CLUSTALW, Bioedit or CLC Workbench.

As used throughout the present application nucleotide sequences are provided from 5′ to 3′ direction, and amino acid sequences from N-terminus to C-terminus, as custom in the art.

An amino acid according to the invention can be any of the twenty naturally occurring (or ‘standard’ amino acids). The standard amino acids can be divided into several groups based on their properties. Important factors are charge, hydrophilicity or hydrophobicity, size and functional groups. These properties are important for protein structure and protein-protein interactions. Some amino acids have special properties such as cysteine, that can form covalent disulfide bonds (or disulfide bridges) to other cysteine residues, proline that induces turns of the protein backbone, and glycine that is more flexible than other amino acids. Table 1 shows the abbreviations and properties of the standard amino acids.

It will be appreciated by a skilled person that the mutations can be made to the protein by routine molecular biology procedures. The mutations according to the invention preferably result in increased expression levels and/or increased stabilization of the pre-fusion RSV F_{B} proteins as compared to RSV FB proteins that do not comprise these mutation(s).

The present invention further provides nucleic acid molecules encoding the RSV F_{B} proteins according to the invention. The term “nucleic acid molecule” as used in the present invention refers to a polymeric form of nucleotides (i.e. polynucleotides) and includes both DNA (e.g. cDNA, genomic DNA) and RNA, and synthetic forms and mixed polymers of the above. It is to be understood that numerous different nucleic acid molecules can encode the same protein as a result of the degeneracy of the genetic code. It is also understood that skilled persons can, using routine techniques, make nucleotide substitutions that do not affect the protein sequence encoded by the polynucleotides described there to reflect the codon usage of any particular host organism in which the proteins are to be expressed. Therefore, unless otherwise specified, a “nucleic acid molecule encoding an amino acid sequence” includes all nucleotide sequences that are degenerate versions of each other and that encode the same amino acid sequence. Nucleotide sequences that encode proteins and RNA can include introns. Sequences herein are provided from 5′ to 3′ direction, as custom in the art.

In preferred embodiments, the nucleic acid molecules encoding the proteins according to the invention are codon-optimized for expression in mammalian cells, preferably human cells, or insect cells. Methods of codon-optimization are known and have been described previously (e.g. WO 96/09378 for mammalian cells). A sequence is considered codon-optimized if at least one non-preferred codon as compared to a wild type sequence is replaced by a codon that is more preferred. Herein, a non-preferred codon is a codon that is used less frequently in an organism than another codon coding for the same amino acid, and a codon that is more preferred is a codon that is used more frequently in an organism than a non-preferred codon. The frequency of codon usage for a specific organism can be found in codon frequency tables, such as in http://www.kazusa.or.jp/codon. Preferably more than one non-preferred codon, preferably most or all non-preferred codons, are replaced by codons that are more preferred. Preferably the most frequently used codons in an organism are used in a codon-optimized sequence. Replacement by preferred codons generally leads to higher expression.

Nucleic acid sequences can be cloned using routine molecular biology techniques, or generated de novo by DNA synthesis, which can be performed using routine procedures by service companies having business in the field of DNA synthesis and/or molecular cloning (e.g. GeneArt, GenScripts, Invitrogen, Eurofins).

In certain preferred embodiments the nucleic acids encode RSV F_{B} proteins comprising an amino acid sequence selected from the group consisting of SEQ ID NO: 14, 16, 17, 18, 29, 30, 32 and 34.

In certain preferred embodiments, the nucleic acids comprise a nucleotide sequence selected from the group consisting of SEQ ID NO: 31 and 33.

The invention also provides vectors comprising a nucleic acid molecule as described above. In certain embodiments, a nucleic acid molecule according to the invention thus is part of a vector. Such vectors can easily be manipulated by methods well known to the person skilled in the art and can for instance be designed for being capable of replication in prokaryotic and/or eukaryotic cells. The vector used can be any vector that is suitable for cloning DNA and that can be used for expression of a nucleic acid molecule of interest. Suitable vectors according to the invention are e.g. adenovectors, alphavirus, paramyxovirus, vaccinia virus, herpes virus, retroviral vectors etc.

In certain embodiments of the invention, the vector is an adenovirus vector. An adenovirus according to the invention belongs to the family of the Adenoviridae, and preferably is one that belongs to the genus Mastadenovirus. It can be a human adenovirus, but also an adenovirus that infects other species, including but not limited to a bovine adenovirus (e.g. bovine adenovirus 3, BAdV3), a canine adenovirus (e.g. CAdV2), a porcine adenovirus (e.g. PAdV3 or 5), or a simian adenovirus (which includes a monkey adenovirus and an ape adenovirus, such as a chimpanzee adenovirus or a gorilla adenovirus). Preferably, the adenovirus is a human adenovirus (HAdV, or AdHu), or a simian adenovirus such as chimpanzee or gorilla adenovirus (ChAd, AdCh, or SAdV), or a rhesus monkey adenovirus (RhAd). In the invention, a human adenovirus is meant if referred to as Ad without indication of species, e.g. the brief notation “Ad26” means the same as HAdV26, which is human adenovirus serotype 26. Also as used herein, the notation “rAd” means recombinant adenovirus, e.g., “rAd26” refers to recombinant human adenovirus 26.

Most advanced studies have been performed using human adenoviruses, and human adenoviruses are preferred according to certain aspects of the invention. In certain preferred embodiments, a recombinant adenovirus according to the invention is based upon a human adenovirus. In preferred embodiments, the recombinant adenovirus is based upon a human adenovirus serotype 5, 11, 26, 34, 35, 48, 49, 50, 52, etc. According to a particularly preferred embodiment of the invention, an adenovirus is a human adenovirus of serotype 26. Advantages of these serotypes include a low seroprevalence and/or low pre-existing neutralizing antibody titers in the human population, and experience with use in human subjects in clinical trials.

Simian adenoviruses generally also have a low seroprevalence and/or low pre-existing neutralizing antibody titers in the human population, and a significant amount of work has been reported using chimpanzee adenovirus vectors (e.g. U.S. Pat. No. 6,083,716; WO 2005/071093; WO 2010/086189; WO 2010085984; Farina et al, 2001, J Virol 75: 11603-13; Cohen et al, 2002, J Gen Virol 83: 151-55; Kobinger et al, 2006, Virology 346: 394-401; Tatsis et al., 2007, Molecular Therapy 15: 608-17; see also review by Bangari and Mittal, 2006, Vaccine 24: 849-62; and review by Lasaro and Ertl, 2009, Mol Ther 17: 1333-39). Hence, in other embodiments, the recombinant adenovirus according to the invention is based upon a simian adenovirus, e.g. a chimpanzee adenovirus. In certain embodiments, the recombinant adenovirus is based upon simian adenovirus type 1, 7, 8, 21, 22, 23, 24, 25, 26, 27.1, 28.1, 29, 30, 31.1, 32, 33, 34, 35.1, 36, 37.2, 39, 40.1, 41.1, 42.1, 43, 44, 45, 46, 48, 49, 50 or SA7P. In certain embodiments, the recombinant adenovirus is based upon a chimpanzee adenovirus such as ChAdOx 1 (see e.g. WO 2012/172277), or ChAdOx 2 (see e.g. WO 2018/215766). In certain embodiments, the recombinant adenovirus is based upon a chimpanzee adenovirus such as BZ28 (see e.g. WO 2019/086466). In certain embodiments, the recombinant adenovirus is based upon a gorilla adenovirus such as BLY6 (see e.g. WO 2019/086456), or BZ1 (see e.g. WO 2019/086466).

In a preferred embodiment of the invention, the adenoviral vectors comprise capsid proteins from rare serotypes, e.g. including Ad26. In the typical embodiment, the vector is an rAd26 virus. An “adenovirus capsid protein” refers to a protein on the capsid of an adenovirus (e.g., Ad26, Ad35, rAd48, rAd5HVR48 vectors) that is involved in determining the serotype and/or tropism of a particular adenovirus. Adenoviral capsid proteins typically include the fiber, penton and/or hexon proteins. As used herein a “capsid protein” for a particular adenovirus, such as an “Ad26 capsid protein” can be, for example, a chimeric capsid protein that includes at least a part of an Ad26 capsid protein. In certain embodiments, the capsid protein is an entire capsid protein of Ad26. In certain embodiments, the hexon, penton and fiber are of Ad26.

One of ordinary skill in the art will recognize that elements derived from multiple serotypes can be combined in a single recombinant adenovirus vector. Thus, a chimeric adenovirus that combines desirable properties from different serotypes can be produced. Thus, in some embodiments, a chimeric adenovirus of the invention could combine the absence of pre-existing immunity of a first serotype with characteristics such as temperature stability, assembly, anchoring, production yield, redirected or improved infection, stability of the DNA in the target cell, and the like. See for example WO 2006/040330 for chimeric adenovirus Ad5HVR48, that includes an Ad5 backbone having partial capsids from Ad48, and also e.g. WO 2019/086461 for chimeric adenoviruses Ad26HVRPtr1, Ad26HVRPtr12, and Ad26HVRPtr13, that include an Ad26 virus backbone having partial capsid proteins of Ptr1, Ptr12, and Ptr13, respectively)

In certain preferred embodiments the recombinant adenovirus vector useful in the invention is derived mainly or entirely from Ad26 (i.e., the vector is rAd26). In some embodiments, the adenovirus is replication deficient, e.g., because it contains a deletion in the E1 region of the genome. For adenoviruses being derived from non-group C adenovirus, such as Ad26 or Ad35, it is typical to exchange the E4-orf6 coding sequence of the adenovirus with the E4-orf6 of an adenovirus of human subgroup C such as Ad5. This allows propagation of such adenoviruses in well-known complementing cell lines that express the E1 genes of Ad5, such as for example 293 cells, PER.C6 cells, and the like (see, e.g. Havenga, et al., 2006, J Gen Virol 87: 2135-43; WO 03/104467). However, such adenoviruses will not be capable of replicating in non-complementing cells that do not express the E1 genes of Ad5.

The preparation of recombinant adenoviral vectors is well known in the art. Preparation of rAd26 vectors is described, for example, in WO 2007/104792 and in Abbink et al., (2007) Virol 81(9): 4654-63. Exemplary genome sequences of Ad26 are found in GenBank Accession EF 153474 and in SEQ ID NO: 1 of WO 2007/104792. Examples of vectors useful for the invention for instance include those described in WO2012/082918, the disclosure of which is incorporated herein by reference in its entirety.

Typically, a vector useful in the invention is produced using a nucleic acid comprising the entire recombinant adenoviral genome (e.g., a plasmid, cosmid, or baculovirus vector). Thus, the invention also provides isolated nucleic acid molecules that encode the adenoviral vectors of the invention. The nucleic acid molecules of the invention can be in the form of RNA or in the form of DNA obtained by cloning or produced synthetically. The DNA can be double-stranded or single-stranded.

The adenovirus vectors useful in the invention are typically replication deficient. In these embodiments, the virus is rendered replication deficient by deletion or inactivation of regions critical to replication of the virus, such as the E1 region. The regions can be substantially deleted or inactivated by, for example, inserting a gene of interest, such as a gene encoding the RSV F protein (usually linked to a promoter), or a gene encoding an RSV F protein (usually linked to a promoter) within the region. In some embodiments, the vectors of the invention can contain deletions in other regions, such as the E2, E3 or E4 regions, or insertions of heterologous genes linked to a promoter within one or more of these regions. For E2- and/or E4-mutated adenoviruses, generally E2- and/or E4-complementing cell lines are used to generate recombinant adenoviruses. Mutations in the E3 region of the adenovirus need not be complemented by the cell line, since E3 is not required for replication.

A packaging cell line is typically used to produce sufficient amounts of adenovirus vectors for use in the invention. A packaging cell is a cell that comprises those genes that have been deleted or inactivated in a replication deficient vector, thus allowing the virus to replicate in the cell. Suitable packaging cell lines for adenoviruses with a deletion in the E1 region include, for example, PER.C6, 911, 293, and E1 A549.

In a preferred embodiment of the invention, the vector is an adenovirus vector, and more preferably a rAd26 vector, most preferably a rAd26 vector with at least a deletion in the E1 region of the adenoviral genome, e.g. such as that described in Abbink, J Virol, 2007. 81(9): p. 4654-63, which is incorporated herein by reference. Typically, the nucleic acid sequence encoding the RSV F protein is cloned into the E1 and/or the E3 region of the adenoviral genome.

Host cells comprising the nucleic acid molecules encoding the pre-fusion RSV F_{B} proteins form also part of the invention. The pre-fusion RSV F proteins may be produced through recombinant DNA technology involving expression of the molecules in host cells, e.g. Chinese hamster ovary (CHO) cells, tumor cell lines, BHK cells, human cell lines such as HEK293 cells, PER.C6 cells, or yeast, fungi, insect cells, and the like, or transgenic animals or plants. In certain embodiments, the cells are from a multicellular organism, in certain embodiments they are of vertebrate or invertebrate origin. In certain embodiments, the cells are mammalian cells. In certain embodiments, the cells are human cells. In general, the production of a recombinant proteins, such the pre-fusion RSV F proteins of the invention, in a host cell comprises the introduction of a heterologous nucleic acid molecule encoding the RSV F protein in expressible format into the host cell, culturing the cells under conditions conducive to expression of the nucleic acid molecule and allowing expression of the protein in said cell. The nucleic acid molecule encoding an RSV F protein in expressible format may be in the form of an expression cassette, and usually requires sequences capable of bringing about expression of the nucleic acid, such as enhancer(s), promoter, polyadenylation signal, and the like. The person skilled in the art is aware that various promoters can be used to obtain expression of a gene in host cells. Promoters can be constitutive or regulated, and can be obtained from various sources, including viruses, prokaryotic, or eukaryotic sources, or artificially designed.

Cell culture media are available from various vendors, and a suitable medium can be routinely chosen for a host cell to express the protein of interest, here the pre-fusion RSV F proteins. The suitable medium may or may not contain serum. A “heterologous nucleic acid molecule” (also referred to herein as ‘transgene’) is a nucleic acid molecule that is not naturally present in the host cell. It is introduced into for instance a vector by standard molecular biology techniques. A transgene is generally operably linked to expression control sequences. This can for instance be done by placing the nucleic acid encoding the transgene(s) under the control of a promoter. Further regulatory sequences may be added. Many promoters can be used for expression of a transgene(s), and are known to the skilled person, e.g. these may comprise viral, mammalian, synthetic promoters, and the like. A non-limiting example of a suitable promoter for obtaining expression in eukaryotic cells is a CMV-promoter (U.S. Pat. No. 5,385,839), e.g. the CMV immediate early promoter, for instance comprising nt. −735 to +95 from the CMV immediate early gene enhancer/promoter. A polyadenylation signal, for example the bovine growth hormone polyA signal (U.S. Pat. No. 5,122,458), may be present behind the transgene(s). Alternatively, several widely used expression vectors are available in the art and from commercial sources, e.g. the pcDNA and pEF vector series of Invitrogen, pMSCV and pTK-Hyg from BD Sciences, pCMV-Script from Stratagene, etc, which can be used to recombinantly express the protein of interest, or to obtain suitable promoters and/or transcription terminator sequences, polyA sequences, and the like.

The cell culture can be any type of cell culture, including adherent cell culture, e.g. cells attached to the surface of a culture vessel or to microcarriers, as well as suspension culture. Most large-scale suspension cultures are operated as batch or fed-batch processes because they are the most straightforward to operate and scale up. Nowadays, continuous processes based on perfusion principles are becoming more common and are also suitable. Suitable culture media are also well known to the skilled person and can generally be obtained from commercial sources in large quantities, or custom-made according to standard protocols. Culturing can be done for instance in dishes, roller bottles or in bioreactors, using batch, fed-batch, continuous systems and the like. Suitable conditions for culturing cells are known (see e.g. Tissue Culture, Academic Press, Kruse and Paterson, editors (1973), and R. I. Freshney, Culture of animal cells: A manual of basic technique, fourth edition (Wiley-Liss Inc., 2000, ISBN 0-471-34889-9)).

The invention further provides compositions comprising a nucleic acid molecule, a protein, fragment thereof, and/or vector according to the invention. In certain embodiments, the invention provides compositions comprising a pre-fusion RSV F protein that displays an epitope that is present in a pre-fusion conformation of the RSV F protein but is absent in the post-fusion conformation, and/or a fragment thereof. The invention also provides compositions comprising a nucleic acid molecule and/or a vector, encoding such pre-fusion RSV F_{B} protein and/or vector thereof. In a preferred embodiment, the compositions comprise an RSV F_{B} protein, and/or fragment, and a vector according to the invention for concurrent administration. For administering to humans, the invention may employ pharmaceutical compositions comprising the nucleic acid, a protein, and/or vector and a pharmaceutically acceptable carrier or excipient. In the present context, the term “pharmaceutically acceptable” means that the carrier or excipient, at the dosages and concentrations employed, will not cause any unwanted or harmful effects in the subjects to which they are administered. Such pharmaceutically acceptable carriers and excipients are well known in the art (see Remington's Pharmaceutical Sciences, 18th edition, A. R. Gennaro, Ed., Mack Publishing Company [1990]; Pharmaceutical Formulation Development of Peptides and Proteins, S. Frokjaer and L. Hovgaard, Eds., Taylor & Francis [2000]; and Handbook of Pharmaceutical Excipients, 3rd edition, A. Kibbe, Ed., Pharmaceutical Press [2000]). The purified nucleic acid, a protein, and/or vector preferably is formulated and administered as a sterile solution although it is also possible to utilize lyophilized preparations. Sterile solutions are prepared by sterile filtration or by other methods known per se in the art. The solutions are then lyophilized or filled into pharmaceutical dosage containers. The pH of the solution generally is in the range of pH 3.0 to 9.5, preferably in the range of pH 5.0 to 7.5. The nucleic acid, a protein, and/or vector typically is in a solution having a suitable pharmaceutically acceptable buffer, and the solution may also contain a salt. Optionally stabilizing agent may be present, such as albumin. In certain embodiments, detergent is added. In certain embodiments, nucleic acid, a protein, and/or vector may be formulated into an injectable preparation. These formulations contain effective amounts of nucleic acid, a protein, and/or vector, are either sterile liquid solutions, liquid suspensions or lyophilized versions and optionally contain stabilizers or excipients.

For instance, adenovirus may be stored in the buffer that is also used for the Adenovirus World Standard (Hoganson et al, Development of a stable adenoviral vector formulation, Bioprocessing March 2002, p. 43-48): 20 mM Tris pH 8, 25 mM NaCl, 2.5% glycerol. Another useful formulation buffer suitable for administration to humans is 20 mM Tris, 2 mM MgCl2, 25 mM NaCl, sucrose 10% w/v, polysorbate-80 0.02% w/v. Obviously, many other buffers can be used, and several examples of suitable formulations for the storage and for pharmaceutical administration of purified (adeno)virus preparations can for instance be found in European patent no. 0853660, U.S. Pat. No. 6,225,289 and in international patent applications WO 99/41416, WO 99/12568, WO 00/29024, WO 01/66137, WO 03/049763, WO 03/078592, WO 03/061708.

In certain embodiments, the compositions may further comprise one or more adjuvants. Adjuvants are known in the art to further increase the immune response to an applied antigenic determinant, and pharmaceutical compositions comprising adenovirus and suitable adjuvants are for instance disclosed in WO 2007/110409, incorporated by reference herein. The terms “adjuvant” and “immune stimulant” are used interchangeably and are defined as one or more substances that cause stimulation of the immune system. In this context, an adjuvant is used to enhance an immune response to the adenovirus vectors of the invention. Examples of suitable adjuvants include aluminum salts such as aluminum hydroxide and/or aluminium phosphate; oil-emulsion compositions (or oil-in-water compositions), including squalene-water emulsions, such as MF59 (see e.g. WO 90/14837); saponin formulations, such as for example QS21 and Immunostimulating Complexes (ISCOMS) (see e.g. U.S. Pat. No. 5,057,540; WO 90/03184, WO 96/11711, WO 2004/004762, WO 2005/002620); bacterial or microbial derivatives, examples of which are monophosphoryl lipid A (MPL), 3-O-deacylated MPL (3dMPL), CpG-motif containing oligonucleotides, ADP-ribosylating bacterial toxins or mutants thereof, such as E. coli heat labile enterotoxin LT, cholera toxin CT, and the like. It is also possible to use vector-encoded adjuvant, e.g. by using heterologous nucleic acid that encodes a fusion of the oligomerization domain of C4-binding protein (C4bp) to the antigen of interest (e.g. Solabomi et al, 2008, Infect Immun 76: 3817-23). In certain embodiments the compositions of the invention comprise aluminium as an adjuvant, e.g. in the form of aluminium hydroxide, aluminium phosphate, aluminium potassium phosphate, or combinations thereof, in concentrations of 0.05-5 mg, e.g. from 0.075-1.0 mg, of aluminium content per dose.

In other embodiments, the compositions do not comprise adjuvants.

The invention further provides a vaccine against RSV comprising a composition as described herein.

The invention also provides the use of a stabilized pre-fusion RSV F_{B} protein, fragment thereof, a nucleic acid molecule, and/or a vector, according to the invention, for inducing an immune response against RSV F protein in a subject.

Further provided are methods for inducing an immune response against RSV F protein in a subject, in particular RSV F_{B}, comprising administering to the subject a pre-fusion RSV F_{B} protein, and/or a nucleic acid molecule, and/or a vector, according to the invention. Also provided are pre-fusion RSV _{B}F proteins, nucleic acid molecules, and/or vectors, according to the invention for use in inducing an immune response against RSV F protein, in particular RSV F_{B}, in a subject. Further provided is the use of the pre-fusion RSV F_{B} proteins, and/or nucleic acid molecules, and/or vectors according to the invention for the manufacture of a medicament for use in inducing an immune response against RSV F protein, in particular RSV F_{B}, in a subject.

Further provided are methods for vaccinating a subject against RSV, in particular against an RSV B strain, the method comprising administering to the subject a composition or vaccine as described herein.

The invention also provides methods for preventing infection and/or replication of RSV, in particular against an RSV B strain, in a subject, comprising administering to the subject a composition or vaccine as described herein.

The pre-fusion RSV F_{B} proteins, fragments, nucleic acid molecules, or vectors of the invention may be used for prevention (prophylaxis) and/or treatment of RSV infections, in particular RSV infections caused by an RSV B strain. In certain embodiments, the prevention and/or treatment may be targeted at patient groups that are susceptible for RSV infection.

Such patient groups include, but are not limited to e.g., the elderly (e.g. ≥50 years old, ≥60 years old, and preferably ≥65 years old), the young (e.g. ≤5 years old, ≤1 year old), pregnant women (for maternal immunization), hospitalized patients and patients who have been treated with an antiviral compound but have shown an inadequate antiviral response.

The pre-fusion RSV F_{B} proteins, fragments, nucleic acid molecules and/or vectors according to the invention may be used e.g. in stand-alone treatment and/or prophylaxis of a disease or condition caused by RSV, or in combination with other prophylactic and/or therapeutic treatments, such as (existing or future) vaccines, antiviral agents and/or monoclonal antibodies.

The invention further provides methods for preventing and/or treating RSV infection, in particular an RSV infection caused by an RSV B strain, in a subject in need thereof, utilizing the pre-fusion RSV F_{B} proteins, fragments, nucleic acid molecules and/or vectors according to the invention.

In a specific embodiment, said methods for preventing and/or treating RSV infection, in particular an RSV infection caused by an RSV B strain, in a subject comprises administering to a subject in need thereof an effective amount of a pre-fusion RSV F_{B} protein, fragment, nucleic acid molecule and/or a vector, as described herein. A therapeutically effective amount refers to an amount of a protein, nucleic acid molecule or vector, that is effective for preventing, ameliorating and/or treating a disease or condition resulting from infection by RSV. Prevention encompasses inhibiting or reducing the spread of RSV or inhibiting or reducing the onset, development or progression of one or more of the symptoms associated with infection by RSV. Amelioration as used in herein may refer to the reduction of visible or perceptible disease symptoms, viremia, or any other measurable manifestation of influenza infection.

In certain embodiments, said methods result in the prevention of reverse transcriptase polymerase chain reaction (RT PCR)-confirmed RSV-mediated lower respiratory tract disease (LRTD). In certain embodiments, said methods result in the reduction of reverse transcriptase polymerase chain reaction (RT PCR)-confirmed RSV-mediated lower respiratory tract disease (LRTD), as compared to subjects which have not been administered the vaccine combination.

In addition, or alternatively, said methods are characterized by an absent or reduced RSV viral load in the nasal track and/or lungs of the subject upon exposure to RSV.

In addition, or alternatively, said methods are characterized by an absent or reduced RSV clinical symptom in the subject upon exposure to RSV.

In addition, or alternatively, said methods are characterized by the presence of neutralizing antibodies to RSV and/or protective immunity against RSV, in particular an RSV B strain.

In certain preferred embodiments, the methods have an acceptable safety profile.

In certain embodiments, the invention provides methods for making a vaccine against respiratory syncytial virus (RSV), in particular against RSV B, comprising providing an RSV F_{B} protein, fragment, nucleic acid or vector according to the invention and formulating it into a pharmaceutically acceptable composition.

According to the invention, the term “vaccine” refers to an agent or composition containing an active component effective to induce a certain degree of immunity in a subject against a certain pathogen or disease, which will result in at least a decrease (up to complete absence) of the severity, duration or other manifestation of symptoms associated with infection by the pathogen or the disease. In the present invention, the vaccine comprises an effective amount of a pre-fusion RSV F_{B} protein, fragment, a nucleic acid molecule encoding the pre-fusion RSV F_{B} protein, and/or a vector comprising said nucleic acid molecule, which results in an immune response against the F protein of RSV. This provides a method of preventing serious lower respiratory tract disease leading to hospitalization and the decrease in frequency of complications such as pneumonia and bronchiolitis due to RSV infection and replication in a subject. The term “vaccine” according to the invention implies that it is a pharmaceutical composition, and thus typically includes a pharmaceutically acceptable diluent, carrier or excipient. It may or may not comprise further active ingredients. In certain embodiments it may be a combination vaccine that further comprises other components that induce an immune response, e.g. against other proteins of RSV and/or against other infectious agents. The administration of further active components may for instance be done by separate administration or by administering combination products of the vaccines of the invention and the further active components.

Compositions may be administered to a subject, e.g. a human subject. The total dose of the RSV F_{B} proteins in a composition for a single administration can for instance be about 0.01 μg to about 10 mg, e.g. 1 μg-1 mg, e.g. 10 μg-100 μg. The total dose of the (adeno)vectors comprising DNA encoding the RSV F proteins in a composition for a single administration can for instance be about 0.1×10¹⁰ vp/ml and 2×10^{11 ,} preferably between about 1×10¹⁰ vp/ml and 2×10¹¹ vp/ml, preferably between 5×10¹⁰ vp/ml and 1×10¹¹ vp/ml.

Administration of the compositions according to the invention can be performed using standard routes of administration. Non-limiting embodiments include parenteral administration, such as intradermal, intramuscular, subcutaneous, transcutaneous, or mucosal administration, e.g. intranasal, oral, and the like. In one embodiment a composition is administered by intramuscular injection. The skilled person knows the various possibilities to administer a composition, e.g. a vaccine in order to induce an immune response to the antigen(s) in the vaccine.

A subject as used herein preferably is a mammal, for instance a rodent, e.g. a mouse, a cotton rat, or a non-human-primate, or a human. Preferably, the subject is a human subject.

The proteins, nucleic acid molecules, vectors, and/or compositions may also be administered, either as prime, or as boost, in a homologous or heterologous prime-boost regimen. If a boosting vaccination is performed, typically, such a boosting vaccination will be administered to the same subject at a time between one week and one year, preferably between two weeks and four months, after administering the composition to the subject for the first time (which is in such cases referred to as ‘priming vaccination’). In certain embodiments, the administration comprises a prime and at least one booster administration.

In addition, the proteins of the invention may be used as diagnostic tool, for example to test the immune status of an individual by establishing whether there are antibodies in the serum of such individual capable of binding to the protein of the invention. The invention thus also relates to an in vitro diagnostic method for detecting the presence of an RSV infection in a patient said method comprising the steps of a) contacting a biological sample obtained from said patient with a protein according to the invention; and b) detecting the presence of antibody-protein complexes.

### EXAMPLES

### Example 1: Design of a Soluble Trimeric Protein by the Introduction of a C-Terminal Foldon and Stabilizing Point Mutations

Several pre-fusion RSV F protein variants were produced. The soluble candidates are truncated at amino acid position 513 of RSV B F1 domain and fused with a four amino acid linker (SAIG) to a fibritin trimerization domain (foldon) (GYIPEAPRDGQAYVRKDGEWVLLSTFL; SEQ ID NO: 2). An RSV B signal peptide (SEQ ID NO: 24) or RSV A signal peptide (SEQ ID NO: 23) of the fusion protein were used for expression of the proteins. Some of the designs had a linker and C-tag, C-terminal to the foldon sequence to allow affinity purification (e.g. SEQ ID NO 3).

To stabilize the prefusion conformation of the proteins several combinations of point mutations were introduced, e.g. one or more of the mutations P101Q, I152M, K226M, D486N, S215P, L2031, and/or D489Y.

### Example 2: Expression and Stability of RSV B F Variants After Transient Transfection in HEK293F Cells

The RSV F non-stabilized protein used as a control for expression and stability (SEQ ID NO: 3) was based on a truncated consensus sequence for subgroup B (SEQ ID NO: 1) and comprises the ectodomain of the RSV F_{B} protein of SEQ ID NO: 1 containing a C-terminal fusion, through a linker, with a foldon domain (SEQ ID NO: 2) and an N-terminal signal peptide based on RSV F type A (SEQ ID NO: 23). To allow affinity purification a C-tag was introduced for selected designs.

DNA fragments encoding the proteins of the invention were synthesized (Genscript) and cloned in the pcDNA2004 expression vector (modified pcDNA3 plasmid with an enhanced CMV promotor). The expression platform used was the 293Freestyle cells (Life Technologies) in 24-deep well plates. The cells were transiently transfected using 293Fectin (Life Technologies) according to the manufacturer's instructions and cultured for 5 days at 37° C. and 10% CO₂. For RSV180910 (SEQ ID NO: 7), RSV180916 (SEQ ID NO: 8), RSV180907 (SEQ ID NO: 13), RSV180913 (SEQ ID NO: 14), RSV190417 (SEQ ID NO: 15), RSV190414 (SEQ ID NO: 16) and RSV190420 (SEQ ID NO: 17) in FIG. 2B and FIG. 2C cells were co-transfected with a 9:1 ratio of RSV F plasmid and furin plasmid to increase furin cleavage efficiency. The culture supernatant was harvested and spun for 5 minutes at 300 g to remove cells and cellular debris. The spun supernatant was subsequently sterile filtered using a 0.22 μm vacuum filter and stored at 4° C. until use.

Quantitative Octet (BioLayer Interferometry) was used for measuring protein concentration in the supernatants at day of harvest and after 7 or 30 days storage at 4° C. CR9501 (an antibody specifically recognizing pre-fusion RSV F protein, which comprises the variable regions of the antibodies 58C5 as described in WO2012/006596) and CR9506 (recognizing pre-fusion and post-fusion RSV F protein and comprising a heavy chain variable region comprising SEQ ID NO: 21, and a light chain variable region comprising SEQ ID NO: 22) were biotinylated by standard protocols and immobilized on Streptavidin biosensors (ForteBio, Portsmouth, UK). For the post-fusion specific antibody ADI-15644 (Gilman et al., 2016) anti-human Fc sensors were used to immobilize the antibody on the biosensors.

Afterwards, the coated biosensors were blocked in mock cell culture supernatant. A quantitative experiment was performed as follows: temperature 30° C., shaking speed 1000 rpm, time of the assay 300 sec. The concentration of the protein was calculated using a standard curve. The standard curve was prepared for each coated antibody CR9501 and CR9506 using the pre-fusion RSV F_{A} protein (SEQ ID NO: 19; described previously in WO17174568) diluted in mock medium. For ADI-15644 the binding at equilibrium (2A) or initial binding rate per second was defined (2C). In FIG. 2C a positive control for anti RSV postF binding was taken along, therefore 201.tg RSV postF protein (SEQ ID NO: 20) was spiked into supernatant of mock-transfected cells and measured. The data analysis was done using the ForteBio Data Analysis 10.0.1.6 software (ForteBio).

### Results and Discussion:

All variants showed F expression (FIG. 2) as measured by CR9506 binding. Non-stabilized RSV F type B (RSV181177, SEQ ID NO: 3) showed very low expression of pre-fusion protein at the day of harvest, as measured by Mab CR9501 binding, and this pre-fusion F protein was also highly unstable based on the loss of binding to the CR9501 after storage of the supernatant at 4° C. for 7 days (FIG. 2A). Additionally, relative higher amount of post-fusion F protein was detected with Mab ADI-15644 for the non-stabilized F. The total amount of polypeptide in supernatant and the amount of pre-fusion polypeptide could be increased by stabilizing mutations I152M and K226M (RSV181178 (SEQ ID NO: 4)) (FIG. 2A). The stability in supernatant for 7 days was further improved by stabilizing mutation D486N (RSV181179; SEQ ID NO: 5) and S215P (RSV180915; SEQ ID NO: 6). Subsequent addition of stabilizing mutations L2031 and P101Q increased expression levels further and reduced amounts of post-fusion polypeptide to a level that is hardly detectable (RSV180916; SEQ ID NO: 8) (FIG. 2A). Introduction of stabilizing mutations D489Y, T357R and N371Y (RSV180917 and SEQ ID NO: 9) decreased expression levels. When subsequently P101Q (RSV181180 (SEQ ID NO: 10) and D489Y (RSV181181 (SEQ ID NO: 11) and T357R+N371Y (RSV181182 (SEQ ID NO: 12) were added to the stabilized F variant with I152M, K226M, D486N and S215P F variant (RSV180915), no increase in expression levels were observed but the stability did increase since no post-fusion F could be detected after 7 days of storage at 4° C.

Next, the effect of the signal peptide on RSV F expression was evaluated by comparing an RSV F type A signal peptide (SEQ ID NO: 23) with an RSV F type B signal peptide (SEQ ID NO: 24). In RSV F_{B} variants with or without C-terminal C-tag, expression levels (CR9506 binding) and pre-fusion content (CR9501 binding) was higher when an RSV F type A signal peptide was used for expression (e.g. as in SEQ ID NO: 8 and 14) (FIG. 2B).

Variants described in FIG. 2C are without a tag. Variant RSV1800913 (SEQ ID NO: 14) with stabilizing mutations I152M, K226M, D486N, S215P, L2031 and P101Q showed high binding to pre-fusion specific Mab CR9501 and no trace of post-fusion F was detected at day of harvest. After 30 days storage at 4° C. the pre-fusion levels did not decrease.

The introduction of D489Y (RSV190417; SEQ ID NO: 15) remains similar to RSV180913. Subsequent backmutation to consensus K226 (RSV190414: SEQ ID NO: 16) show slightly reduced expression in supernatant. These variants were further investigated after purification (Example 4). The drift mutations L172Q and S173L (RSV190420 (SEQ ID NO: 17)) did not impact expression and pre-fusion content and this variant was further investigated after purification (Example 4). After storage of the supernatant for 30days at 4° C. no postF binding was measurable.

### Example 3: Production and Purification of Selected Variants

DNA fragments encoding the polypeptides of the invention were synthesized (Genscript) and cloned in the pcDNA2004 expression vector (in-house modified pcDNA3 plasmid with an enhanced CMV promotor).

HEK293 cells were used as expression platform for RSV180915 (SEQ ID NO: 6), RSV180916 (SEQ ID NO: 8), RSV180917 (SEQ ID NO: 9), RSV181180 (SEQ ID NO: 10), RSV181181 (SEQ ID NO: 11), RSV181182 (SEQ ID NO: 12), RSV190414 (SEQ ID NO: 16), RSV190420 (SEQ ID NO: 17) and RSV200125 (SEQ ID NO: 18). RSV190414 (SEQ ID NO: 16), RSV190420 (SEQ ID NO: 17) and RSV200125 (SEQ ID NO: 18) were co-transfected with 10% furin coding plasmid to increase previously observed incomplete processing.

The cells were transiently transfected using 293Fectin (Life Technologies) according to the manufacturer's instructions and cultured for 5 days at 37° C. and 10% CO2. The culture supernatant was harvested and spun for 5 minutes at 300 g to remove cells and cellular debris. The spun supernatant was subsequently sterile filtered using a 0.22 μm vacuum filter and stored at 4° C. until use.

The proteins were purified using a two-step purification protocol including either CaptureSelectTM C-tag affinity column for C-tagged polypeptides RSV180915 (SEQ ID NO: 6), RSV180916 (SEQ ID NO: 8), RSV180917 (SEQ ID NO: 9), RSV181180 (SEQ ID NO: 10), RSV181181 (SEQ ID NO: 11) and RSV181182 (SEQ ID NO: 12) or, for the non-tagged proteins RSV190414 (SEQ ID NO: 16), RSV190420 (SEQ ID NO: 17) and RSV200125 (SEQ ID NO: 18) by cation-exchange at pH 5.0 (HiTrap Capto SP ImpRes column; GE Life Sciences, Pittsburgh, PA, USA). All proteins were further purified by size-exclusion chromatography using a Superdex 200 column (GE Life Sciences, Pittsburgh, PA, USA).

HEK293E 253 cells were used as expression platform for RSV180913 (SEQ ID NO: 14) on large scale. After 6 days the medium containing the protein was harvested by low-speed centrifugation (10 minutes, 1000 g) followed by high-speed centrifugation (10 minutes, 4000g). The conditioned medium was concentrated using a 30 kDa Quixstand hollow fiber cardridge. Next, the concentrated medium was diafiltrated against 1 L PBS and 1 L 20 mM NaOAc, 100 mM NaCl, pH 5.0. Aggregates were removed by centrifugation and the concentrated, diafiltrated medium was diluted 1:1 with buffer 20 mM NaOAc, pH 5.0. Next, pre-fusion F proteins were further purified using Cation-exchange at pH 5.0 (10 ml Capto SP-Impres in a XK16 column (GE Life Sciences, Pittsburgh, PA, USA), followed by anion exchange chromatography using a Resource-Q column (GE Life Sciences, Pittsburgh, PA, USA) at pH 8. Finally, the protein was purified further by gel filtration using a Superdex200 16/600 column (GE Life Sciences, Pittsburgh, PA, USA).

### Results and Discussion:

Several stabilized RSV prefusion F type B variants (RSV F_{B} proteins) were purified by ion exchange followed by SEC (FIG. 3). The main peak at about 11.5-13 ml volumes for RSV180915, RSV180916 and RSV180917, 65m1 volumes for RSV190420, RSV180913 and RSV200125 corresponds to the trimeric RSV pre-fusion F_{B} protein.

### Example 4: Characterization and Stability of preF Type B Trimers

### SDS-PAGE Analysis and Western Blot

Selected representative purified proteins from Example 3 were analyzed on 4-12% (w/v) Bis-Tris NuPAGE gels, 1×MOPS (Life Technologies) under reducing and non-reducing conditions Coomassie stained. For the F variants that were transfected without furin co-transfection (RSV180915, RSV180916 and RSV180917), Western Blot analysis was performed as follows: Semi-dry blotting performed according to manufacturers' recommendations. Blocking for 1 hr in 5% blotting grade blocker in TBS-Tween (5% blotting grade blocker (BGB)), 1^{st} antibody (CR9506 1:10.000 in 5% BGB) incubation o/n, 2^{nd} antibody (α-human IgG CW800 (Rockland Immunochemicals, Inc., Limerick, PA, US) 1:5000 in 5%) incubation for 1 hr. All incubations were performed on a roller platform at room temperature. After first and second antibody, the blots were washed 3× using 10 ml TBS/0.05% Tween20 for each wash, for 5 min, followed by a final wash using 10 ml of PBS. The blots were visualized by scanning on an Odyssey scanner, using both the 700CW and 800CW channel. Scanning intensity for the 700CW and 800CW channel was set at 5. Scanning quality is set at medium.

### Results and Discussion:

In FIG. 4A incomplete processing into F1 and F2 was detected for proteins that were produced in 293HEK cells without furin co-transfection. Respective band is indicated with F1+p27 in FIG. 4A. Purified proteins obtained after co-transfection with furin showed a single band at the expected height of F1 and F1+F2 ectodomain for reduced and non-reduced gels respectively (FIG. 4B and C).

Trimer Content of RSV preF Type B Proteins

The purified RSV pre-fusion F_{B} proteins were analyzed on analytical SEC to confirm the purity and trimeric nature.

For RSV180915 (SEQ ID NO: 6), RSV180916 (SEQ ID NO: 8), RSV180917 (SEQ ID NO: 9), RSV181180 (SEQ ID NO: 10), RSV181181 (SEQ ID NO: 11) and RSV181182 (SEQ ID NO: 12) the analysis was performed using a High Performance Liquid Chromatography (HPLC) Infinity 1260 series setup (Agilent). Of each purified protein 40 μg was run (1 mL/min.) over a TSK gel G3000SW×1 column (Sigma-Aldrich). The elution was monitored by a UV detector (Thermo Fisher Scientific), a μDawn Light Scatter (LS) detector (Wyatt Technologies), a μT-rEx Refractive Index (RI) detector (Wyatt Technologies) and a Nanostar Dynamic Light Scattering (DLS) detector (Wyatt Technologies). The trimeric protein has a retention time of about 6.5 minutes. The SEC profiles were analyzed by the Astra 7.3.2.19 software package (Wyatt Technology) (FIG. 5).

For RSV180913 (SEQ ID NO: 14), RSV190414 (SEQ ID NO: 16), RSV190420 (SEQ ID NO: 17) and RSV200125 (SEQ ID NO: 18) the analysis was performed using a Ultra High-Performance Liquid Chromatography (UHPLC) using a Vanquish system (ThermoFisher Scientific) with a Sepax Unix-C SEC-300 4.6×150 mm 1.8 μm column (Sepax (231300-4615), injection volume 20 μL, flow 0.3 mL/min.). The elution was monitored by a UV detector (Thermo Fisher Scientific), a μDawn Light Scatter (LS) detector (Wyatt Technologies), a μT-rEx Refractive Index (RI) detector (Wyatt Technologies) and a Nanostar Dynamic Light Scattering (DLS) detector (Wyatt Technologies). The trimeric protein has a retention time of about 4.5 minutes. The SEC profiles were analyzed by the Astra 7.3.2.19 software package (Wyatt Technology) and the chromatograms were plotted using GraphPad Prism (version 8) (FIG. 5)

### Results and Discussion:

All RSV pre-fusion F_{B} variants showed high trimer content. For RSV181181 (SEQ ID NO :11) and RSV181182 (SEQ ID NO: 12) minor amounts of aggregates were observed (FIG. 5).

### Stability and Trimer Content of RSV Pre Fusion F_{B} Proteins at 37° C. for 35 Days

The trimer content of the RSV F_{B} proteins after storage for 35 days at 37° C. was assessed by analytical SEC to evaluate the be stabilizing contributions of the different mutations.

For RSV180915 (SEQ ID NO: 6), RSV180916 (SEQ ID NO: 8) and RSV181917 (SEQ ID NO: 9) the analysis was performed using a High Performance Liquid Chromatography (HPLC), for details see method section above.

For RSV181180 (SEQ ID NO: 10), RSV181181 (SEQ ID NO: 11), and RSV181182 (SEQ ID NO: 12) the analysis was performed using a Ultra High-Performance Liquid Chromatography (UHPLC) for details see method section above.

### Results and Discussion:

After storage of the purified RSV pre-fusion FB proteins at 37° C. for 35 days, RSV180915 (SEQ ID NO: 6), RSV180916 (SEQ ID NO: 8), RSV180917 (SEQ ID NO: 9) and RSV181182 (SEQ ID NO: 12) variants remained stable as evaluated by analytical SEC (FIG. 6). RSV181180 (SEQ ID NO: 10) and RSV181181 (SEQ ID NO: 11) contained an increased amount of aggregates compared to non-stressed material (FIG. 5) suggesting that the L2031 mutation is important for long term stability. The stabilizing effect of D489Y (RSV181181 (SEQ ID NO: 11)) and T357R and N371Y (RSV181182 (SEQ ID NO: 12)) was shown by the reduced amount of aggregation compared to RSV181180 (SEQ ID NO: 10).

### Temperature Stability of the RSV F Polypeptides Based on Melting Temperature

The melting temperatures of the purified polypeptides RSV180915 (SEQ ID NO: 6), RSV180916 (SEQ ID NO: 8), RSV180917 (SEQ ID NO: 9), RSV181180 (SEQ ID NO: 10), RSV181181 (SEQ ID NO: 11), RSV181182 (SEQ ID NO: 12), RSV190913 (SEQ ID NO: 14), RSV190414 (SEQ ID NO: 16), RSV190420 (SEQ ID NO: 17) and RSV200125 (SEQ ID NO: 18) were determined by differential scanning fluorometry (DSF). The purified pre-fusion F protein was mixed with SYPRO orange fluorescent dye (Life Technologies 56650) in a 96-well optical qPCR plate. The optimal dye and protein concentration was determined experimentally (data not shown). Protein dilutions were performed in PBS, and a negative control sample containing the dye only was used as a reference subtraction. The measurement was performed in a qPCR instrument (Applied Biosystems ViiA 7) using the following parameters: a temperature ramp from 25-95° C. with a rate of 0.015° C. per second. Data was collected continuously. The melting curves were plotted using GraphPad PRISM software (version 8) and the Tniso values were calculated by the Spotfire suite (Tibco Software Inc.). Melting temperatures were calculated at the 50% maximum of fluorescence using a non-linear EC50 shift equation.

### Results and Discussion:

The melting temperatures (Tm50) of RSV pre-fusion FB variants with diverse sets of stabilizing mutations ranged from 60 to 71° C. with double or single melting events, see Table 2.

TABLE 2
Temperature stability of purified RSV pre-F type B polypeptides

| | | TM50 in C. ° | |
|---|---|---|---|
| # | Polypeptide design | 1st | 2nd |
| RSV181177 | None stabilized | NA | NA |
| RSV181178 | I152M + K226M | NA | NA |
| RSV181179 | I152M + K226M + D486N | NA | NA |
| RSV180915 | I152M + K226M + D486N + S215P | 60.5 | 65.6 |
| RSV180916 | I152M + K226M + D486N + S215P + L203I + P101Q | — | 66.6 |
| RSV180917 | I152M + K226M + D486N + S215P + L203I + P101Q + D489Y + T357R + N371Y | 64 | 71.9 |
| RSV181180 | I152M + K226M + D486N + S215P + P101Q | ~61 | 65.7 |
| RSV181181 | I152M + K226M + D486N + S215P + P101Q + D489Y | 62 | 69.9 |
| RSV181182 | I152M + K226M + D486N + S215P + P101Q + D489Y + T357R + N371Y | 62.5 | 71.6 |
| RSV180913 | I152M + K226M + D486N + S215P + L203I + P101Q | — | 66.3 |
| RSV190417 | I152M + K226M + D486N + S215P + L203I + P101Q + D489Y | NA | NA |
| RSV190414 | I152M + D486N + S215P + L203I + P101Q + D489Y | — | 70.5 |
| RSV190420 | I152M + D486N + S215P + L203I + P101Q + D489Y + L172Q + S173L | — | 70.6 |
| RSV200125 | I152M + D486N + S215P + L203I + P101Q + D489Y + L172Q + S173L + Q191R + I206M + Q209R | — | 71 |

NA = Not available

Generally, a defined high single melting event is preferred. A single melting event with highest stability was shown for RSV190414 (SEQ ID NO: 16). Addition of drift mutations at position 172, 173 (RSV190420 (SEQ ID NO: 17) and 191, 206 and 209 (RSV200125 (SEQ ID NO: 18)) did not decrease the stability.

### Cryostability of the RSV Pref Type B Trimers

The RSV F_{B} trimers of RSV180913 (SEQ ID NO :14), RSV190420 (SEQ ID NO: 17) and RSV200125 (SEQ ID NO: 18) were dialyzed to formulation buffer 1 or to formulation buffer 2 and each formulation was diluted to 0.3 mg/ml of RSV protein. Formulation buffer 1 and 2 are TRIS-based or Phosphate based, respectively. Of each formulation 0.75 ml was filled in glass injection vials with rubber stopper and sealed with aluminum caps. Vials were slowly frozen to −70° C. in 24 hours. Samples were subsequently thawed to RT and analyzed by analytical Size Exclusion Chromatography (SEC). SEC analysis was performed using an Ultra High-Performance Liquid Chromatography (UHPLC) using a Vanquish system (ThermoFisher Scientific), for method details see previous section trimer content.

### Results and Discussion:

In formulation buffer 1 and 2 the polypeptides stay trimeric after freezing (FIG. 7). RSV190420 and RSV200125 were very stable in both buffers. The addition of stabilizing mutations D489Y as in RSV190420 (SEQ ID NO: 17) and RSV200125 (SEQ ID NO: 18) reduced trimer loss after freezing in formulation buffer 2.

### Example 5: Antigenicity of the Preferred Polypeptides

Binding of antibodies to the polypeptides RSV190913 (SEQ ID NO: 14), RSV190414 (SEQ ID NO: 16), RSV190420 (SEQ ID NO: 17) and RSV200125 (SEQ ID NO: 18) were measured by Enzyme-Linked Immuno Sorbent Assay (ELISA). First, 96-well half area HB plates (Perkin Elmer, cat#6002290) were coated with different antibodies (1 μg/mL) in Phosphate Buffer Saline (PBS), 504/well and the plates were incubated overnight at 4° C. Pre-fusion specific antibodies used: CR9501, ADI-18933, ADI-18882, ADI-18930, ADI-18928, ADI-15594, ADI-18889, ADI-18913, and ADI-15617 (Gilman et al., 2016), RSDS-GL (Jones et al.,2019), and hRSV90 (Mousa et al., 2017). Pre-fusion and post-fusion binding antibody used: CR9506 which comprises a heavy chain variable region comprising SEQ ID NO: 21, and a light chain variable region comprising SEQ ID NO: 22). Post-fusion antibody used: ADI-15644 (Gilman et al. 2016). After incubation overnight, the plates were washed 3 times with 100 μL wash buffer (PBS+0.05% Tween20). To each well 100 μL blocking buffer was added (2% Bovine Serum Albumin (BSA), 0.05% Tween20 in PBS) and the plates were incubated for 1 hour at room temperature, shaking. Next, the plates were washed 3 times with 100 μL wash buffer (PBS+0.05% Tween20). For the sample preparation, the protein samples were first diluted to 4 μg/mL in assay buffer (1% BSA, 0.05% Tween20 in PBS). The 4 μg/mL samples were diluted further 4-fold by adding 250 μL dilution to 750 μLassay buffer. The plates were incubated for 1 hour at room temperature, shaking. After incubation the plates were washed 3 times with 300 μL wash buffer. To each well 50 μL of the pre- and post-fusion binding antibody CR9506 with a horseradish peroxidase (HRP) label was added at a concentration of 0.05 μg/mL. The plates were incubated for 1 hour at room temperature, shaking. After incubation the plates were washed 3 times with 300 μL wash buffer and to each well 20 μL of the POD substrate was added. The plates were measured (EnSight Multimode Plate reader, HH34000000, reading Luminescence) between 5 -15 minutes after addition of the substrate. The ELISA curve (protein dilution vs. RLU) were plotted in GraphPad Prism and GraphPad Prism was used to calculate the IC50 values (Table 3).

TABLE 3
IC50 values of RSV F variants

| IC50s (log | | Antigenic | RSV150042 | | RSV150043 | | RSV180913 | | RSV190414 | RSV190420 | | RSV200125 |
|---|---|---|---|---|---|---|---|---|---|---|---|---|
| ug/ml) | Specificity | site | n = 2 | SD | n = 2 | SD | n = 2 | SD | n = 1 | n = 2 | SD | n = 1 |
| CR9501 | preF | V/Ø | 0.139 | 0.050 | NB | NB | 0.221 | 0.135 | 0.179 | 0.160 | 0.042 | 0.124 |
| CR9506 | preF + postF | II | 0.077 | 0.025 | 0.092 | 0.043 | 0.126 | 0.094 | 0.148 | 0.074 | 0.009 | 0.064 |
| ADI-15644 | postF | NA | NB | NA | 0.026 | 0.010 | NB | NA | NB | NB | NA | NB |
| ADI-18933 | preF | Ø** | 0.156 | 0.048 | NB | NB | 0.243 | 0.169 | 0.259 | NB | NA | NB |
| ADI-18882 | | | 0.106 | NA | NB | NB | 0.082 | NA | NA | 0.092 | NA | 0.081 |
| ADI-18930 | | | 0.114 | 0.046 | NB | NB | 0.112 | 0.068 | 0.086 | 0.082 | 0.019 | 0.063 |
| ADI-18928 | | | 0.178 | 0.035 | NB | NB | 0.210 | 0.170 | 0.138 | 0.152 | 0.066 | 0.107 |
| ADI-15594 | | | 0.048 | 0.022 | NB | NB | 0.050 | 0.023 | 0.042 | 0.042 | 0.000 | 0.048 |
| ADI-18889 | | | 0.114 | 0.035 | NB | NB | 0.123 | 0.080 | 0.091 | 0.087 | 0.019 | 0.073 |
| ADI-18913 | | | 0.033 | 0.007 | NB | NB | 0.241 | 0.167 | 0.119 | 0.170 | 0.064 | 0.347 |
| ADI-15617 | | | 0.142 | 0.060 | NB | NB | 0.166 | 0.097 | 0.164 | 0.115 | 0.002 | 0.106 |
| RSD5-GL | | | 0.024 | 0.005 | NB | NB | 0.034 | 0.017 | 0.029 | 0.025 | 0.003 | 0.022 |
| hRSV90 | preF | V | 0.151 | 0.095 | NB | NB | 0.201 | 0.121 | 0.175 | NB | NA | NB |

n—number of experiments;
SD—standard deviation;
NA—not available;
NB—not binding;
**in literature described site zero binders

### Results and Discussion:

None of the purified RSV pre-fusion FB trimers showed binding to post-fusion specific monoclonal antibody ADI-15644, whereas the post-fusion protein RSV150043 (SEQ ID NO: 20) did bind. The IC50 values of RSV prefusion F proteins RSV190913 (SEQ ID NO: 14), RSV190414 (SEQ ID NO: 16), RSV190420 (SEQ ID NO: 17), RSV200125 (SEQ ID NO: 18) and RSV150042 (SEQ ID NO: 19) were comparable for CR9501, CR9506, ADI-18882, ADI-18930, ADI-18928, ADI-15594, ADI-18889, ADI-15617 and RSDS-GL

For pre-fusion specific antibodies ADI-18933 and hRSV90 no binding was observed with RSV190420 (SEQ ID NO: 17) and RSV200125 (SEQ ID NO: 18) which can be explained by the differences of those proteins in the antibody footprints compared to RSV180913 (SEQ ID NO: 14) and RSV190414 (SEQ ID NO: 16). Furthermore, no binding was observed for RSV200125 (SEQ ID NO: 18) to pre-fusion specific monoclonal antibody ADI-18913, which can be explained by the differences of this protein in the surface of the antibody footprints compared to RSV180913 (SEQ ID NO: 14), RSV190414 (SEQ ID NO: 16) and RSV190240 (SEQ ID NO: 17).

### Example 6: Expression and Stability of Full-Length, Membrane-Bound RSV B F Variants After Transient Transfection in expiHEK293F Cells

Full-length RSV-FB non-stabilized proteins in either a processed (SEQ ID NO: 25) or a single-chain (SEQ ID NO: 26) form were used as control for expression and stability. These sequences were based on a consensus sequence for subgroup B (SEQ ID NO: 1) that contains an N-terminal signal peptide based on RSV F type A (SEQ ID NO: 23). The C-terminal lysine residue of SEQ ID NO: 1 was changed to asparagine, the corresponding residue in RSV F type A proteins, to prevent C-terminal lysine clipping. Stabilized full-length variants of these RSV F type B polypeptides contained 6 stabilizing amino acid substitutions (i.e. P101Q, I152M, L2031, S215P, D486N and D489Y); (SEQ ID NO: 29) for the processed variant and 5 stabilizing amino acid substitutions (i.e. P101Q, I152M, L2031, S215P, and D486N) (SEQ ID NO: 30) for the single-chain variant, respectively.

DNA fragments encoding the full length RSV FB proteins (SEQ ID NO: 25, 26, 29, 30, 32 and 34) were synthesized (Genscript) and cloned in the pcDNA2004 expression vector (modified pcDNA3 plasmid with an enhanced CMV promotor). The expression platform used was the expi293Freestyle cells (Life Technologies) in 100 ml shaker flasks. The cells were transiently transfected using Expifectamine (Life Technologies) according to the manufacturer's instructions and cultured for 2 days at 37° C. and 10% CO2. Cells were harvested by centrifugation for 5 minutes at 300 g and resuspended in PBS. To measure stability of the preF proteins, cells were subjected to 10 min heat stress at either 37° C. or 55° C.

Fluorescence-activated cell sorting (FACS) was used for measuring pre-fusion RSV F_{B} protein expression on the plasma membrane after heat stress. CR9501 was fluorescently labeled with Alexa488 by standard protocols. Cells were stained for 30 min at 2.5ug/ml of CR9501, washed and analyzed on a FACS Canto II. Data analysis was done using the FlowJo version 10.6.2 software.

### Results and Discussion:

Both processed and single-chain wildtype RSV-F_{B} (PR_wildtype and SC_wildtype, respectively) showed expression of pre-fusion F_{B} protein on the cell surface after incubation at 37° C. as determined by binding with Mab CR9501 (FIG. 8). Binding of Mab CR9501 to pre-fusion F was reduced after incubation at 55° C., indicating that both versions of the wildtype RSV-F_{B} proteins were unstable (FIG. 8). Both processed and single-chain RSV-B F protein containing the stabilizing mutations of this invention (PR stabilized (SEQ ID NO: 32) and SC stabilized (SEQ ID NO: 34), respectively) showed increased expression of pre-fusion F protein on the cell surface after incubation at 37° C. as compared to the wildtype proteins. Moreover, binding was not reduced after incubation at 55° C., confirming that both versions of the stabilized RSV F_{B} proteins indeed had an increased stability.

### Example 7: preF B (RSV190420, SEQ ID NO: 17) is Immunogenic in Mice and Cotton Rats and Dose-Dependently Induces Antibodies Capable of Neutralizing RSV A and RSV B Strains

Balb/c mice were intramuscularly immunized with 15, 5 or 1.5 ug unadjuvanted RSV190420 protein at day 0 and 28 (n=6 per group). Cotton rats were intramuscularly immunized with 50 or 5 ug unadjuvanted RSV190420, or with 5 ug RSV190420 adjuvanted with AdjuPhos at day 0 and day 28 (n=10 per group). Control groups received intramuscular immunization with formulation buffer (F_{B—) (n=}3 for mice, n=7 for cotton rats). Virus neutralizing antibody responses were measured at day 42 for mice, or day 49 for cotton rats.

Responses were measured using a firefly luciferase-based (FFL) assay for strains RSV A CL57 or RSV B1, using a Plaque Reduction Neutralization Test (PRNT) for strains RSV A2 or RSV B Wash, or using a microneutralization (MN) assay for clinical isolates RSV B 11-052099 and RSV B 17-058221.

### Results and Discussion:

Intramuscular immunization of mice or cotton rats with preF-B protein RSV190420 results in a dose-dependent induction of antibodies, which were capable to neutralize different RSV A and RSV B strains, when assayed using various types of virus neutralization assays (FIG. 9). These results demonstrate that preF-B protein is immunogenic in rodents and induces cross-neutralizing antibodies.

### Example 8: preF B (RSV200125, SEQ ID NO: 18) is Immunogenic in Cotton Rats and Induces Protection Against Challenge with RSV A2 or RSV B Wash

Cotton rats were intramuscularly immunized with 50, 5 or 1 ug unadjuvanted RSV200125 at day 0 and day 28 (n=7 per group per challenge virus). A control group received intramuscular immunization with formulation buffer (FB) (n=7). Animals were intranasally challenged at day 49 with RSV A2, or at day 50 with RSV B Wash. Five days post challenge, lung and nose tissue was isolated and viral load was determined in lung and nose homogenates by plaque assay. Pre-challenge sera was isolated at day 49 or day 50, and virus neutralizing antibody responses were measured using a firefly luciferase-based assay (FFL) for strains RSV A CL57 or RSV B1 (day 49 samples only), or using a Plaque Reduction Neutralization Test (PRNT) for strains RSV A2 or RSV B Wash (combined day 49 and day 50 samples).

### Results and Discussion:

Majority of the cotton rats immunized intramuscularly with any of the dose levels of preF-B protein RSV200125 did not have detectable viral load in the lung after challenge with RSV A2 or RSV B Wash. In contrast, in the nose limited protection against RSV A2 was observed, whereas dose-dependent partial protection was observed in the nose against RSV B Wash challenge (FIG. 10A). RSV antibodies were detectable in the pre-challenge serum, capable of neutralizing different RSV A and RSV B strains, when assayed using various FFL-based virus neutralization assay (FIG. 10B) or PRNT (FIG. 10C). These results demonstrate that the preF-B protein is immunogenic and induces protection in RSV A2 and RSV B Wash challenge models in cotton rats.

### Example 9: Adenoviral Vector Encoded Single Chain and Processed RSV preF-B Protein Induces Cellular and Humoral Immune Responses in Mice

Balb/c mice were intramuscularly immunized with 10⁸, 10⁹ or 10¹⁰ viral particles (vp) of Ad26.RSV.preF-B single chain (encoding the stabilized pre-fusion RSV F_{B} protein of SEQ ID NO: 34, or Ad26.RSV.preF-B processed variant (encoding the stabilized RSV F_{B} protein of SEQ ID NO: 32) at day 0 (n=5 per group). A control group received intramuscular immunization with formulation buffer (FB) (n=3). Serum, isolated 6 weeks post immunization, was assayed for virus neutralizing antibody responses using a firefly luciferase-based (FFL) assay for strains RSV A2, RSV A CL57 or RSV B1, and using a Plaque Reduction Neutralization Test (PRNT) for strains RSV A2, or clinical isolate RSV B 18-006171. Splenoctyes isolated at 6 weeks after immunization with the vectors indicated or formulation buffer, were stimulated with peptide pools covering the F sequence from RSV A2, and RSV-F directed IFN-γ responses were measured by ELISPOT.

### Results and Discussion:

Intramuscular immunization of mice with an Adenoviral vector encoding preF-B protein, either in as processed or single chain variant, result in a dose-dependent induction of virus neutralizing antibodies. Whereas relatively low responses towards RSV A strains were observed, clear dose-dependent responses towards various RSV B strains were readily detectable (FIG. 11A and 11B). High RSV F directed cellular responses were induced after single immunization with both vectors (FIG. 11C).

### Example 10: Immunogenicity and Protective Efficacy of preF-B Proteins RSV190414 (SEQ ID NO: 16), RSV190420 (SEQ ID NO: 17) and RSV200125 (SEQ ID NO: 18) in Cotton Rats

RSV preF_{B} proteins were administrated at a 50 μg dose by intramuscular immunization in cotton rats at day 0 and day 28 (n=10 per group). A control group received intramuscular immunization with formulation buffer (FB) (n=7) and the animals were intranasally challenged at day 49 with RSV B 17-058221, a recent clinical isolate RSV B strain. Lung and nose viral load were determined by plaque assay in tissue homogenates isolated 5 days post challenge (see FIG. 12A). Pre-challenge serum samples were analyzed for neutralizing antibodies against the RSV strains indicated by a firefly luciferase-based assay (FIG. 12B), or by microneutralization assay (FIG. 12C). Symbols represent viral load or neutralizing titers of individual animals, whereas mean titers are indicated with horizontal lines. Lower limit of detection or qualification is indicated with a dotted line.

### Results and Discussion:

Cotton rats immunized intramuscularly with any of the different preFn proteins did not have detectable viral load in the lung after challenge with RSV B 17-058221. Whereas full protection in the nose was observed in animals immunized with RSV2000125, several animals with breakthrough nose infection were observed in the groups immunized with RSV190420 or RSV190414 (FIG. 12A). RSV antibodies were detectable in the pre-challenge serum, capable of neutralizing different RSV A and RSV B strains, when assayed using FFL-based virus neutralization assays (FIG. 12B) or microneutralization assays (FIG. 12C). These results demonstrate that the different preF-B proteins are immunogenic and induce protection in RSV B 17-058221 challenge models in cotton rats.

### Example 11: Immunogenicity and Protective Efficacy of Ad26 Encoding Processed preF-B (SEQ ID NO: 32) in Cotton Rats

Ad26.RSV-B.preF was administrated at the dose levels indicated by intramuscular immunization in cotton rats at day 0(n=6 or n=7 per group). Control groups received intramuscular immunization with formulation buffer (n=7). Animals were intranasally challenged at day 49 with RSV A2 or with RSV B 17-058221, a recent clinical isolate RSV B strain. Lung and nose viral load were determined by plaque assay in tissue homogenates isolated 5 days post challenge (See FIG. 13A). Pre-challenge serum samples were analyzed for neutralizing antibodies against the RSV strains indicated by microneutralization assay (FIG. 13B). Symbols represent viral load or neutralizing titers of individual animals, whereas mean titers are indicated with horizontal lines. Lower limit of detection or qualification is indicated with a dotted line.

### Results and Discussion:

Cotton rats immunized intramuscularly with Ad26.RSV-B.preF did not have detectable viral load in the lung after challenge with RSV A2 or RSV B 17-058221, with only few cases of breakthrough lung infection in animals immunized with the lowest Ad26.RSV-B.preF dose. Also, full protection in the nose was observed from RSV B 17-058221 infection at vaccine doses of 10⁷ vp and higher. In contrast, Ad26.RSV-B.preF did not provide full nose protection after RSV A2 challenge, although vaccine dose dependent reduction in nose viral load was observed (FIG. 13A). RSV antibodies were detectable in the pre-challenge serum, capable of neutralizing RSV A and RSV B strains, when assayed using microneutralization assays (FIG. 13B). These results demonstrate that Ad26.RSV-B.preF is immunogenic and induce protection in RSV A2 and RSV B 17-058221 challenge models in cotton rats.

TABLE 1
Standard amino acids, abbreviations and properties

| | 3- | 1- | Side chain | Side chain |
|---|---|---|---|---|
| Amino Acid | Letter | Letter | polarity | charge (pH 7.4) |
| alanine | Ala | A | non-polar | Neutral |
| arginine | Arg | R | polar | Positive |
| asparagine | Asn | N | polar | Neutral |
| aspartic acid | Asp | D | polar | Negative |
| cysteine | Cys | C | non-polar | Neutral |
| glutamic acid | Glu | E | polar | Negative |
| glutamine | Gln | Q | polar | Neutral |
| glycine | Gly | G | non-polar | Neutral |
| histidine | His | H | polar | Positive (10%) |
| | | | | neutral(90%) |
| isoleucine | Ile | I | non-polar | Neutral |
| leucine | Leu | L | non-polar | Neutral |
| lysine | Lys | K | polar | Positive |
| methionine | Met | M | non-polar | Neutral |
| phenylalanine | Phe | F | non-polar | Neutral |
| proline | Pro | P | non-polar | Neutral |
| serine | Ser | S | polar | Neutral |
| threonine | Thr | T | polar | Neutral |
| tryptophan | Trp | W | non-polar | Neutral |
| tyrosine | Tyr | Y | polar | Neutral |
| valine | Val | V | non-polar | Neutral |

Sequences

RSV F B consensus full length (SEQ ID NO: 1)
MELLIHRSSAIFLTLAINALYLTSSQNITEEFYQSTCSAVSRGYLSALRTGWYTSVITIE
LSNIKETKCNGTDTKVKLIKQELDKYKNAVTELQLLMQNTPAANNRARREAPQYM
NYTINTTKNLNVSISKKRKRRFLGFLLGVGSAIASGIAVSKVLHLEGEVNKIKNALLS
TNKAVVSLSNGVSVLTSKVLDLKNYINNQLLPIVNQQSCRISNIETVIEFQQKNSRLLE
ITREFSVNAGVTTPLSTYMLTNSELLSLINDMPITNDQKKLMSSNVQIVRQQSYSIMSI
IKEEVLAYVVQLPIYGVIDTPCWKLHTSPLCTTNIKEGSNICLTRTDRGWYCDNAGSV
SFFPQADTCKVQSNRVFCDTMNSLTLPSEVSLCNTDIFNSKYDCKIMTSKTDISSSVIT
SLGAIVSCYGKTKCTASNKNRGIIKTFSNGCDYVSNKGVDTVSVGNTLYYVNKLEG
KNLYVKGEPIINYYDPLVFPSDEFDASISQVNEKINQSLAFIRRSDELLHNVNTGKSTT
NIMITAIIIVIIVVLLSLIAIGLLLYCKAKNTPVTLSKDQLSGINNIAFSK
SEQ ID NO: 2 (fibritin)
GYIPEAPRDGQAYVRKDGEWVLLSTFL
SEQ ID NO: 3 RSV181177 RSV F B consensus soluble polypeptide with RSV F_{A} signal peptide and
foldon underlined; p27 bold and underlined; linkers in italic; C-tag in bold.
MELLILKANAITTILTAVTFCFASGQNITEEFYQSTCSAVSRGYLSALRTGWYTSVITIELSNIK
ETKCNGTDTKVKLIKQELDKYKNAVTELQLLMQNTPAANNRARREAPQYMNYTINTTKNL
NVSISKKRKRRFLGFLLGVGSAIASGIAVSKVLHLEGEVNKIKNALLSTNKAVVSLSNGVSVL
TSKVLDLKNYINNQLLPIVNQQSCRISNIETVIEFQQKNSRLLEITREFSVNAGVTTPLSTYMLT
NSELLSLINDMPITNDQKKLMSSNVQIVRQQSYSIMSIIKEEVLAYVVQLPIYGVIDTPCWKLH
TSPLCTTNIKEGSNICLTRTDRGWYCDNAGSVSFFPQADTCKVQSNRVFCDTMNSLTLPSEVS
LCNTDIFNSKYDCKIMTSKTDISSSVITSLGAIVSCYGKTKCTASNKNRGIIKTFSNGCDYVSN
KGVDTVSVGNTLYYVNKLEGKNLYVKGEPIINYYDPLVFPSDEFDASISQVNEKINQSLAFIR
RSDELLSAIGGYIPEAPRDGQAYVRKDGEWVLLSTFLGGSEPEA
SEQ ID NO: 4 RSV181178
MELLILKANAITTILTAVTFCFASGQNITEEFYQSTCSAVSRGYLSALRTGWYTSVITIELSNIK
ETKCNGTDTKVKLIKQELDKYKNAVTELQLLMQNTPAANNRARREAPQYMNYTINTTKNL
NVSISKKRKRRFLGFLLGVGSAIASGMAVSKVLHLEGEVNKIKNALLSTNKAVVSLSNGVSV
LTSKVLDLKNYINNQLLPIVNQQSCRISNIETVIEFQQMNSRLLEITREFSVNAGVTTPLSTYML
TNSELLSLINDMPITNDQKKLMSSNVQIVRQQSYSIMSIIKEEVLAYVVQLPIYGVIDTPCWKL
HTSPLCTTNIKEGSNICLTRTDRGWYCDNAGSVSFFPQADTCKVQSNRVFCDTMNSLTLPSEV
SLCNTDIFNSKYDCKIMTSKTDISSSVITSLGAIVSCYGKTKCTASNKNRGIIKTFSNGCDYVSN
KGVDTVSVGNTLYYVNKLEGKNLYVKGEPIINYYDPLVFPSDEFDASISQVNEKINQSLAFIR
RSDELLSAIGGYIPEAPRDGQAYVRKDGEWVLLSTFLGGSEPEA
SEQ ID NO: 5 RSV181179
MELLILKANAITTILTAVTFCFASGQNITEEFYQSTCSAVSRGYLSALRTGWYTSVITIELSNIK
ETKCNGTDTKVKLIKQELDKYKNAVTELQLLMQNTPAANNRARREAPQYMNYTINTTKNL
NVSISKKRKRRFLGFLLGVGSAIASGMAVSKVLHLEGEVNKIKNALLSTNKAVVSLSNGVSV
LTSKVLDLKNYINNQLLPIVNQQSCRISNIETVIEFQQMNSRLLEITREFSVNAGVTTPLSTYML
TNSELLSLINDMPITNDQKKLMSSNVQIVRQQSYSIMSIIKEEVLAYVVQLPIYGVIDTPCWKL
HTSPLCTTNIKEGSNICLTRTDRGWYCDNAGSVSFFPQADTCKVQSNRVFCDTMNSLTLPSEV
SLCNTDIFNSKYDCKIMTSKTDISSSVITSLGAIVSCYGKTKCTASNKNRGIIKTFSNGCDYVSN
KGVDTVSVGNTLYYVNKLEGKNLYVKGEPIINYYDPLVFPSNEFDASISQVNEKINQSLAFIR
RSDELLSAIGGYIPEAPRDGQAYVRKDGEWVLLSTFLGGSEPEA
SEQ ID NO: 6 RSV180915
MELLILKANAITTILTAVTFCFASGQNITEEFYQSTCSAVSRGYLSALRTGWYTSVITIELSNIK
ETKCNGTDTKVKLIKQELDKYKNAVTELQLLMQNTPAANNRARREAPQYMNYTINTTKNL
NVSISKKRKRRFLGFLLGVGSAIASGMAVSKVLHLEGEVNKIKNALLSTNKAVVSLSNGVSV
LTSKVLDLKNYINNQLLPIVNQQSCRIPNIETVIEFQQMNSRLLEITREFSVNAGVTTPLSTYML
TNSELLSLINDMPITNDQKKLMSSNVQIVRQQSYSIMSIIKEEVLAYVVQLPIYGVIDTPCWKL
HTSPLCTTNIKEGSNICLTRTDRGWYCDNAGSVSFFPQADTCKVQSNRVFCDTMNSLTLPSEV
SLCNTDIFNSKYDCKIMTSKTDISSSVITSLGAIVSCYGKTKCTASNKNRGIIKTFSNGCDYVSN
KGVDTVSVGNTLYYVNKLEGKNLYVKGEPIINYYDPLVFPSNEFDASISQVNEKINQSLAFIR
RSDELLSAIGGYIPEAPRDGQAYVRKDGEWVLLSTFLGGSEPEA
SEQ ID NO: 7 RSV180910 (RSV F_{B} signal underlined)
MELLIHRSSAIFLTLAINALYLTSSQNITEEFYQSTCSAVSRGYLSALRTGWYTSVITIELSNIKE
TKCNGTDTKVKLIKQELDKYKNAVTELQLLMQNTQAANNRARREAPQYMNYTINTTKNLN
VSISKKRKRRFLGFLLGVGSAIASGMAVSKVLHLEGEVNKIKNALLSTNKAVVSLSNGVSVL
TSKVLDLKNYINNQILPIVNQQSCRIPNIETVIEFQQMNSRLLEITREFSVNAGVTTPLSTYMLT
NSELLSLINDMPITNDQKKLMSSNVQIVRQQSYSIMSIIKEEVLAYVVQLPIYGVIDTPCWKLH
TSPLCTTNIKEGSNICLTRTDRGWYCDNAGSVSFFPQADTCKVQSNRVFCDTMNSLTLPSEVS
LCNTDIFNSKYDCKIMTSKTDISSSVITSLGAIVSCYGKTKCTASNKNRGIIKTFSNGCDYVSN
KGVDTVSVGNTLYYVNKLEGKNLYVKGEPIINYYDPLVFPSNEFDASISQVNEKINQSLAFIR
RSDELLSAIGGYIPEAPRDGQAYVRKDGEWVLLSTFLGGSEPEA
SEQ ID NO: 8 RSV180916
MELLILKANAITTILTAVTFCFASGQNITEEFYQSTCSAVSRGYLSALRTGWYTSVITIELSNIK
ETKCNGTDTKVKLIKQELDKYKNAVTELQLLMQNTQAANNRARREAPQYMNYTINTTKNL
NVSISKKRKRRFLGFLLGVGSAIASGMAVSKVLHLEGEVNKIKNALLSTNKAVVSLSNGVSV
LTSKVLDLKNYINNQILPIVNQQSCRIPNIETVIEFQQMNSRLLEITREFSVNAGVTTPLSTYML
TNSELLSLINDMPITNDQKKLMSSNVQIVRQQSYSIMSIIKEEVLAYVVQLPIYGVIDTPCWKL
HTSPLCTTNIKEGSNICLTRTDRGWYCDNAGSVSFFPQADTCKVQSNRVFCDTMNSLTLPSEV
SLCNTDIFNSKYDCKIMTSKTDISSSVITSLGAIVSCYGKTKCTASNKNRGIIKTFSNGCDYVSN
KGVDTVSVGNTLYYVNKLEGKNLYVKGEPIINYYDPLVFPSNEFDASISQVNEKINQSLAFIR
RSDELLSAIGGYIPEAPRDGQAYVRKDGEWVLLSTFLGGSEPEA
SEQ ID NO: 9 RSV180917
MELLILKANAITTILTAVTFCFASGQNITEEFYQSTCSAVSRGYLSALRTGWYTSVITIELSNIK
ETKCNGTDTKVKLIKQELDKYKNAVTELQLLMQNTQAANNRARREAPQYMNYTINTTKNL
NVSISKKRKRRFLGFLLGVGSAIASGMAVSKVLHLEGEVNKIKNALLSTNKAVVSLSNGVSV
LTSKVLDLKNYINNQILPIVNQQSCRIPNIETVIEFQQMNSRLLEITREFSVNAGVTTPLSTYML
TNSELLSLINDMPITNDQKKLMSSNVQIVRQQSYSIMSIIKEEVLAYVVQLPIYGVIDTPCWKL
HTSPLCTTNIKEGSNICLTRTDRGWYCDNAGSVSFFPQADRCKVQSNRVFCDTMYSLTLPSEV
SLCNTDIFNSKYDCKIMTSKTDISSSVITSLGAIVSCYGKTKCTASNKNRGIIKTFSNGCDYVSN
KGVDTVSVGNTLYYVNKLEGKNLYVKGEPIINYYDPLVFPSNEFYASISQVNEKINQSLAFIR
RSDELLSAIGGYIPEAPRDGQAYVRKDGEWVLLSTFLGGSEPEA
SEQ ID NO: 10 RSV181180
MELLILKANAITTILTAVTFCFASGQNITEEFYQSTCSAVSRGYLSALRTGWYTSVITIELSNIK
ETKCNGTDTKVKLIKQELDKYKNAVTELQLLMQNTQAANNRARREAPQYMNYTINTTKNL
NVSISKKRKRRFLGFLLGVGSAIASGMAVSKVLHLEGEVNKIKNALLSTNKAVVSLSNGVSV
LTSKVLDLKNYINNQLLPIVNQQSCRIPNIETVIEFQQMNSRLLEITREFSVNAGVTTPLSTYML
TNSELLSLINDMPITNDQKKLMSSNVQIVRQQSYSIMSIIKEEVLAYVVQLPIYGVIDTPCWKL
HTSPLCTTNIKEGSNICLTRTDRGWYCDNAGSVSFFPQADTCKVQSNRVFCDTMNSLTLPSEV
SLCNTDIFNSKYDCKIMTSKTDISSSVITSLGAIVSCYGKTKCTASNKNRGIIKTFSNGCDYVSN
KGVDTVSVGNTLYYVNKLEGKNLYVKGEPIINYYDPLVFPSNEFDASISQVNEKINQSLAFIR
RSDELLSAIGGYIPEAPRDGQAYVRKDGEWVLLSTFLGGSEPEA
SEQ ID NO: 11 RSV181181
MELLILKANAITTILTAVTFCFASGQNITEEFYQSTCSAVSRGYLSALRTGWYTSVITIELSNIK
ETKCNGTDTKVKLIKQELDKYKNAVTELQLLMQNTQAANNRARREAPQYMNYTINTTKNL
NVSISKKRKRRFLGFLLGVGSAIASGMAVSKVLHLEGEVNKIKNALLSTNKAVVSLSNGVSV
LTSKVLDLKNYINNQLLPIVNQQSCRIPNIETVIEFQQMNSRLLEITREFSVNAGVTTPLSTYML
TNSELLSLINDMPITNDQKKLMSSNVQIVRQQSYSIMSIIKEEVLAYVVQLPIYGVIDTPCWKL
HTSPLCTTNIKEGSNICLTRTDRGWYCDNAGSVSFFPQADTCKVQSNRVFCDTMNSLTLPSEV
SLCNTDIFNSKYDCKIMTSKTDISSSVITSLGAIVSCYGKTKCTASNKNRGIIKTFSNGCDYVSN
KGVDTVSVGNTLYYVNKLEGKNLYVKGEPIINYYDPLVFPSNEFYASISQVNEKINQSLAFIR
RSDELLSAIGGYIPEAPRDGQAYVRKDGEWVLLSTFLGGSEPEA
SEQ ID NO: 12 RSV181182
MELLILKANAITTILTAVTFCFASGQNITEEFYQSTCSAVSRGYLSALRTGWYTSVITIELSNIK
ETKCNGTDTKVKLIKQELDKYKNAVTELQLLMQNTQAANNRARREAPQYMNYTINTTKNL
NVSISKKRKRRFLGFLLGVGSAIASGMAVSKVLHLEGEVNKIKNALLSTNKAVVSLSNGVSV
LTSKVLDLKNYINNQLLPIVNQQSCRIPNIETVIEFQQMNSRLLEITREFSVNAGVTTPLSTYML
TNSELLSLINDMPITNDQKKLMSSNVQIVRQQSYSIMSIIKEEVLAYVVQLPIYGVIDTPCWKL
HTSPLCTTNIKEGSNICLTRTDRGWYCDNAGSVSFFPQADRCKVQSNRVFCDTMYSLTLPSEV
SLCNTDIFNSKYDCKIMTSKTDISSSVITSLGAIVSCYGKTKCTASNKNRGIIKTFSNGCDYVSN
KGVDTVSVGNTLYYVNKLEGKNLYVKGEPIINYYDPLVFPSNEFYASISQVNEKINQSLAFIR
RSDELLSAIGGYIPEAPRDGQAYVRKDGEWVLLSTFLGGSEPEA
SEQ ID NO: 13 RSV180907 (tag-free)
MELLIHRSSAIFLTLAINALYLTSSQNITEEFYQSTCSAVSRGYLSALRTGWYTSVITIELSNIKE
TKCNGTDTKVKLIKQELDKYKNAVTELQLLMQNTQAANNRARREAPQYMNYTINTTKNLN
VSISKKRKRRFLGFLLGVGSAIASGMAVSKVLHLEGEVNKIKNALLSTNKAVVSLSNGVSVL
TSKVLDLKNYINNQILPIVNQQSCRIPNIETVIEFQQMNSRLLEITREFSVNAGVTTPLSTYMLT
NSELLSLINDMPITNDQKKLMSSNVQIVRQQSYSIMSIIKEEVLAYVVQLPIYGVIDTPCWKLH
TSPLCTTNIKEGSNICLTRTDRGWYCDNAGSVSFFPQADTCKVQSNRVFCDTMNSLTLPSEVS
LCNTDIFNSKYDCKIMTSKTDISSSVITSLGAIVSCYGKTKCTASNKNRGIIKTFSNGCDYVSN
KGVDTVSVGNTLYYVNKLEGKNLYVKGEPIINYYDPLVFPSNEFDASISQVNEKINQSLAFIR
RSDELLSAIGGYIPEAPRDGQAYVRKDGEWVLLSTFL
SEQ ID NO: 14 RSV180913 (tag-free)
MELLILKANAITTILTAVTFCFASGQNITEEFYQSTCSAVSRGYLSALRTGWYTSVITIELSNIK
ETKCNGTDTKVKLIKQELDKYKNAVTELQLLMQNTQAANNRARREAPQYMNYTINTTKNL
NVSISKKRKRRFLGFLLGVGSAIASGMAVSKVLHLEGEVNKIKNALLSTNKAVVSLSNGVSV
LTSKVLDLKNYINNQILPIVNQQSCRIPNIETVIEFQQMNSRLLEITREFSVNAGVTTPLSTYML
TNSELLSLINDMPITNDQKKLMSSNVQIVRQQSYSIMSIIKEEVLAYVVQLPIYGVIDTPCWKL
HTSPLCTTNIKEGSNICLTRTDRGWYCDNAGSVSFFPQADTCKVQSNRVFCDTMNSLTLPSEV
SLCNTDIFNSKYDCKIMTSKTDISSSVITSLGAIVSCYGKTKCTASNKNRGIIKTFSNGCDYVSN
KGVDTVSVGNTLYYVNKLEGKNLYVKGEPIINYYDPLVFPSNEFDASISQVNEKINQSLAFIR
RSDELLSAIGGYIPEAPRDGQAYVRKDGEWVLLSTFL
SEQ ID NO: 15 RSV190417 (tag-free)
MELLILKANAITTILTAVTFCFASGQNITEEFYQSTCSAVSRGYLSALRTGWYTSVITIELSNIK
ETKCNGTDTKVKLIKQELDKYKNAVTELQLLMQNTQAANNRARREAPQYMNYTINTTKNL
NVSISKKRKRRFLGFLLGVGSAIASGMAVSKVLHLEGEVNKIKNALLSTNKAVVSLSNGVSV
LTSKVLDLKNYINNQILPIVNQQSCRIPNIETVIEFQQMNSRLLEITREFSVNAGVTTPLSTYML
TNSELLSLINDMPITNDQKKLMSSNVQIVRQQSYSIMSIIKEEVLAYVVQLPIYGVIDTPCWKL
HTSPLCTTNIKEGSNICLTRTDRGWYCDNAGSVSFFPQADTCKVQSNRVFCDTMNSLTLPSEV
SLCNTDIFNSKYDCKIMTSKTDISSSVITSLGAIVSCYGKTKCTASNKNRGIIKTFSNGCDYVSN
KGVDTVSVGNTLYYVNKLEGKNLYVKGEPIINYYDPLVFPSNEFYASISQVNEKINQSLAFIR
RSDELLSAIGGYIPEAPRDGQAYVRKDGEWVLLSTFL
SEQ ID NO: 16 RSV190414 (tag-free)
MELLILKANAITTILTAVTFCFASGQNITEEFYQSTCSAVSRGYLSALRTGWYTSVITIELSNIK
ETKCNGTDTKVKLIKQELDKYKNAVTELQLLMQNTQAANNRARREAPQYMNYTINTTKNL
NVSISKKRKRRFLGFLLGVGSAIASGMAVSKVLHLEGEVNKIKNALLSTNKAVVSLSNGVSV
LTSKVLDLKNYINNQILPIVNQQSCRIPNIETVIEFQQKNSRLLEITREFSVNAGVTTPLSTYML
TNSELLSLINDMPITNDQKKLMSSNVQIVRQQSYSIMSIIKEEVLAYVVQLPIYGVIDTPCWKL
HTSPLCTTNIKEGSNICLTRTDRGWYCDNAGSVSFFPQADTCKVQSNRVFCDTMNSLTLPSEV
SLCNTDIFNSKYDCKIMTSKTDISSSVITSLGAIVSCYGKTKCTASNKNRGIIKTFSNGCDYVSN
KGVDTVSVGNTLYYVNKLEGKNLYVKGEPIINYYDPLVFPSNEFYASISQVNEKINQSLAFIR
RSDELLSAIGGYIPEAPRDGQAYVRKDGEWVLLSTFL
SEQ ID NO: 17 RSV190420 (tag-free)
MELLILKANAITTILTAVTFCFASGQNITEEFYQSTCSAVSRGYLSALRTGWYTSVITIELSNIK
ETKCNGTDTKVKLIKQELDKYKNAVTELQLLMQNTQAANNRARREAPQYMNYTINTTKNL
NVSISKKRKRRFLGFLLGVGSAIASGMAVSKVLHLEGEVNKIKNALQLTNKAVVSLSNGVSV
LTSKVLDLKNYINNQILPIVNQQSCRIPNIETVIEFQQKNSRLLEITREFSVNAGVTTPLSTYML
TNSELLSLINDMPITNDQKKLMSSNVQIVRQQSYSIMSIIKEEVLAYVVQLPIYGVIDTPCWKL
HTSPLCTTNIKEGSNICLTRTDRGWYCDNAGSVSFFPQADTCKVQSNRVFCDTMNSLTLPSEV
SLCNTDIFNSKYDCKIMTSKTDISSSVITSLGAIVSCYGKTKCTASNKNRGIIKTFSNGCDYVSN
KGVDTVSVGNTLYYVNKLEGKNLYVKGEPIINYYDPLVFPSNEFYASISQVNEKINQSLAFIR
RSDELLSAIGGYIPEAPRDGQAYVRKDGEWVLLSTFL
SEQ ID NO: 18 RSV200125 (tag-free)
MELLILKANAITTILTAVTFCFASGQNITEEFYQSTCSAVSRGYLSALRTGWYTSVITIELSNIK
ETKCNGTDTKVKLIKQELDKYKNAVTELQLLMQNTQAANNRARREAPQYMNYTINTTKNL
NVSISKKRKRRFLGFLLGVGSAIASGMAVSKVLHLEGEVNKIKNALQLTNKAVVSLSNGVSV
LTSRVLDLKNYINNQILPMVNRQSCRIPNIETVIEFQQKNSRLLEITREFSVNAGVTTPLSTYML
TNSELLSLINDMPITNDQKKLMSSNVQIVRQQSYSIMSIIKEEVLAYVVQLPIYGVIDTPCWKL
HTSPLCTTNIKEGSNICLTRTDRGWYCDNAGSVSFFPQADTCKVQSNRVFCDTMNSLTLPSEV
SLCNTDIFNSKYDCKIMTSKTDISSSVITSLGAIVSCYGKTKCTASNKNRGIIKTFSNGCDYVSN
KGVDTVSVGNTLYYVNKLEGKNLYVKGEPIINYYDPLVFPSNEFYASISQVNEKINQSLAFIR
RSDELLSAIGGYIPEAPRDGQAYVRKDGEWVLLSTFL
SEQ ID NO: 19 RSV150042 (PRPM)
MELLILKANAITTILTAVTFCFASGQNITEEFYQSTCSAVSKGYLSALRTGWYTSVITIELSNIK
EIKCNGTDAKVKLIKQELDKYKNAVTELQLLMQSTPATNNRARRELPRFMNYTLNNAKKTN
VTLSKKRKRRFLGFLLGVGSAIASGVAVSKVLHLEGEVNKIKSALLSTNKAVVSLSNGVSVL
TSKVLDLKNYIDKQLLPIVNKQSCSIPNIETVIEFQQKNNRLLEITREFSVNAGVTTPVSTYMLT
NSELLSLINDMPITNDQKKLMSNNVQIVRQQSYSIMSIIKEEVLAYVVQLPLYGVIDTPCWKL
HTSPLCTTNTKEGSNICLTRTDRGWYCDNAGSVSFFPQAETCKVQSNRVFCDTMNSLTLPSE
VNLCNVDIFNPKYDCKIMTSKTDVSSSVITSLGAIVSCYGKTKCTASNKNRGIIKTFSNGCDYV
SNKGVDTVSVGNTLYYVNKQEGKSLYVKGEPIINFYDPLVFPSNEFDASISQVNEKINQSLAFI
RKSDELLSAIGGYIPEAPRDGQAYVRKDGEWVLLSTFL
SEQ ID NO: 20 RSV150043 (post-fusion)
MELLILKANAITTILTAVTFCFASGQNITEEFYQSTCSAVSKGYLSALRTGWYTSVITIELSNIK
ENKCNGTDAKVKLIKQELDKYKNAVTELQLLMQSTPATNNRARRELPRFMNYTLNNAKKT
NVTLSKKRKRRAIASGVAVSKVLHLEGEVNKIKSALLSTNKAVVSLSNGVSVLTSKVLDLKN
YIDKQLLPIVNKQSCSISNIETVIEFQQKNNRLLEITREFSVNAGVTTPVSTYMLTNSELLSLIND
MPITNDQKKLMSNNVQIVRQQSYSIMSIIKEEVLAYVVQLPLYGVIDTPCWKLHTSPLCTTNT
KEGSNICLTRTDRGWYCDNAGSVSFFPQAETCKVQSNRVFCDTMNSLTLPSEVNLCNVDIFN
PKYDCKIMTSKTDVSSSVITSLGAIVSCYGKTKCTASNKNRGIIKTFSNGCDYVSNKGVDTVS
VGNTLYYVNKQEGKSLYVKGEPIINFYDPLVFPSDEFDASISQVNEKINQSLAFIRKSDELL
SEQ ID NO: 21 CR9506 heavy chain
EVQLVQSGAEVKKPGSSVKVSCKASGGTFSRSLITWVRQAPGQGLEWMGEISLVFGSAKNA
QKFQGRVTITADESTSTAHMEMISLKHEDTAVYYCAAHQYGSGTHNNFWDESELRFDLWGQ
GTLVTVSSASTKGPSVFPLAPSSKSTSGGTAALGCLVKDYFPEPVTVSWNSGALTSGVHTFPA
VLQSSGLYSLSSVVTVPSSSLGTQTYICNVNHKPSNTKVDKRVEPKSCDKTHTCPPCPAPELL
GGPSVFLFPPKPKDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPREPQVYTLPPSREEM
TKNQVSLTCLVKGFYPSDIAVEWESNGQPENNYKTTPPVLDSDGSFFLYSKLTVDKSRWQQG
NVFSCSVMHEALHNHYTQKSLSLSPGK
SEQ ID NO: 22 CR9506 light chain
DIVMTQSPSSLSASVGDRVTIACRASQSIGTYLNWYQQKPGKAPKLLIYAASSLQSGVPSRFS
GSGSGTHFTLAISSLQAEDFATYSCQQSYTIPYTFGQGTKLEIKRTVAAPSVFIFPPSDEQLKSG
TASVVCLLNNFYPREAKVQWKVDNALQSGNSQESVTEQDSKDSTYSLSSTLTLSKADYEKH
KVYACEVTHQGLSSPVTKSFNRGEC
SEQ ID NO: 23 RSV A fusion protein signal peptide
MELLILKANAITTILTAVTFCFASG
SEQ ID NO: 24 RSV B fusion protein signal peptide
MELLIHRSSAIFLTLAINALYLTSS
SEQ ID NO: 25 PR_wildtype (full length RSV B fusion protein with RSV-A signal peptide, as
processed variant)
MELLILKANAITTILTAVTFCFASGQNITEEFYQSTCSAVSRGYLSALRTGWYTSVITIELSNIK
ETKCNGTDTKVKLIKQELDKYKNAVTELQLLMQNTPAANNRARREAPQYMNYTINTTKNL
NVSISKKRKRRFLGFLLGVGSAIASGIAVSKVLHLEGEVNKIKNALQLTNKAVVSLSNGVSVL
TSKVLDLKNYINNQLLPIVNQQSCRISNIETVIEFQQKNSRLLEITREFSVNAGVTTPLSTYMLT
NSELLSLINDMPITNDQKKLMSSNVQIVRQQSYSIMSIIKEEVLAYVVQLPIYGVIDTPCWKLH
TSPLCTTNIKEGSNICLTRTDRGWYCDNAGSVSFFPQADTCKVQSNRVFCDTMNSLTLPSEVS
LCNTDIFNSKYDCKIMTSKTDISSSVITSLGAIVSCYGKTKCTASNKNRGIIKTFSNGCDYVSN
KGVDTVSVGNTLYYVNKLEGKNLYVKGEPIINYYDPLVFPSDEFDASISQVNEKINQSLAFIR
RSDELLHNVNTGKSTTNIMITAIIIVIIVVLLSLIAIGLLLYCKAKNTPVTLSKDQLSGINNIAFSN
SEQ ID NO: 26 SC_wildtype (full length RSV B fusion protein with RSV-A signal peptide, as
single chain variant)
MELLILKANAITTILTAVTFCFASGQNITEEFYQSTCSAVSRGYLSALRTGWYTSVITIELSNIK
ETKCNGTDTKVKLIKQELDKYKNAVTELQLLMQNTPAANNQARGSGSGRSLGFLLGVGSAI
ASGIAVSKVLHLEGEVNKIKNALQLTNKAVVSLSNGVSVLTSKVLDLKNYINNQLLPIVNQQ
SCRISNIETVIEFQQKNSRLLEITREFSVNAGVTTPLSTYMLTNSELLSLINDMPITNDQKKLMS
SNVQIVRQQSYSIMSIIKEEVLAYVVQLPIYGVIDTPCWKLHTSPLCTTNIKEGSNICLTRTDRG
WYCDNAGSVSFFPQADTCKVQSNRVFCDTMNSLTLPSEVSLCNTDIFNSKYDCKIMTSKTDIS
SSVITSLGAIVSCYGKTKCTASNKNRGIIKTFSNGCDYVSNKGVDTVSVGNTLYYVNKLEGK
NLYVKGEPIINYYDPLVFPSDEFDASISQVNEKINQSLAFIRRSDELLHNVNTGKSTTNIMITAII
IVIIVVLLSLIAIGLLLYCKAKNTPVTLSKDQLSGINNIAFSN
SEQ ID NO: 29 PR_stabilized (full length RSV B fusion protein with RSV-A signal peptide, as
processed variant)
MELLILKANAITTILTAVTFCFASGQNITEEFYQSTCSAVSRGYLSALRTGWYTSVITIELSNIK
ETKCNGTDTKVKLIKQELDKYKNAVTELQLLMQNTQAANNRARREAPQYMNYTINTTKNL
NVSISKKRKRRFLGFLLGVGSAIASGMAVSKVLHLEGEVNKIKNALQLTNKAVVSLSNGVSV
LTSKVLDLKNYINNQLLPIVNQQSCRIPNIETVIEFQQKNSRLLEITREFSVNAGVTTPLSTYML
TNSELLSLINDMPITNDQKKLMSSNVQIVRQQSYSIMSIIKEEVLAYVVQLPIYGVIDTPCWKL
HTSPLCTTNIKEGSNICLTRTDRGWYCDNAGSVSFFPQADTCKVQSNRVFCDTMNSLTLPSEV
SLCNTDIFNSKYDCKIMTSKTDISSSVITSLGAIVSCYGKTKCTASNKNRGIIKTFSNGCDYVSN
KGVDTVSVGNTLYYVNKLEGKNLYVKGEPIINYYDPLVFPSNEFYASISQVNEKINQSLAFIR
RSDELLHNVNTGKSTTNIMITAIIIVIIVVLLSLIAIGLLLYCKAKNTPVTLSKDQLSGINNIAFSN
SEQ ID NO: 30 SC_stabilized (full length RSV B fusion protein with RSV-A signal peptide, as
single chain variant)
MELLILKANAITTILTAVTFCFASGQNITEEFYQSTCSAVSRGYLSALRTGWYTSVITIELSNIK
ETKCNGTDTKVKLIKQELDKYKNAVTELQLLMQNTQAANNQARGSGSGRSLGFLLGVGSAI
ASGMAVSKVLHLEGEVNKIKNALQLTNKAVVSLSNGVSVLTSKVLDLKNYINNQILPIVNQQ
SCRIPNIETVIEFQQKNSRLLEITREFSVNAGVTTPLSTYMLTNSELLSLINDMPITNDQKKLMS
SNVQIVRQQSYSIMSIIKEEVLAYVVQLPIYGVIDTPCWKLHTSPLCTTNIKEGSNICLTRTDRG
WYCDNAGSVSFFPQADTCKVQSNRVFCDTMNSLTLPSEVSLCNTDIFNSKYDCKIMTSKTDIS
SSVITSLGAIVSCYGKTKCTASNKNRGIIKTFSNGCDYVSNKGVDTVSVGNTLYYVNKLEGK
NLYVKGEPIINYYDPLVFPSNEFDASISQVNEKINQSLAFIRRSDELLHNVNTGKSTTNIMITAII
IVIIVVLLSLIAIGLLLYCKAKNTPVTLSKDQLSGINNIAFSN
SEQ ID NO: 31 Ad26RSV019
ATGGAACTGCTGATCCTGAAGGCCAACGCCATCACCACAATCCTGACCGCCGTGACCTTT
TGCTTCGCCAGCGGCCAGAACATCACCGAGGAATTCTACCAGAGCACCTGTAGCGCCGT
GTCCAGAGGATATCTGTCTGCCCTGAGAACCGGCTGGTACACCAGCGTGATCACCATCGA
GCTGAGCAACATCAAAGAAACAAAGTGCAACGGCACCGACACCAAAGTGAAGCTGATC
AAGCAAGAGCTGGACAAGTACAAGAATGCCGTGACCGAACTGCAGCTGCTGATGCAGAA
TACCCAGGCCGCCAACAACCGGGCCAGAAGAGAAGCCCCTCAGTACATGAACTACACCA
TCAACACCACCAAGAACCTGAACGTGTCCATCAGCAAGAAGCGGAAGCGGAGATTCCTG
GGCTTTCTGCTCGGAGTGGGATCTGCCATTGCCTCTGGAATGGCCGTGTCTAAGGTGCTG
CATCTGGAAGGCGAAGTGAACAAGATCAAGAACGCCCTGCAGCTGACCAACAAGGCCGT
GGTGTCTCTGTCTAATGGCGTGTCCGTGCTGACCAGCAGAGTGCTGGACCTGAAGAACTA
CATCAACAACCAGCTGCTGCCCATGGTCAACCGGCAGAGCTGCAGAATCCCCAACATCG
AGACAGTGATCGAGTTCCAGCAGAAGAACAGCAGGCTGCTGGAAATCACCCGCGAGTTT
TCTGTGAATGCCGGCGTGACAACCCCTCTGAGCACCTACATGCTGACCAATAGCGAGCTG
CTGAGCCTGATCAACGACATGCCCATCACCAACGACCAGAAAAAGCTGATGAGCAGCAA
CGTGCAGATCGTGCGGCAGCAGAGCTACAGCATCATGAGCATTATCAAAGAAGAGGTGC
TGGCCTACGTGGTGCAGCTGCCTATCTACGGCGTGATCGATACCCCTTGCTGGAAGCTGC
ACACAAGCCCACTGTGCACCACCAATATCAAAGAGGGCAGCAACATCTGCCTGACCAGA
ACCGATAGAGGCTGGTACTGCGATAATGCCGGCAGCGTCAGCTTCTTCCCACAAGCCGAT
ACCTGCAAGGTGCAGAGCAACAGAGTGTTCTGCGACACCATGAACAGCCTGACACTGCC
TAGCGAGGTGTCCCTGTGCAACACCGACATCTTCAACTCTAAGTACGACTGCAAGATCAT
GACCTCCAAGACCGACATCAGCTCCTCCGTGATCACATCTCTGGGCGCCATCGTGTCCTG
CTACGGCAAGACAAAGTGTACCGCCAGCAACAAGAACCGGGGCATCATCAAGACCTTCA
GCAACGGCTGCGACTACGTGTCCAACAAAGGCGTGGACACCGTGTCTGTGGGCAACACC
CTGTACTACGTGAACAAGCTGGAAGGCAAGAATCTGTACGTGAAGGGCGAGCCCATCAT
CAACTACTACGACCCTCTGGTGTTCCCCAGCAACGAGTTCTACGCCAGCATCAGCCAAGT
GAACGAGAAGATCAACCAGAGCCTGGCCTTCATCCGCAGATCCGATGAGCTGCTGCACA
ACGTGAACACCGGCAAGAGCACCACAAACATCATGATCACCGCCATCATCATCGTGATC
ATCGTCGTGCTGCTGTCCCTGATCGCCATCGGACTGCTGCTGTACTGCAAGGCCAAGAAC
ACCCCTGTGACACTGAGCAAGGATCAGCTGAGCGGCATCAACAATATCGCCTTCTCCAAC
SEQ ID NO: 32
The amino acid sequence of the transgene (RSV-B preF protein, processed):
MELLILKANAITTILTAVTFCFASGQNITEEFYQSTCSAVSRGYLSALRTGWYTSVITIELSNIK
ETKCNGTDTKVKLIKQELDKYKNAVTELQLLMQNTQAANNRARREAPQYMNYTINTTKNL
NVSISKKRKRRFLGFLLGVGSAIASGMAVSKVLHLEGEVNKIKNALQLTNKAVVSLSNGVSV
LTSRVLDLKNYINNQLLPMVNRQSCRIPNIETVIEFQQKNSRLLEITREFSVNAGVTTPLSTYM
LTNSELLSLINDMPITNDQKKLMSSNVQIVRQQSYSIMSIIKEEVLAYVVQLPIYGVIDTPCWK
LHTSPLCTTNIKEGSNICLTRTDRGWYCDNAGSVSFFPQADTCKVQSNRVFCDTMNSLTLPSE
VSLCNTDIFNSKYDCKIMTSKTDISSSVITSLGAIVSCYGKTKCTASNKNRGIIKTFSNGCDYVS
NKGVDTVSVGNTLYYVNKLEGKNLYVKGEPIINYYDPLVFPSNEFYASISQVNEKINQSLAFI
RRSDELLHNVNTGKSTTNIMITAIIIVIIVVLLSLIAIGLLLYCKAKNTPVTLSKDQLSGINNIAFS
N
SEQ ID NO: 33: Ad26RSV020
ATGGAACTGCTGATCCTGAAGGCCAACGCCATCACCACAATCCTGACCGCCGTGACCTTT
TGCTTCGCCAGCGGCCAGAACATCACCGAGGAATTCTACCAGAGCACCTGTAGCGCCGT
GTCCAGAGGATATCTGTCTGCCCTGAGAACCGGCTGGTACACCAGCGTGATCACCATCGA
GCTGAGCAACATCAAAGAAACAAAGTGCAACGGCACCGACACCAAAGTGAAGCTGATC
AAGCAAGAGCTGGACAAGTACAAGAATGCCGTGACCGAACTGCAGCTGCTGATGCAGAA
TACCCAGGCCGCCAACAATCAGGCCAGAGGCTCTGGATCTGGCAGAAGCCTGGGATTTC
TGCTCGGCGTGGGATCTGCCATTGCCTCTGGAATGGCCGTGTCTAAGGTGCTGCATCTGG
AAGGCGAAGTGAACAAGATCAAGAACGCCCTGCAGCTGACCAACAAGGCCGTGGTGTCT
CTGTCTAATGGCGTGTCCGTGCTGACCAGCAGAGTGCTGGACCTGAAGAACTACATCAAC
AACCAGCTGCTGCCCATGGTCAACCGGCAGAGCTGCAGAATCCCCAACATCGAGACAGT
GATCGAGTTCCAGCAGAAGAACAGCAGGCTGCTGGAAATCACCCGCGAGTTTTCTGTGA
ATGCCGGCGTGACAACCCCTCTGAGCACCTACATGCTGACCAATAGCGAGCTGCTGAGC
CTGATCAACGACATGCCCATCACCAACGACCAGAAAAAGCTGATGAGCAGCAACGTGCA
GATCGTGCGGCAGCAGAGCTACAGCATCATGAGCATTATCAAAGAAGAGGTGCTGGCCT
ACGTGGTGCAGCTGCCTATCTACGGCGTGATCGATACCCCTTGCTGGAAGCTGCACACAA
GCCCACTGTGCACCACCAATATCAAAGAGGGCAGCAACATCTGCCTGACCAGAACCGAT
AGAGGCTGGTACTGCGATAATGCCGGCAGCGTCAGCTTCTTCCCACAAGCCGATACCTGC
AAGGTGCAGAGCAACAGAGTGTTCTGCGACACCATGAACAGCCTGACACTGCCTAGCGA
GGTGTCCCTGTGCAACACCGACATCTTCAACTCTAAGTACGACTGCAAGATCATGACCTC
CAAGACCGACATCAGCTCCTCCGTGATCACATCTCTGGGCGCCATCGTGTCCTGCTACGG
CAAGACAAAGTGTACCGCCAGCAACAAGAACCGGGGCATCATCAAGACCTTCAGCAACG
GCTGCGACTACGTGTCCAACAAAGGCGTGGACACCGTGTCTGTGGGCAACACCCTGTACT
ACGTGAACAAGCTGGAAGGCAAGAACCTGTACGTGAAGGGCGAGCCCATCATCAACTAC
TACGACCCTCTGGTGTTCCCCAGCAACGAGTTCGATGCCAGCATCAGCCAAGTGAACGA
GAAGATCAACCAGAGCCTGGCCTTCATCAGACGCTCCGATGAGCTGCTGCACAACGTGA
ACACCGGCAAGAGCACCACAAACATCATGATCACCGCCATCATCATCGTGATCATCGTC
GTGCTGCTGTCCCTGATCGCCATCGGACTGCTGCTGTACTGCAAGGCCAAGAACACCCCT
GTGACACTGAGCAAGGATCAGCTGAGCGGCATCAACAATATCGCCTTCTCCAAC
SEQ ID NO: 34 protein encoded by Ad26RSV020 (single chain)
MELLILKANAITTILTAVTFCFASGQNITEEFYQSTCSAVSRGYLSALRTGWYTSVITIELSNIK
ETKCNGTDTKVKLIKQELDKYKNAVTELQLLMQNTQAANNQARGSGSGRSLGFLLGVGSAI
ASGMAVSKVLHLEGEVNKIKNALQLTNKAVVSLSNGVSVLTSRVLDLKNYINNQLLPMVNR
QSCRIPNIETVIEFQQKNSRLLEITREFSVNAGVTTPLSTYMLTNSELLSLINDMPITNDQKKLM
SSNVQIVRQQSYSIMSIIKEEVLAYVVQLPIYGVIDTPCWKLHTSPLCTTNIKEGSNICLTRTDR
GWYCDNAGSVSFFPQADTCKVQSNRVFCDTMNSLTLPSEVSLCNTDIFNSKYDCKIMTSKTD
ISSSVITSLGAIVSCYGKTKCTASNKNRGIIKTFSNGCDYVSNKGVDTVSVGNTLYYVNKLEG
KNLYVKGEPIINYYDPLVFPSNEFDASISQVNEKINQSLAFIRRSDELLHNVNTGKSTTNIMITA
IIIVIIVVLLSLIAIGLLLYCKAKNTPVTLSKDQLSGINNIAFSN

### REFERENCES

Gilman et al., Sci Immunol. 1(6): eaaj1879 (2016)
Jones et al., Plos Pathogens: https://doi.org/10.1371/journal.ppat.1007944 (July 15, 2019)
Krarup et al., Nature Comm. 6:8143, (2015)
Kumaria et al., Virology Journal, 8: 372, (2011)
Letarov et al., Biochemistry Moscow 64: 817-823 (1993)
McLellan, et al. Science 342, 592-598 (2013)
McLellan, et al. Nat Struct Mol Biol 17, 248-250 (2010)
McLellan, et al. Science 340, 1113-1117 (2013)
Mousa et al., Nat Microbiol: 2: 16271. doi:10.1038/nmicrobio1.2016.271 (2017)
S-Guthe et al., J. Mol. Biol. 337: 905-915. (2004)
Swanson, et al. (2011) Proc Natl Acad Sci USA. 2011 Jun 7;108(23):9619-24.

1. A stabilized pre-fusion RSV fusion (F) protein, comprising an F1 and an F2 domain, comprising an amino acid sequence of the F1 and F2 domain of an F protein of an RSV B strain, wherein the amino acid residue at position 101 is Q, the amino acid residue at position 152 is M, the amino acid residue at position 215 is P, and the amino acid residue at position 486 is N.

2. The protein according to claim 1, wherein the amino acid residue at position 489 is Y.

3. The protein according to claim 1, wherein the amino acid residue at position 203 is I.

4. The protein according to claim 1, wherein the amino acid residue at position 226 is M.

5. The protein according to claim 1, wherein the amino acid residue at position 101 is Q, the amino acid residue at position 152 is M, the amino acid residue at position 203 is I, the amino acid residue at position 215 is P, the amino acid residue at position 226 is M, and the amino acid residue at position 486 is N.

6. The protein according to claim 3, wherein the amino acid residue at position 101 is Q, the amino acid residue at position 152 is M, the amino acid residue at position 203 is I, the amino acid residue at position 215 is P, the amino acid residue at position 486 is N and the amino acid residue at position 489 is Y.

7. The protein according to claim 1, wherein the amino acid residue at position 172 is Q and the amino acid residue at position 172 is L.

8. The protein according to claim 1, wherein the amino acid residue at position 191 is R, the amino acid residue at position 206 is M and the amino acid residue at position 209 is R

9. The protein according to claim 1, herein the furin cleavage sites have been deleted.

10. The protein according to claim 1, comprising a truncated F1 domain.

11. The protein according to claim wherein the transmembrane and cytoplasmic domain have been deleted, said transmembrane and cytoplasmic domain comprising the amino acids 514 to 574.

12. The protein according to claim 10, wherein a heterologous trimerization domain has been linked to the truncated F1 domain.

13. The protein according to claim 12, wherein the heterologous trimerization domain is a foldon domain comprising the amino acid sequence of SEQ ID NO:2.

14. The protein according to claim 1, comprising an amino acid sequence selected from the group consisting of SEQ ID NO: 14, SEQ ID NO: 16, SEQ ID NO: 17, SEQ ID NO: 18, SEQ ID NO: 29, SEQ ID NO: 30, SEQ ID NO: 32 and SEQ ID NO: 34, or fragments thereof.

15. The protein according to claim 1, wherein the protein does not comprise a signal peptide, p27 peptide or a tag sequence.

16. A nucleic acid molecule encoding a protein according to claim 1.

17. The nucleic acid molecule according to claim 16, wherein the nucleic acid molecule is DNA or RNA.

18. The nucleic acid molecule according to claim 16, encoding a protein comprising an amino acid sequence selected from the group consisting of SEQ ID NO: 14, SEQ ID NO: 16, SEQ ID NO: 17, SEQ ID NO: 18, SEQ ID NO: 29, SEQ ID NO: 30, SEQ ID NO: 32 and SEQ ID NO: 34, or fragments thereof.

19. A vector comprising [[a]]the nucleic acid according to claim 16.

20. The vector according to claim 19, wherein the vector is a human recombinant adenoviral vector.

21. The vector according to claim 20, wherein the adenoviral vector is a replication-incompetent Ad26 adenoviral vector having a deletion of the E1 region and the E3 region.

22. A composition comprising the protein according to claim 1 and a pharmaceutically acceptable carrier.

23. A composition comprising the protein according to claim 1 and a vector comprising a nucleic acid encoding a protein according to claim 1.

24. A vaccine against RSV comprising the composition according to claim 22.

25. A method for vaccinating a subject against RSV, the method comprising administering to the subject the vaccine according to claim 24.

26. A method for preventing infection and/or replication of RSV in a subject, comprising administering to the subject the vaccine according to claim 24.

27. The method according to claim 26, wherein the prevented infection and/or replication of RSV is characterized by the prevention or reduction of reverse transcriptase polymerase chain reaction (RT PCR)-confirmed RSV-mediated lower respiratory tract disease (LRTD).

28. The method according to claim 26, wherein the prevented infection and/or replication of RSV is characterized by an absent or reduced RSV viral load in the nasal track and/or lungs of the subject.

29. The method according to claim 26, wherein the prevented infection and/or replication of RSV is characterized by an absent or reduced RSV clinical symptom in the subject upon exposure to RSV.

30. An isolated host cell comprising the nucleic acid molecule according to claim 16.

31. An isolated host cell comprising a recombinant human adenovirus of serotype 26 comprising the nucleic acid molecule according to claim 16.


SEQUENCE LISTING (34 sequences)

SEQ ID NO: 1 - protein, 574 aa, Artificial Sequence
  RSV F B consensus full length
MELLIHRSSAIFLTLAINALYLTSSQNITEEFYQSTCSAVSRGYLSALRTGWYTSVITIELSNIKETKCNGTDTKVKLIKQELDKYKNAVTELQLLMQNTPAANNRARREAPQYMNYTINTTKNLNVSISKKRKRRFLGFLLGVGSAIASGIAVSKVLHLEGEVNKIKNALLSTNKAVVSLSNGVSVLTSKVLDLKNYINNQLLPIVNQQSCRISNIETVIEFQQKNSRLLEITREFSVNAGVTTPLSTYMLTNSELLSLINDMPITNDQKKLMSSNVQIVRQQSYSIMSIIKEEVLAYVVQLPIYGVIDTPCWKLHTSPLCTTNIKEGSNICLTRTDRGWYCDNAGSVSFFPQADTCKVQSNRVFCDTMNSLTLPSEVSLCNTDIFNSKYDCKIMTSKTDISSSVITSLGAIVSCYGKTKCTASNKNRGIIKTFSNGCDYVSNKGVDTVSVGNTLYYVNKLEGKNLYVKGEPIINYYDPLVFPSDEFDASISQVNEKINQSLAFIRRSDELLHNVNTGKSTTNIMITAIIIVIIVVLLSLIAIGLLLYCKAKNTPVTLSKDQLSGINNIAFSK

SEQ ID NO: 2 - protein, 27 aa, Artificial Sequence
  fibritin
GYIPEAPRDGQAYVRKDGEWVLLSTFL

SEQ ID NO: 3 - protein, 551 aa, Artificial Sequence
  RSV181177
MELLILKANAITTILTAVTFCFASGQNITEEFYQSTCSAVSRGYLSALRTGWYTSVITIELSNIKETKCNGTDTKVKLIKQELDKYKNAVTELQLLMQNTPAANNRARREAPQYMNYTINTTKNLNVSISKKRKRRFLGFLLGVGSAIASGIAVSKVLHLEGEVNKIKNALLSTNKAVVSLSNGVSVLTSKVLDLKNYINNQLLPIVNQQSCRISNIETVIEFQQKNSRLLEITREFSVNAGVTTPLSTYMLTNSELLSLINDMPITNDQKKLMSSNVQIVRQQSYSIMSIIKEEVLAYVVQLPIYGVIDTPCWKLHTSPLCTTNIKEGSNICLTRTDRGWYCDNAGSVSFFPQADTCKVQSNRVFCDTMNSLTLPSEVSLCNTDIFNSKYDCKIMTSKTDISSSVITSLGAIVSCYGKTKCTASNKNRGIIKTFSNGCDYVSNKGVDTVSVGNTLYYVNKLEGKNLYVKGEPIINYYDPLVFPSDEFDASISQVNEKINQSLAFIRRSDELLSAIGGYIPEAPRDGQAYVRKDGEWVLLSTFLGGSEPEA

SEQ ID NO: 4 - protein, 551 aa, Artificial Sequence
  RSV181178
MELLILKANAITTILTAVTFCFASGQNITEEFYQSTCSAVSRGYLSALRTGWYTSVITIELSNIKETKCNGTDTKVKLIKQELDKYKNAVTELQLLMQNTPAANNRARREAPQYMNYTINTTKNLNVSISKKRKRRFLGFLLGVGSAIASGMAVSKVLHLEGEVNKIKNALLSTNKAVVSLSNGVSVLTSKVLDLKNYINNQLLPIVNQQSCRISNIETVIEFQQMNSRLLEITREFSVNAGVTTPLSTYMLTNSELLSLINDMPITNDQKKLMSSNVQIVRQQSYSIMSIIKEEVLAYVVQLPIYGVIDTPCWKLHTSPLCTTNIKEGSNICLTRTDRGWYCDNAGSVSFFPQADTCKVQSNRVFCDTMNSLTLPSEVSLCNTDIFNSKYDCKIMTSKTDISSSVITSLGAIVSCYGKTKCTASNKNRGIIKTFSNGCDYVSNKGVDTVSVGNTLYYVNKLEGKNLYVKGEPIINYYDPLVFPSDEFDASISQVNEKINQSLAFIRRSDELLSAIGGYIPEAPRDGQAYVRKDGEWVLLSTFLGGSEPEA

SEQ ID NO: 5 - protein, 551 aa, Artificial Sequence
  RSV181179
MELLILKANAITTILTAVTFCFASGQNITEEFYQSTCSAVSRGYLSALRTGWYTSVITIELSNIKETKCNGTDTKVKLIKQELDKYKNAVTELQLLMQNTPAANNRARREAPQYMNYTINTTKNLNVSISKKRKRRFLGFLLGVGSAIASGMAVSKVLHLEGEVNKIKNALLSTNKAVVSLSNGVSVLTSKVLDLKNYINNQLLPIVNQQSCRISNIETVIEFQQMNSRLLEITREFSVNAGVTTPLSTYMLTNSELLSLINDMPITNDQKKLMSSNVQIVRQQSYSIMSIIKEEVLAYVVQLPIYGVIDTPCWKLHTSPLCTTNIKEGSNICLTRTDRGWYCDNAGSVSFFPQADTCKVQSNRVFCDTMNSLTLPSEVSLCNTDIFNSKYDCKIMTSKTDISSSVITSLGAIVSCYGKTKCTASNKNRGIIKTFSNGCDYVSNKGVDTVSVGNTLYYVNKLEGKNLYVKGEPIINYYDPLVFPSNEFDASISQVNEKINQSLAFIRRSDELLSAIGGYIPEAPRDGQAYVRKDGEWVLLSTFLGGSEPEA

SEQ ID NO: 6 - protein, 551 aa, Artificial Sequence
  RSV180915
MELLILKANAITTILTAVTFCFASGQNITEEFYQSTCSAVSRGYLSALRTGWYTSVITIELSNIKETKCNGTDTKVKLIKQELDKYKNAVTELQLLMQNTPAANNRARREAPQYMNYTINTTKNLNVSISKKRKRRFLGFLLGVGSAIASGMAVSKVLHLEGEVNKIKNALLSTNKAVVSLSNGVSVLTSKVLDLKNYINNQLLPIVNQQSCRIPNIETVIEFQQMNSRLLEITREFSVNAGVTTPLSTYMLTNSELLSLINDMPITNDQKKLMSSNVQIVRQQSYSIMSIIKEEVLAYVVQLPIYGVIDTPCWKLHTSPLCTTNIKEGSNICLTRTDRGWYCDNAGSVSFFPQADTCKVQSNRVFCDTMNSLTLPSEVSLCNTDIFNSKYDCKIMTSKTDISSSVITSLGAIVSCYGKTKCTASNKNRGIIKTFSNGCDYVSNKGVDTVSVGNTLYYVNKLEGKNLYVKGEPIINYYDPLVFPSNEFDASISQVNEKINQSLAFIRRSDELLSAIGGYIPEAPRDGQAYVRKDGEWVLLSTFLGGSEPEA

SEQ ID NO: 7 - protein, 551 aa, Artificial Sequence
  RSV180910
MELLIHRSSAIFLTLAINALYLTSSQNITEEFYQSTCSAVSRGYLSALRTGWYTSVITIELSNIKETKCNGTDTKVKLIKQELDKYKNAVTELQLLMQNTQAANNRARREAPQYMNYTINTTKNLNVSISKKRKRRFLGFLLGVGSAIASGMAVSKVLHLEGEVNKIKNALLSTNKAVVSLSNGVSVLTSKVLDLKNYINNQILPIVNQQSCRIPNIETVIEFQQMNSRLLEITREFSVNAGVTTPLSTYMLTNSELLSLINDMPITNDQKKLMSSNVQIVRQQSYSIMSIIKEEVLAYVVQLPIYGVIDTPCWKLHTSPLCTTNIKEGSNICLTRTDRGWYCDNAGSVSFFPQADTCKVQSNRVFCDTMNSLTLPSEVSLCNTDIFNSKYDCKIMTSKTDISSSVITSLGAIVSCYGKTKCTASNKNRGIIKTFSNGCDYVSNKGVDTVSVGNTLYYVNKLEGKNLYVKGEPIINYYDPLVFPSNEFDASISQVNEKINQSLAFIRRSDELLSAIGGYIPEAPRDGQAYVRKDGEWVLLSTFLGGSEPEA

SEQ ID NO: 8 - protein, 551 aa, Artificial Sequence
  RSV180916
MELLILKANAITTILTAVTFCFASGQNITEEFYQSTCSAVSRGYLSALRTGWYTSVITIELSNIKETKCNGTDTKVKLIKQELDKYKNAVTELQLLMQNTQAANNRARREAPQYMNYTINTTKNLNVSISKKRKRRFLGFLLGVGSAIASGMAVSKVLHLEGEVNKIKNALLSTNKAVVSLSNGVSVLTSKVLDLKNYINNQILPIVNQQSCRIPNIETVIEFQQMNSRLLEITREFSVNAGVTTPLSTYMLTNSELLSLINDMPITNDQKKLMSSNVQIVRQQSYSIMSIIKEEVLAYVVQLPIYGVIDTPCWKLHTSPLCTTNIKEGSNICLTRTDRGWYCDNAGSVSFFPQADTCKVQSNRVFCDTMNSLTLPSEVSLCNTDIFNSKYDCKIMTSKTDISSSVITSLGAIVSCYGKTKCTASNKNRGIIKTFSNGCDYVSNKGVDTVSVGNTLYYVNKLEGKNLYVKGEPIINYYDPLVFPSNEFDASISQVNEKINQSLAFIRRSDELLSAIGGYIPEAPRDGQAYVRKDGEWVLLSTFLGGSEPEA

SEQ ID NO: 9 - protein, 551 aa, Artificial Sequence
  RSV180917
MELLILKANAITTILTAVTFCFASGQNITEEFYQSTCSAVSRGYLSALRTGWYTSVITIELSNIKETKCNGTDTKVKLIKQELDKYKNAVTELQLLMQNTQAANNRARREAPQYMNYTINTTKNLNVSISKKRKRRFLGFLLGVGSAIASGMAVSKVLHLEGEVNKIKNALLSTNKAVVSLSNGVSVLTSKVLDLKNYINNQILPIVNQQSCRIPNIETVIEFQQMNSRLLEITREFSVNAGVTTPLSTYMLTNSELLSLINDMPITNDQKKLMSSNVQIVRQQSYSIMSIIKEEVLAYVVQLPIYGVIDTPCWKLHTSPLCTTNIKEGSNICLTRTDRGWYCDNAGSVSFFPQADRCKVQSNRVFCDTMYSLTLPSEVSLCNTDIFNSKYDCKIMTSKTDISSSVITSLGAIVSCYGKTKCTASNKNRGIIKTFSNGCDYVSNKGVDTVSVGNTLYYVNKLEGKNLYVKGEPIINYYDPLVFPSNEFYASISQVNEKINQSLAFIRRSDELLSAIGGYIPEAPRDGQAYVRKDGEWVLLSTFLGGSEPEA

SEQ ID NO: 10 - protein, 551 aa, Artificial Sequence
  RSV181180
MELLILKANAITTILTAVTFCFASGQNITEEFYQSTCSAVSRGYLSALRTGWYTSVITIELSNIKETKCNGTDTKVKLIKQELDKYKNAVTELQLLMQNTQAANNRARREAPQYMNYTINTTKNLNVSISKKRKRRFLGFLLGVGSAIASGMAVSKVLHLEGEVNKIKNALLSTNKAVVSLSNGVSVLTSKVLDLKNYINNQLLPIVNQQSCRIPNIETVIEFQQMNSRLLEITREFSVNAGVTTPLSTYMLTNSELLSLINDMPITNDQKKLMSSNVQIVRQQSYSIMSIIKEEVLAYVVQLPIYGVIDTPCWKLHTSPLCTTNIKEGSNICLTRTDRGWYCDNAGSVSFFPQADTCKVQSNRVFCDTMNSLTLPSEVSLCNTDIFNSKYDCKIMTSKTDISSSVITSLGAIVSCYGKTKCTASNKNRGIIKTFSNGCDYVSNKGVDTVSVGNTLYYVNKLEGKNLYVKGEPIINYYDPLVFPSNEFDASISQVNEKINQSLAFIRRSDELLSAIGGYIPEAPRDGQAYVRKDGEWVLLSTFLGGSEPEA

SEQ ID NO: 11 - protein, 551 aa, Artificial Sequence
  RSV181181
MELLILKANAITTILTAVTFCFASGQNITEEFYQSTCSAVSRGYLSALRTGWYTSVITIELSNIKETKCNGTDTKVKLIKQELDKYKNAVTELQLLMQNTQAANNRARREAPQYMNYTINTTKNLNVSISKKRKRRFLGFLLGVGSAIASGMAVSKVLHLEGEVNKIKNALLSTNKAVVSLSNGVSVLTSKVLDLKNYINNQLLPIVNQQSCRIPNIETVIEFQQMNSRLLEITREFSVNAGVTTPLSTYMLTNSELLSLINDMPITNDQKKLMSSNVQIVRQQSYSIMSIIKEEVLAYVVQLPIYGVIDTPCWKLHTSPLCTTNIKEGSNICLTRTDRGWYCDNAGSVSFFPQADTCKVQSNRVFCDTMNSLTLPSEVSLCNTDIFNSKYDCKIMTSKTDISSSVITSLGAIVSCYGKTKCTASNKNRGIIKTFSNGCDYVSNKGVDTVSVGNTLYYVNKLEGKNLYVKGEPIINYYDPLVFPSNEFYASISQVNEKINQSLAFIRRSDELLSAIGGYIPEAPRDGQAYVRKDGEWVLLSTFLGGSEPEA

SEQ ID NO: 12 - protein, 551 aa, Artificial Sequence
  RSV181182
MELLILKANAITTILTAVTFCFASGQNITEEFYQSTCSAVSRGYLSALRTGWYTSVITIELSNIKETKCNGTDTKVKLIKQELDKYKNAVTELQLLMQNTQAANNRARREAPQYMNYTINTTKNLNVSISKKRKRRFLGFLLGVGSAIASGMAVSKVLHLEGEVNKIKNALLSTNKAVVSLSNGVSVLTSKVLDLKNYINNQLLPIVNQQSCRIPNIETVIEFQQMNSRLLEITREFSVNAGVTTPLSTYMLTNSELLSLINDMPITNDQKKLMSSNVQIVRQQSYSIMSIIKEEVLAYVVQLPIYGVIDTPCWKLHTSPLCTTNIKEGSNICLTRTDRGWYCDNAGSVSFFPQADRCKVQSNRVFCDTMYSLTLPSEVSLCNTDIFNSKYDCKIMTSKTDISSSVITSLGAIVSCYGKTKCTASNKNRGIIKTFSNGCDYVSNKGVDTVSVGNTLYYVNKLEGKNLYVKGEPIINYYDPLVFPSNEFYASISQVNEKINQSLAFIRRSDELLSAIGGYIPEAPRDGQAYVRKDGEWVLLSTFLGGSEPEA

SEQ ID NO: 13 - protein, 544 aa, Artificial Sequence
  RSV180907
MELLIHRSSAIFLTLAINALYLTSSQNITEEFYQSTCSAVSRGYLSALRTGWYTSVITIELSNIKETKCNGTDTKVKLIKQELDKYKNAVTELQLLMQNTQAANNRARREAPQYMNYTINTTKNLNVSISKKRKRRFLGFLLGVGSAIASGMAVSKVLHLEGEVNKIKNALLSTNKAVVSLSNGVSVLTSKVLDLKNYINNQILPIVNQQSCRIPNIETVIEFQQMNSRLLEITREFSVNAGVTTPLSTYMLTNSELLSLINDMPITNDQKKLMSSNVQIVRQQSYSIMSIIKEEVLAYVVQLPIYGVIDTPCWKLHTSPLCTTNIKEGSNICLTRTDRGWYCDNAGSVSFFPQADTCKVQSNRVFCDTMNSLTLPSEVSLCNTDIFNSKYDCKIMTSKTDISSSVITSLGAIVSCYGKTKCTASNKNRGIIKTFSNGCDYVSNKGVDTVSVGNTLYYVNKLEGKNLYVKGEPIINYYDPLVFPSNEFDASISQVNEKINQSLAFIRRSDELLSAIGGYIPEAPRDGQAYVRKDGEWVLLSTFL

SEQ ID NO: 14 - protein, 544 aa, Artificial Sequence
  RSV180913
MELLILKANAITTILTAVTFCFASGQNITEEFYQSTCSAVSRGYLSALRTGWYTSVITIELSNIKETKCNGTDTKVKLIKQELDKYKNAVTELQLLMQNTQAANNRARREAPQYMNYTINTTKNLNVSISKKRKRRFLGFLLGVGSAIASGMAVSKVLHLEGEVNKIKNALLSTNKAVVSLSNGVSVLTSKVLDLKNYINNQILPIVNQQSCRIPNIETVIEFQQMNSRLLEITREFSVNAGVTTPLSTYMLTNSELLSLINDMPITNDQKKLMSSNVQIVRQQSYSIMSIIKEEVLAYVVQLPIYGVIDTPCWKLHTSPLCTTNIKEGSNICLTRTDRGWYCDNAGSVSFFPQADTCKVQSNRVFCDTMNSLTLPSEVSLCNTDIFNSKYDCKIMTSKTDISSSVITSLGAIVSCYGKTKCTASNKNRGIIKTFSNGCDYVSNKGVDTVSVGNTLYYVNKLEGKNLYVKGEPIINYYDPLVFPSNEFDASISQVNEKINQSLAFIRRSDELLSAIGGYIPEAPRDGQAYVRKDGEWVLLSTFL

SEQ ID NO: 15 - protein, 544 aa, Artificial Sequence
  RSV190417
MELLILKANAITTILTAVTFCFASGQNITEEFYQSTCSAVSRGYLSALRTGWYTSVITIELSNIKETKCNGTDTKVKLIKQELDKYKNAVTELQLLMQNTQAANNRARREAPQYMNYTINTTKNLNVSISKKRKRRFLGFLLGVGSAIASGMAVSKVLHLEGEVNKIKNALLSTNKAVVSLSNGVSVLTSKVLDLKNYINNQILPIVNQQSCRIPNIETVIEFQQMNSRLLEITREFSVNAGVTTPLSTYMLTNSELLSLINDMPITNDQKKLMSSNVQIVRQQSYSIMSIIKEEVLAYVVQLPIYGVIDTPCWKLHTSPLCTTNIKEGSNICLTRTDRGWYCDNAGSVSFFPQADTCKVQSNRVFCDTMNSLTLPSEVSLCNTDIFNSKYDCKIMTSKTDISSSVITSLGAIVSCYGKTKCTASNKNRGIIKTFSNGCDYVSNKGVDTVSVGNTLYYVNKLEGKNLYVKGEPIINYYDPLVFPSNEFYASISQVNEKINQSLAFIRRSDELLSAIGGYIPEAPRDGQAYVRKDGEWVLLSTFL

SEQ ID NO: 16 - protein, 544 aa, Artificial Sequence
  RSV190414
MELLILKANAITTILTAVTFCFASGQNITEEFYQSTCSAVSRGYLSALRTGWYTSVITIELSNIKETKCNGTDTKVKLIKQELDKYKNAVTELQLLMQNTQAANNRARREAPQYMNYTINTTKNLNVSISKKRKRRFLGFLLGVGSAIASGMAVSKVLHLEGEVNKIKNALLSTNKAVVSLSNGVSVLTSKVLDLKNYINNQILPIVNQQSCRIPNIETVIEFQQKNSRLLEITREFSVNAGVTTPLSTYMLTNSELLSLINDMPITNDQKKLMSSNVQIVRQQSYSIMSIIKEEVLAYVVQLPIYGVIDTPCWKLHTSPLCTTNIKEGSNICLTRTDRGWYCDNAGSVSFFPQADTCKVQSNRVFCDTMNSLTLPSEVSLCNTDIFNSKYDCKIMTSKTDISSSVITSLGAIVSCYGKTKCTASNKNRGIIKTFSNGCDYVSNKGVDTVSVGNTLYYVNKLEGKNLYVKGEPIINYYDPLVFPSNEFYASISQVNEKINQSLAFIRRSDELLSAIGGYIPEAPRDGQAYVRKDGEWVLLSTFL

SEQ ID NO: 17 - protein, 544 aa, Artificial Sequence
  RSV190420
MELLILKANAITTILTAVTFCFASGQNITEEFYQSTCSAVSRGYLSALRTGWYTSVITIELSNIKETKCNGTDTKVKLIKQELDKYKNAVTELQLLMQNTQAANNRARREAPQYMNYTINTTKNLNVSISKKRKRRFLGFLLGVGSAIASGMAVSKVLHLEGEVNKIKNALQLTNKAVVSLSNGVSVLTSKVLDLKNYINNQILPIVNQQSCRIPNIETVIEFQQKNSRLLEITREFSVNAGVTTPLSTYMLTNSELLSLINDMPITNDQKKLMSSNVQIVRQQSYSIMSIIKEEVLAYVVQLPIYGVIDTPCWKLHTSPLCTTNIKEGSNICLTRTDRGWYCDNAGSVSFFPQADTCKVQSNRVFCDTMNSLTLPSEVSLCNTDIFNSKYDCKIMTSKTDISSSVITSLGAIVSCYGKTKCTASNKNRGIIKTFSNGCDYVSNKGVDTVSVGNTLYYVNKLEGKNLYVKGEPIINYYDPLVFPSNEFYASISQVNEKINQSLAFIRRSDELLSAIGGYIPEAPRDGQAYVRKDGEWVLLSTFL

SEQ ID NO: 18 - protein, 544 aa, Artificial Sequence
  RSV200125
MELLILKANAITTILTAVTFCFASGQNITEEFYQSTCSAVSRGYLSALRTGWYTSVITIELSNIKETKCNGTDTKVKLIKQELDKYKNAVTELQLLMQNTQAANNRARREAPQYMNYTINTTKNLNVSISKKRKRRFLGFLLGVGSAIASGMAVSKVLHLEGEVNKIKNALQLTNKAVVSLSNGVSVLTSRVLDLKNYINNQILPMVNRQSCRIPNIETVIEFQQKNSRLLEITREFSVNAGVTTPLSTYMLTNSELLSLINDMPITNDQKKLMSSNVQIVRQQSYSIMSIIKEEVLAYVVQLPIYGVIDTPCWKLHTSPLCTTNIKEGSNICLTRTDRGWYCDNAGSVSFFPQADTCKVQSNRVFCDTMNSLTLPSEVSLCNTDIFNSKYDCKIMTSKTDISSSVITSLGAIVSCYGKTKCTASNKNRGIIKTFSNGCDYVSNKGVDTVSVGNTLYYVNKLEGKNLYVKGEPIINYYDPLVFPSNEFYASISQVNEKINQSLAFIRRSDELLSAIGGYIPEAPRDGQAYVRKDGEWVLLSTFL

SEQ ID NO: 19 - protein, 544 aa, Artificial Sequence
  RSV150042
MELLILKANAITTILTAVTFCFASGQNITEEFYQSTCSAVSKGYLSALRTGWYTSVITIELSNIKEIKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSIPNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSNEFDASISQVNEKINQSLAFIRKSDELLSAIGGYIPEAPRDGQAYVRKDGEWVLLSTFL

SEQ ID NO: 20 - protein, 503 aa, Artificial Sequence
  RSV150043
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELL

SEQ ID NO: 21 - protein, 460 aa, Artificial Sequence
  CR9506 heavy chain
EVQLVQSGAEVKKPGSSVKVSCKASGGTFSRSLITWVRQAPGQGLEWMGEISLVFGSAKNAQKFQGRVTITADESTSTAHMEMISLKHEDTAVYYCAAHQYGSGTHNNFWDESELRFDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - protein, 214 aa, Artificial Sequence
  CR9506 light chain
DIVMTQSPSSLSASVGDRVTIACRASQSIGTYLNWYQQKPGKAPKLLIYAASSLQSGVPSRFSGSGSGTHFTLAISSLQAEDFATYSCQQSYTIPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 23 - protein, 25 aa, Artificial Sequence
  RSV A fusion protein signal peptide
MELLILKANAITTILTAVTFCFASG

SEQ ID NO: 24 - protein, 25 aa, Artificial Sequence
  RSV B fusion protein signal peptide
MELLIHRSSAIFLTLAINALYLTSS

SEQ ID NO: 25 - protein, 574 aa, Artificial Sequence
  PR_wildtype
MELLILKANAITTILTAVTFCFASGQNITEEFYQSTCSAVSRGYLSALRTGWYTSVITIELSNIKETKCNGTDTKVKLIKQELDKYKNAVTELQLLMQNTPAANNRARREAPQYMNYTINTTKNLNVSISKKRKRRFLGFLLGVGSAIASGIAVSKVLHLEGEVNKIKNALQLTNKAVVSLSNGVSVLTSKVLDLKNYINNQLLPIVNQQSCRISNIETVIEFQQKNSRLLEITREFSVNAGVTTPLSTYMLTNSELLSLINDMPITNDQKKLMSSNVQIVRQQSYSIMSIIKEEVLAYVVQLPIYGVIDTPCWKLHTSPLCTTNIKEGSNICLTRTDRGWYCDNAGSVSFFPQADTCKVQSNRVFCDTMNSLTLPSEVSLCNTDIFNSKYDCKIMTSKTDISSSVITSLGAIVSCYGKTKCTASNKNRGIIKTFSNGCDYVSNKGVDTVSVGNTLYYVNKLEGKNLYVKGEPIINYYDPLVFPSDEFDASISQVNEKINQSLAFIRRSDELLHNVNTGKSTTNIMITAIIIVIIVVLLSLIAIGLLLYCKAKNTPVTLSKDQLSGINNIAFSN

SEQ ID NO: 26 - protein, 552 aa, Artificial Sequence
  SC_wildtype
MELLILKANAITTILTAVTFCFASGQNITEEFYQSTCSAVSRGYLSALRTGWYTSVITIELSNIKETKCNGTDTKVKLIKQELDKYKNAVTELQLLMQNTPAANNQARGSGSGRSLGFLLGVGSAIASGIAVSKVLHLEGEVNKIKNALQLTNKAVVSLSNGVSVLTSKVLDLKNYINNQLLPIVNQQSCRISNIETVIEFQQKNSRLLEITREFSVNAGVTTPLSTYMLTNSELLSLINDMPITNDQKKLMSSNVQIVRQQSYSIMSIIKEEVLAYVVQLPIYGVIDTPCWKLHTSPLCTTNIKEGSNICLTRTDRGWYCDNAGSVSFFPQADTCKVQSNRVFCDTMNSLTLPSEVSLCNTDIFNSKYDCKIMTSKTDISSSVITSLGAIVSCYGKTKCTASNKNRGIIKTFSNGCDYVSNKGVDTVSVGNTLYYVNKLEGKNLYVKGEPIINYYDPLVFPSDEFDASISQVNEKINQSLAFIRRSDELLHNVNTGKSTTNIMITAIIIVIIVVLLSLIAIGLLLYCKAKNTPVTLSKDQLSGINNIAFSN

SEQ ID NO: 27-28 - skipped (2 entries)

SEQ ID NO: 29 - protein, 574 aa, Artificial Sequence
  PR_stabilized
MELLILKANAITTILTAVTFCFASGQNITEEFYQSTCSAVSRGYLSALRTGWYTSVITIELSNIKETKCNGTDTKVKLIKQELDKYKNAVTELQLLMQNTQAANNRARREAPQYMNYTINTTKNLNVSISKKRKRRFLGFLLGVGSAIASGMAVSKVLHLEGEVNKIKNALQLTNKAVVSLSNGVSVLTSKVLDLKNYINNQLLPIVNQQSCRIPNIETVIEFQQKNSRLLEITREFSVNAGVTTPLSTYMLTNSELLSLINDMPITNDQKKLMSSNVQIVRQQSYSIMSIIKEEVLAYVVQLPIYGVIDTPCWKLHTSPLCTTNIKEGSNICLTRTDRGWYCDNAGSVSFFPQADTCKVQSNRVFCDTMNSLTLPSEVSLCNTDIFNSKYDCKIMTSKTDISSSVITSLGAIVSCYGKTKCTASNKNRGIIKTFSNGCDYVSNKGVDTVSVGNTLYYVNKLEGKNLYVKGEPIINYYDPLVFPSNEFYASISQVNEKINQSLAFIRRSDELLHNVNTGKSTTNIMITAIIIVIIVVLLSLIAIGLLLYCKAKNTPVTLSKDQLSGINNIAFSN

SEQ ID NO: 30 - protein, 552 aa, Artificial Sequence
  SC_stabilized
MELLILKANAITTILTAVTFCFASGQNITEEFYQSTCSAVSRGYLSALRTGWYTSVITIELSNIKETKCNGTDTKVKLIKQELDKYKNAVTELQLLMQNTQAANNQARGSGSGRSLGFLLGVGSAIASGMAVSKVLHLEGEVNKIKNALQLTNKAVVSLSNGVSVLTSKVLDLKNYINNQILPIVNQQSCRIPNIETVIEFQQKNSRLLEITREFSVNAGVTTPLSTYMLTNSELLSLINDMPITNDQKKLMSSNVQIVRQQSYSIMSIIKEEVLAYVVQLPIYGVIDTPCWKLHTSPLCTTNIKEGSNICLTRTDRGWYCDNAGSVSFFPQADTCKVQSNRVFCDTMNSLTLPSEVSLCNTDIFNSKYDCKIMTSKTDISSSVITSLGAIVSCYGKTKCTASNKNRGIIKTFSNGCDYVSNKGVDTVSVGNTLYYVNKLEGKNLYVKGEPIINYYDPLVFPSNEFDASISQVNEKINQSLAFIRRSDELLHNVNTGKSTTNIMITAIIIVIIVVLLSLIAIGLLLYCKAKNTPVTLSKDQLSGINNIAFSN

SEQ ID NO: 31 - DNA, 1722 bp, Artificial Sequence
  Ad26RSV019
atggaactgctgatcctgaaggccaacgccatcaccacaatcctgaccgccgtgaccttttgcttcgccagcggccagaacatcaccgaggaattctaccagagcacctgtagcgccgtgtccagaggatatctgtctgccctgagaaccggctggtacaccagcgtgatcaccatcgagctgagcaacatcaaagaaacaaagtgcaacggcaccgacaccaaagtgaagctgatcaagcaagagctggacaagtacaagaatgccgtgaccgaactgcagctgctgatgcagaatacccaggccgccaacaaccgggccagaagagaagcccctcagtacatgaactacaccatcaacaccaccaagaacctgaacgtgtccatcagcaagaagcggaagcggagattcctgggctttctgctcggagtgggatctgccattgcctctggaatggccgtgtctaaggtgctgcatctggaaggcgaagtgaacaagatcaagaacgccctgcagctgaccaacaaggccgtggtgtctctgtctaatggcgtgtccgtgctgaccagcagagtgctggacctgaagaactacatcaacaaccagctgctgcccatggtcaaccggcagagctgcagaatccccaacatcgagacagtgatcgagttccagcagaagaacagcaggctgctggaaatcacccgcgagttttctgtgaatgccggcgtgacaacccctctgagcacctacatgctgaccaatagcgagctgctgagcctgatcaacgacatgcccatcaccaacgaccagaaaaagctgatgagcagcaacgtgcagatcgtgcggcagcagagctacagcatcatgagcattatcaaagaagaggtgctggcctacgtggtgcagctgcctatctacggcgtgatcgataccccttgctggaagctgcacacaagcccactgtgcaccaccaatatcaaagagggcagcaacatctgcctgaccagaaccgatagaggctggtactgcgataatgccggcagcgtcagcttcttcccacaagccgatacctgcaaggtgcagagcaacagagtgttctgcgacaccatgaacagcctgacactgcctagcgaggtgtccctgtgcaacaccgacatcttcaactctaagtacgactgcaagatcatgacctccaagaccgacatcagctcctccgtgatcacatctctgggcgccatcgtgtcctgctacggcaagacaaagtgtaccgccagcaacaagaaccggggcatcatcaagaccttcagcaacggctgcgactacgtgtccaacaaaggcgtggacaccgtgtctgtgggcaacaccctgtactacgtgaacaagctggaaggcaagaatctgtacgtgaagggcgagcccatcatcaactactacgaccctctggtgttccccagcaacgagttctacgccagcatcagccaagtgaacgagaagatcaaccagagcctggccttcatccgcagatccgatgagctgctgcacaacgtgaacaccggcaagagcaccacaaacatcatgatcaccgccatcatcatcgtgatcatcgtcgtgctgctgtccctgatcgccatcggactgctgctgtactgcaaggccaagaacacccctgtgacactgagcaaggatcagctgagcggcatcaacaatatcgccttctccaac

SEQ ID NO: 32 - protein, 574 aa, Artificial Sequence
  The amino acid sequence of the transgene (RSV-B preF protein, processed)
MELLILKANAITTILTAVTFCFASGQNITEEFYQSTCSAVSRGYLSALRTGWYTSVITIELSNIKETKCNGTDTKVKLIKQELDKYKNAVTELQLLMQNTQAANNRARREAPQYMNYTINTTKNLNVSISKKRKRRFLGFLLGVGSAIASGMAVSKVLHLEGEVNKIKNALQLTNKAVVSLSNGVSVLTSRVLDLKNYINNQLLPMVNRQSCRIPNIETVIEFQQKNSRLLEITREFSVNAGVTTPLSTYMLTNSELLSLINDMPITNDQKKLMSSNVQIVRQQSYSIMSIIKEEVLAYVVQLPIYGVIDTPCWKLHTSPLCTTNIKEGSNICLTRTDRGWYCDNAGSVSFFPQADTCKVQSNRVFCDTMNSLTLPSEVSLCNTDIFNSKYDCKIMTSKTDISSSVITSLGAIVSCYGKTKCTASNKNRGIIKTFSNGCDYVSNKGVDTVSVGNTLYYVNKLEGKNLYVKGEPIINYYDPLVFPSNEFYASISQVNEKINQSLAFIRRSDELLHNVNTGKSTTNIMITAIIIVIIVVLLSLIAIGLLLYCKAKNTPVTLSKDQLSGINNIAFSN

SEQ ID NO: 33 - DNA, 1656 bp, Artificial Sequence
  Ad26RSV020
atggaactgctgatcctgaaggccaacgccatcaccacaatcctgaccgccgtgaccttttgcttcgccagcggccagaacatcaccgaggaattctaccagagcacctgtagcgccgtgtccagaggatatctgtctgccctgagaaccggctggtacaccagcgtgatcaccatcgagctgagcaacatcaaagaaacaaagtgcaacggcaccgacaccaaagtgaagctgatcaagcaagagctggacaagtacaagaatgccgtgaccgaactgcagctgctgatgcagaatacccaggccgccaacaatcaggccagaggctctggatctggcagaagcctgggatttctgctcggcgtgggatctgccattgcctctggaatggccgtgtctaaggtgctgcatctggaaggcgaagtgaacaagatcaagaacgccctgcagctgaccaacaaggccgtggtgtctctgtctaatggcgtgtccgtgctgaccagcagagtgctggacctgaagaactacatcaacaaccagctgctgcccatggtcaaccggcagagctgcagaatccccaacatcgagacagtgatcgagttccagcagaagaacagcaggctgctggaaatcacccgcgagttttctgtgaatgccggcgtgacaacccctctgagcacctacatgctgaccaatagcgagctgctgagcctgatcaacgacatgcccatcaccaacgaccagaaaaagctgatgagcagcaacgtgcagatcgtgcggcagcagagctacagcatcatgagcattatcaaagaagaggtgctggcctacgtggtgcagctgcctatctacggcgtgatcgataccccttgctggaagctgcacacaagcccactgtgcaccaccaatatcaaagagggcagcaacatctgcctgaccagaaccgatagaggctggtactgcgataatgccggcagcgtcagcttcttcccacaagccgatacctgcaaggtgcagagcaacagagtgttctgcgacaccatgaacagcctgacactgcctagcgaggtgtccctgtgcaacaccgacatcttcaactctaagtacgactgcaagatcatgacctccaagaccgacatcagctcctccgtgatcacatctctgggcgccatcgtgtcctgctacggcaagacaaagtgtaccgccagcaacaagaaccggggcatcatcaagaccttcagcaacggctgcgactacgtgtccaacaaaggcgtggacaccgtgtctgtgggcaacaccctgtactacgtgaacaagctggaaggcaagaacctgtacgtgaagggcgagcccatcatcaactactacgaccctctggtgttccccagcaacgagttcgatgccagcatcagccaagtgaacgagaagatcaaccagagcctggccttcatcagacgctccgatgagctgctgcacaacgtgaacaccggcaagagcaccacaaacatcatgatcaccgccatcatcatcgtgatcatcgtcgtgctgctgtccctgatcgccatcggactgctgctgtactgcaaggccaagaacacccctgtgacactgagcaaggatcagctgagcggcatcaacaatatcgccttctccaac

SEQ ID NO: 34 - protein, 552 aa, Artificial Sequence
  protein encoded by Ad26RSV020 (single chain)
MELLILKANAITTILTAVTFCFASGQNITEEFYQSTCSAVSRGYLSALRTGWYTSVITIELSNIKETKCNGTDTKVKLIKQELDKYKNAVTELQLLMQNTQAANNQARGSGSGRSLGFLLGVGSAIASGMAVSKVLHLEGEVNKIKNALQLTNKAVVSLSNGVSVLTSRVLDLKNYINNQLLPMVNRQSCRIPNIETVIEFQQKNSRLLEITREFSVNAGVTTPLSTYMLTNSELLSLINDMPITNDQKKLMSSNVQIVRQQSYSIMSIIKEEVLAYVVQLPIYGVIDTPCWKLHTSPLCTTNIKEGSNICLTRTDRGWYCDNAGSVSFFPQADTCKVQSNRVFCDTMNSLTLPSEVSLCNTDIFNSKYDCKIMTSKTDISSSVITSLGAIVSCYGKTKCTASNKNRGIIKTFSNGCDYVSNKGVDTVSVGNTLYYVNKLEGKNLYVKGEPIINYYDPLVFPSNEFDASISQVNEKINQSLAFIRRSDELLHNVNTGKSTTNIMITAIIIVIIVVLLSLIAIGLLLYCKAKNTPVTLSKDQLSGINNIAFSN